(19) United States
(12) Patent Application Publication
(10) Pub. No.: US 2026/0049319 A1
(43) Pub. Date: Feb. 19, 2026

(54) TREM2 BINDS TO SPECIFICALLY STRUCTURED HEPARAN SULFATE

(71) Applicant: University of South Florida, Tampa, FL (US)

(72) Inventors: Lianchun Wang, Tampa, FL (US); IIayda Ozsan McMillan, Tampa, FL (US)

(21) Appl. No.: 19/302,783

(22) Filed: Aug. 18, 2025

Related U.S. Application Data
(60) Provisional application No. 63/684,118, filed on Aug. 16, 2024.
(60) Provisional application No. 63/764,898, filed on Feb. 28, 2025.

(51) Int. Cl. C12N 15/113; A61K 39/00; A61P 25/28; C07K 16/28

(52) U.S. Cl. CPC C12N 15/1137; A61P 25/28; C07K 16/2803; A61K 2039/505; C07K 2317/75; C07K 2317/92; C12N 2310/14

(57) ABSTRACT

The present disclosure relates to methods of treating and/or preventing neurodegenerative diseases by modulating interactions between the heparan sulfate-Triggering Receptor Expressed on Myeloid Cell-2 (HS-TREM2) complex and preventing accumulation of amyloid-β (Aβ).

### RELATED APPLICATION

This U.S. utility application claims priority to, and the benefit of, U.S. Provisional Patent Application No. 63/684,118, filed Aug. 16, 2024, entitled “TREMS2 BINDS TO SPECIFICALLY STRUCTURED HEPARAN SULFATE”, and U.S. Provisional Patent Application No. 63/764,898, filed Feb. 28, 2025, entitled “TREM2 BINDS TO SPECIFICALLY STRUCTURED HEPARAN SULFATE,” which are each incorporated by reference herein in their entirety.

### REFERENCE TO SEQUENCE LISTING

The sequence listing submitted on Aug. 18, 2025, as an .XML file entitled “11001-229US1_ST26” created on Aug. 1, 2025, and having a file size of 23,905 bytes is hereby incorporated by reference pursuant to 37 C.F.R. § 1.52 (e) (5).

### FIELD

The present disclosure relates methods of treating and/or preventing neurodegenerative diseases by preventing accumulation of amyloid-β (AB).

### BACKGROUND

Numerous progressive neurodegenerative disorders, such as Alzheimer's disease (AD), are characterized by amyloid-β (Aβ) deposition, intraneuronal neurofibrillary tangles, and widespread neuroinflammation. Currently, no effective treatments exist to halt or significantly slow neurodegenerative disease progression. With focus on AD, uncovering novel mechanisms underlying AD pathogenesis is crucial for developing efficacious therapeutic interventions. Mounting evidence indicates that Aβ accumulation in the brain is a pivotal early event in AD pathology, with kinetic studies indicating that faulty Aβ clearance, rather than overproduction, is central to Aβ accumulation in sporadic AD patients. Currently, the molecular mechanisms driving Aβ clearance remain poorly understood, and what is need in the field are therapeutic agents that can drive Aβ clearance to treat and/or prevent progression of neurodegenerative diseases, such as AD.

The methods disclosed herein address the need for said therapeutic agents that can reduce accumulation of Aβ.

### SUMMARY

The present disclosure provides methods of treating and/or preventing a neurodegenerative disease by administering inhibitors of heparan sulfate (HS) biosynthetic enzymes or inhibitors of HS-Triggering Receptor Expressed on Myeloid Cell-2 (HS-TREM2) complex formation. The present disclosure provides methods of treating and/or preventing a neurodegenerative disease by administering an agonist/activator of TREM2 activity. The present disclosure also provides methods of reducing accumulation of amyloid-β (Aβ).

In one aspect, disclosed herein is a method of treating a neurodegenerative disease, the method comprising administering to a subject in need thereof a pharmaceutical composition comprising at least one inhibitor of a heparan sulfate (HS) biosynthetic enzyme, wherein the at least one inhibitor decreases formation of a HS-Triggering Receptor Expressed on Myeloid Cells-2 (HS-TREM2) complex and decreases accumulation of Aβ relative to an untreated control.

In some embodiments, the at least one inhibitor of a HS biosynthetic enzyme comprises a peptide, an oligonucleotide, a small molecule, or an immunoglobulin. In some embodiments, the at least one inhibitor of a HS biosynthetic enzyme comprises an inhibitor of N-deacetylase/N-sulfotransferases-1 (NDST1), an inhibitor of a Exostosin (EXT) glycosyltransferase, an inhibitor of a HS 6-O-sulfotransferase (HS6ST), an inhibitor of a glucuronyl C5-epimerase (Glce), or a combination thereof.

In some embodiments, the inhibitor of NDST1 inhibits binding between HS and TREM2. In some embodiments, the inhibitor of the EXT glycosyltransferase inhibits adding a sugar molecule to a heparan sulfate chain. In some embodiments, the EXT glycosyltransferase comprises an EXT1 or an EXT2. In some embodiments, the HS6ST comprises HS6ST1, HS6ST2, HS6ST3, or any variant thereof. In some embodiments, the inhibitor of HS6ST or the inhibitor of Glce inhibits a modification to HS.

In some embodiments, the modification comprises sulfonation to a glucosamine residue of HS. In some embodiments, the modification comprises converting glucuronic acid (GlcA) to iduronic acid (IdoA).

In some embodiments, the neurodegenerative disease comprises Alzheimer's Disease (AD). In some embodiments, the method promotes phagocytosis and degradation of Aβ.

In some aspects, disclosed herein is a method thereof a pharmaceutical composition comprising at least one agonist of a Triggering Receptor Expressed on Myeloid Cells-2 (TREM2), wherein the at least one agonist of TREM2 increases binding of amyloid-β (Aβ) to the TREM2 and decreases accumulation of Aβ relative to an untreated control.

In some embodiments, the at least one agonist of TREM2 comprises a peptide, an oligonucleotide, a small molecule, or an immunoglobulin. In some embodiments, the neurodegenerative disease comprises Alzheimer's Disease (AD). In some embodiments, the method promotes phagocytosis and degradation of Aβ.

In some aspects, disclosed herein is a method of reducing accumulation of Aβ, the method comprising contacting the cell with at least one inhibitor of a heparan sulfate (HS) biosynthetic enzyme, at least one agonist of a Triggering Receptor Expressed on Myeloid Cells-2 (TREM2), or a combination thereof, wherein the method decreases formation of a HS-TREM2 complex and increases binding of Aβ to the TREM2.

In some embodiments, the at least one inhibitor of a HS biosynthetic enzyme comprises a peptide, an oligonucleotide, a small molecule, or an immunoglobulin. In some embodiments, the at least one agonist of TREM2 comprises a peptide, an oligonucleotide, a small molecule, or an immunoglobulin. In some embodiments, the method treats or prevents Alzheimer's disease.

### BRIEF DESCRIPTION OF FIGURES

The accompanying figures, which are incorporated in and constitute a part of this specification, illustrate several aspects described below.

FIGS. 1A, 1B, 1C, 1D, 1E, and IF show that TREM2 interacts with HS, forming binary complexes on the microglia cell surface. FIG. 1A shows the concentration-dependent binding of TREM2 to wildtype Ndst1^{f/f} MLEC surface. A background value of 0.281 (no TREM2 added) was subtracted. FIG. 1B shows the binding of TREM2 to wildtype Ndst1^{f/f} MLEC surface in the presence of heparin. FIG. 1C shows the binding of TREM2 to Ext1^{-/-} mutant MLEC surface. Ext1^{-/-} MLECs are isogenic mutants derived from Ext1^{f/f} cells after the conditional deletion of Ext1 using Adeno-Cre. FIG. 1D shows the binding of TREM2 to wild-type Ndst1^{f/f} MLECs after treatment with heparinases I and III or chondroitinase ABC (Chon'ase ABC). The background without streptavidin-HRP addition background is also included. FIG. 1E shows the immunofluorescence staining of TREM2 and HS (using the 10E4 antibody) expressed on the C20 cell surface, with co-localization assessed by Pearson's R correlation analysis. Scale bars represent 25 μm. FIG. 1F shows the proximity ligation assay demonstrating HS-TREM2 binary complexes on the microglial C20 cell surface. The TREM2-HS puncta numbers were calculated by normalizing to nuclear DAPI staining. Data are presented as mean±SEM and are representative of three independent experiments with statistical significance determined by an unpaired t-test. Scale bars represent 25 μm.

FIGS. 2A, 2B, 2C, 2D, and 2E show the interaction of TREM2 with heparins of different sizes and chemical modifications. FIG. 2A shows the depiction of the structures of chemically modified heparins. FIG. 2B shows the SPR binding sensorgram demonstrates strong TREM2 binding affinity to immobilized heparin, with a KD calculated at 1.37±1.28×10\hat{ }−9 M. FIGS. 2C, 2D, and 2E show the competitive SPR assays involved preincubating TREM2 (250 nM) with 1000 nM heparin oligomers of varying sizes (FIG. 2C) or with 1000 nM heparin, N-Desulfated Heparin (N-Des Hep), 2-Desulfated Heparin (2-Des Hep), or 6-Desulfated Heparin (6-Des Hep) (FIGS. 2D and 2E). These results are representative of three independent experiments.

FIGS. 3A and 3B show that the low molecular weight (LMW) HS microarray reveals TREM2 binds to HS structures containing 6-O-sulfates and Iduronic acid modifications. FIG. 3A shows the fluorescence intensity of bound TREM2 to wells immobilized with chemoenzymatically synthesized low molecular weight HS with different structures. TREM2 with his-tag (10 μg/ml) was screened against 96 LMW HS compounds printed at 25 μM, and bound TREM2 was visualized and quantified using AF488 NTA Ni. Each compound was printed 12 times, resulting in 12 spots per compound. The binding threshold for this experiment was set at 1000 fluorescence intensity (U), depicted by a black dashed line. Results are represented as mean±SD for each compound. FIG. 2B shows the comparison of TREM2 binding HS oligosaccharides (HS #74, 76, 78, 84, and 96) and compounds that do not bind TREM2 (HS #23, 24, 47, 48, 68, 73, 75 and 77).

FIGS. 4A, 4B, 4C, 4D, 4E, 4F, and 4G show the binding of TREM2 to HS mutant MLEC cell surfaces. FIG. 4A shows the generated HS mutant MLEC lines, which express differently structured HS. FIG. 4B shows the comparison between Hs6st1^{-/-} vs. corresponding isogenic wildtype Hs6st1^{f/f} MLECs. FIG. 4C shows the comparison between Hs6st1^{-/-}; 2^{-/-} vs corresponding isogenic wildtype Hs6st1^{f/f} MLECs. FIG. 4D shows the comparison between Hs2st^{-/-} vs corresponding isogenic wildtype Hs2st^{f/f} MLECs. FIG. 4E shows the comparison between Ndst1^{-/-} vs. corresponding isogenic wildtype Ndst1^{f/f} MLECs. FIG. 4F shows the comparison between Glce^{-/-} vs. corresponding isogenic wildtype Ndst1^{f/f} MLECs. FIG. 4G shows the comparison between Hs3st1^{-/-} vs corresponding isogenic wildtype Ndst1^{f/f} MLECs. The data presented are representative of three independent experiments and are expressed as mean±SEM. An unpaired t-test was applied for statistical analysis.

FIGS. 5A, 5B, 5C, 5D, 5E, and 5F show that the Ext1 and Hs6sts are required for TREM2-HS binary complex formation on the microglial cell surface. FIG. 5A shows the expression profiles of Ext1, Ext2, and Hs6st1-3 in C20 microglia cells assessed by quantitative RT-PCR analysis. FIG. 5B shows the siRNA knockdown (KD) of TREM2, Ext1, Hs6st1, and Hs6st3 in C20 cells compared to empty vector-transfected cells (normal control, NC). FIGS. 5C and 5D show the impact of Ext1, Hs6st1, or Hs6st3 knockdown on the colocalization of TREM2 and HS on C20 cell surface. FIGS. 5E and 5F show the effect of Ext1, Hs6st1, or Hs6st3 knockdown on TREM2-HS binary complex formation on C20 cell surface. Data are presented as mean±SEM and are representative of three independent experiments. Scale bars represent 25 μm.

FIGS. 6A, 6B, 6C, and 6D show the impact of TREM2, Ext1, Hs6st1, and Hs3st6 on ApoE uptake by C20 microglia. FIGS. 6A and 6B show the TREM2 knockdown in C20 cells results in decreased uptake of ApoE-488 compared to cells transfected with an empty vector.

FIGS. 6C and 6D show the knockdown of Ext1, Hs6st1, or Hs3st3 in C20 cells leads to reduced uptake of ApoE-488 compared to cells transfected with an empty vector. Scale bars represent 100 μm. Data are presented as mean±SEM and are representative of three independent experiments.

FIG. 7 shows TREM2 binging to wildtype Ext1^{f/f} MLECs in the presence of heparin (100 μg/mL).

FIG. 8 shows the compound numbering (Cmpd #) and structures with symbol nomenclature for low molecular weight HS structures on the microarray. Cmpds #1-96 are modified at the reducing end to contain an amine linker for adherence to the microarray chip.

FIGS. 9A and 9B show the low molecular weight (LMW) HS microarray of antithrombin III binding. FIG. 9A shows the raw fluorescent results of the antithrombin III binding LMW HS microarray and corresponding HS code (numbered 1-96). FIG. 9B shows the complete results of antithrombin binding to an LMW HS microarray were visualized with OG488, and the 5 HS oligosaccharides known to bind antithrombin all showed positive binding in the assay.

FIG. 10 shows the raw fluorescent results of TREM2 binding LMW HS microarray and corresponding HS code.

FIG. 11 shows that the ligand structure prior to docking as depicted as sticks, with colors corresponding to the atomic chemical elements (upper figure). The structure of heparin octasaccharide used for docking (lower figure). Top docked pose for the heparin fragment with TREM2. The protein structure is shown as a solvent accessible surface colored according to the electrostatic potential from red to blue indicating electrostatically negative to positive.

FIGS. 12A and 12B show the central conception of the present disclosure. FIG. 12A shows that in the presence of Aβ, microglial surface TREM2 binds Aβ to mediate Aβ clearance (1) and upregulate TREM2 expression (2). These processes are inhibited by HS, which forms HS-TREM2 complexes that block TREM2-Aβ interaction. Additionally, HS independently suppresses TREM2 expression (3). FIG. 12B shows that reducing microglial HS or its 6-O-sulfation (not shown) alleviates these inhibitory effects, enhancing TREM2-mediated Aβ clearance.

FIGS. 13A, 13B, 13C, 13D, 13E, 13F, and 13G show reduced microglial HS expression in AD patients and 5×FAD mice. FIG. 13A shows that HS is modified by NS, 2S, 3S and 6S, which form ligand-binding sites. HS synthesis begins with polymerization of N-acetylglucosamine (GlcNAc) and glucuronic acid (GlcA) residues by the Exotosin-1 (Ext1)-Ext2 heterodimer co-polymerase, followed by modifications: NS by N-deacetylase/N-sulfotransferases (Ndst1-4), GlcA epimerization to iduronic acid (IdoA) by Epimerase (Glce), 2S by HS 2-O-sulfotransferase 1 (Hs2st1), 6S by 6-O-sulfotransferases (Hs6st1-3), and 3S by 3-O-Sulfotransferases (Hs3st1-6). Gal, galactose; Xyl, xylose. FIG. 13B shows that scRNA-seq shows downregulation of Ext1 in microglia from human AD forebrains. Ndst1 reduction was observed but not statistically tested (NA). Dataset: GEO: syn18485175; n=24 per group for control and AD. FIGS. 13C and 13D show the co-immunostaining of Iba1 and HS (10E4 antibody) reveals reduced HS in microglia in AD forebrains, especially in DAM (within 15 μm from Aβ plaques circled by white dashed lines). Yellow triangles indicate microglia expressing high (filled) or low (empty) HS. Images represent 4 AD (3 females, 1 male, Braak V-VI) and 4 age-matched female controls. T-test was used for comparison. Note: HS co-deposits with Aβ and is also highly expressed in capillary (denoted by empty white arrows). HS in other cells is much lower and could not be seen at our chosen image acquisition time. FIG. 13E shows the proportions of HM, DAM1, and DAM2 in 6-month-old 5×FAD mice. HM includes all non-DAM microglia. Our own scRNA-seq study used the updated marker genes reported in the most recent literature to analyze the microglia subpopulations in our microglia-specific Ndst1 knockout mice without or with a 5×FAD heterozygous background (see FIG. 15). FIG. 13F shows that scRNA-seq shows progressive downregulation of Ndst1 and Hs6st1 as HM transitions to DAM1 and DAM2 in 6-month-old 5×FAD mice. 13G shows the bulk RNA-seq of primary mouse microglia treated with Aβ fibrils (fAβ, 1 or 12 hours) and oligomers (oAβ, 12 hours) shows overall HS gene downregulation (summarized from public scRNA-seq dataset from publication: □PMCID: PMC8321667; DOI: 10.26508/lsa.202101108). Despite Ext1 upregulation, reduced Ext2 and HS modification genes, especially Ndst1, suggest an overall reduced HS expression. *, p<0.05.

FIGS. 14A, 14B, 14C, 14D, 14E, 14F, 14G, and 14H show that knockout of microglial Ndst1 reduces Aβ deposition, enhances Aβ phagocytosis and improves cognition in 5×FAD mice. FIG. 14A shows the immunostaining of primary Ndst1^{f/f} and Ndst1^{CKO} microglia using anti-HS (10E4) and anti-Iba1 antibodies show reduced HS in Ndst1^{CKO} microglia. FIG. 14B shows the timeline of Aβ pathology in 5×FAD mice. FIG. 14C shows the anti-human Aβ (D52D2) staining shows reduced Aβ deposition in 6-month-old Ndst1^{CKO}; 5×FAD mice vs. 5×FAD mice. No Aβ was detected in Ndst1^{f/f} and Ndst1^{CKO} mice (data not shown). N=3-5 mice/group. FIG. 14D shows the in vivo Methoxy-X04 phagocytosis assay show higher fluorescence in Ndst1^{CKO}; 5×FAD microglia vs. 5×FAD microglia, measured by flow cytometry 3 hours post i.p. injection. N=4 mice/group. FIG. 14E shows the Aβ-HL88 fibril uptake was higher in heparinase-treated 5×FAD and Ndst1^{CKO}; 5×FAD microglia vs. 5×FAD microglia after 18 hours of incubation. FIG. 14F NOR test. Ndst1^{CKO}; 5×FAD mice showed a higher discrimination index than 5×FAD mice. FIGS. 14G and 14H show the Y-maze test. The Ndst1^{CKO}; 5×FAD mice had a higher alternation and fewer entries than 5×FAD controls. N=5-7 male mice/group. Data are mean±SEM, with statistical analysis by Student's t-test.

FIGS. 15A, 15B, 15C, 15D, and 15E show the microglia scRNA-seq analysis. FIG. 15A shows the UMAP plots displaying HM, TM1, TM2 and DAM in 5×FAD and Ndst1^{CKO}; 5×FAD mice (2 mice/genotype). Sub-cluster annotation was based on recently reported signature genes. FIG. 15B shows the average scaled expression levels of selected signature genes for HM, TM1, TM2, and DAM. FIGS. 15C and 15D show the HM, TM1, TM2 and DAM proportion in 5×FAD and Ndst1^{CKO}; 5×FAD microglia (FIG. 15C) and their transition efficiency, an index calculated by normalizing the proportional contribution of the subsequent development stage to the current stage (FIG. 15D). FIG. 15E shows the gene set enrichment analysis of differentially expressed genes in HM, TM1, TM2 and DAM clusters between Ndst1^{CKO}; 5×FAD and 5×FAD microglia.

FIGS. 16A, 16B, 16C, and 16D show the Ndst1 deletion upregulates TREM2 expression in microglia. FIGS. 16A and 16B show that the scRNA-seq revealed that TREM2 expression is upregulated in Ndst1^{CKO} microglia with or without 5×FAD (FIG. 16A). Without 5×FAD, TREM2 upregulation is only significant in DAM, and with 5×FAD, TREM2 was upregulated across HM, TM1, TM2 and DAM populations (FIG. 16B). FIG. 16C shows that the qRT-PCR analysis detected higher TREM2 expression in Ndst1^{CKO} mouse brains compared to Ndst1^{f/f} brains. n=4-5 mice/genotype. FIG. 16D shows the Ndst1^{CKO} primary microglia showed a higher TREM2 antibody staining than Ndst1^{f/f} microglia. Data are presented as Mean±SEM, with t-test for statistical analysis.

FIGS. 17A, 17B, 17C, and 17D show the formation of HS-TREM2 binary complexes on the human microglia cell surface. FIGS. 17A and 17B shows the immunostaining of C20 cell surface TREM2 and HS, with co-localization assessed by Pearson's R correlation analysis. FIGS. 17C and 17D show the PLA demonstrating HS-TREM2 binary complexes on the microglial C20 cell surface, and the complex formation was diminished on the cells pretreated with heparinases I-III or Ext1 knockdown (KD). The TREM2-HS puncta numbers were normalized to nuclear DAPI staining. Data are mean±SEM and representative of three independent experiments with statistical significance determined by unpaired t-test.

FIGS. 18A, 18B, and 18C show that the Ndst1^{CKO} BMDMs show diminished HS-TREM2 complex formation and heparin binding blocks TREM2-Aβ interaction. FIG. 18A shows the PLA of HS-TREM2 complexes on the surface of Ndst1^{f/f} and Ndst1^{KO} BMDMs. FIG. 18B shows the TREM2-Heparin/Aβ ELISA. Enoxaparin (0-12.5 μg/ml) was incubated with TREM2-coated plate (2 μg/ml), followed by Aβ42 fibrils (1 μg/ml). Bound Aβ42 was quantified using HRP-conjugated anti-Aβ42 antibody. FIG. 18C shows the TREM2-Heparin/Aβ pulldown assay. Biotinylated-TREM2 (2 μg) was preincubated with Enoxaparin (200 μg/ml) or heparin (2 and 200 g/ml), then incubation with Aβ42 fibrils (1.5 μg). Streptavidin-beads pulled-downs were analyzed by Western Blot. Data are mean±SEM with statistical significance determined by unpaired t-test.

FIGS. 19A and 19B show the knockdown (KD) of Ext1, Ndst1, or Hs6st1 in C20 microglia increased migration toward Aβ in a transwell migration assay. Aβ was preincubated in serum-free medium for 8 hours before use. TREM2 KD abolished the enhanced migration of C20 cells with KD of Ext1, Ndst1, or Hs6st1. Data are shown after subtracting the PBS control. Primary Ndst1 (KO microglia exhibited higher Green DND-26 fluorescence, indicating increased lysosomal activity, compared to Ndst1^{f/f} controls. Data presented as mean±SEM. Significance determined by t-test.

FIGS. 20A, 20B, 20C, 20D, and 20E show that Hs6st1 deficiency diminishes HS-TREM2 complexes and enhances Aβ uptake in microglia. FIGS. 20A and 20B shows that PLA shows diminished HS-TREM2 complexes (green dots) on the cell surface of Hs6st1 KD (Hs6st1^{KD}) C20 microglia. FIG. 20C shows that the knockdown of Ndst1, Ext1 or Hs6st1 increases Aβ fibrils uptake in C20 microglia, while TREM2 KI) reduces the increased Aβ fibrils uptake in the double KD (dKD) cells. FIGS. 20D and 20E show that PLA demonstrates diminished HS-TREM2 complexes (red dots) on the cell surface of Hs6st1^{KO} and heparinases-pretreated BMDMs. Data are representative of 2-3 separate experiments and presented as mean±SEM. Significance determined by unpaired t-test.

FIG. 21 shows the Microglial Ext1, Ndst1, and Hs6st1 expression in control (n=24), early (n=15), and later (n=9) stage AD patients. Dataset: GEO: syn18485175.

FIGS. 22A and 22B show schematics and donor/recipient profiles. FIG. 22A shows the schematic of the BMDM replacement initiated at Aβ deposition onset. FIG. 22B shows the donor and recipient profiles.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction below, above, or in between the given ranges as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treat,” “treating,” and grammatical variations thereof as used herein, include partially or completely delaying, alleviating, mitigating or reducing the intensity of one or more attendant symptoms of a disease or disorder, including but not limited to AD, and/or alleviating, mitigating or impeding one or more causes of a disease or disorder, including but not limited to AD, frontotemporal dementia, Parkinson's Disease, Nasu-Hakola diseases, a cancer, and atherosclerosis.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a disease, including but not limited to AD, frontotemporal dementia, Parkinson's Disease, Nasu-Hakola diseases, a cancer, and atherosclerosis). The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

The term “variant” means a polypeptide derived from a parent polypeptide by one or more (several) alteration(s), i.e., a substitution, insertion, and/or deletion, at one or more (several) positions. A substitution means a replacement of an amino acid occupying a position with a different amino acid, a deletion means removal of an amino acid occupying a position: and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X,’ the insertion may be at position ‘X+1’ (‘downstream’) or at position ‘X−1’ (‘upstream’).

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide. A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

“Inhibitors” or “antagonist” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is about 80%, optionally 50% or 25, 10%, 5%, or 1% or less.

A “variant” or a “derivative” of a particular inhibitor may be defined as a chemical or molecular compound having at least 50% identity to a parent or original inhibitor. In some embodiments a variant inhibitor may show, for example, at least 60%, at least 70%, at least 80%, at least 90%, at least 91%, at least 92%, at least 93%, at least 94%, at least 95%, at least 96%, at least 97%, at least 98%, or at least 99% or greater identity relative to a reference parent or original inhibitor.

As used herein, the term “agonist” or “activator” refer to a drug, substance, or molecule that binds to a protein, including but not limited to a receptor, and activates said protein triggering an intracellular signaling cascade, often leading to a physiological or pathological response. Generally, an agonist mimics the actions of a naturally occurring substance by binding the same proteins and triggering a similar intracellular response. In some embodiments, the agonist may show, for example, at least 60%, at least 70%, at least 80%, at least 90%, at least 91%, at least 92%, at least 93%, at least 94%, at least 95%, at least 96%, at least 97%, at least 98%, or at least 99% or greater identity relative to a reference parent or original substance.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

A “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

### Heparan Sulfate, TREM2, and Alzheimer's Disease

Heparan sulfate (HS) plays a significant role in numerous diseases associated with amyloid plaques, including but not limited to Alzheimer's Disease (AD), particularly in the formation of amyloid plaques and neurofibrillary tangles. HS interacts with amyloid-beta (Aβ) peptides, promoting their aggregation and formation of plaques. Additionally, HS interacts with tau protein, another key factor in AD pathology, also potentially influencing tau aggregation, plaque formation, and spread.

TREM2 is a protein expressed on microglia, the immune cells located in the brain, which play a crucial role in AD. TREM2 influences microglial functions including their ability to clear amyloid plaques and respond to inflammation. As such, TREM2 is essential for maintaining brain health by clearing debris, pathogens, and toxins from the brain. Further, mutations to TREM2 increases the risk of developing AD by preventing or slowing the clearance of amyloid-beta from the brain. Under normal conditions, TREM2 binds and internalizes amyloid-beta, where the amyloid-beta is destroyed by intracellular degradation pathways. Under pathological conditions, TREM2 is not able to bind amyloid-beta, thereby allowing amyloid-beta to bind other molecules of amyloid-beta and/or to heparan sulfate to increase plaque formation and promote AD pathogenesis. Although there has been a concerted effort to develop reliable therapeutic options to treat and prevent AD, there remains a need in the field to develop said therapeutic. The present disclosure presents the concept of targeting HS and/or TREM2 to reduce the aggregation and build-up of plaques in the brain of subject at risk of or suffering from AD.

### Methods of Inhibiting HS Biosynthesis

The present disclosure provides methods of treating and/or preventing a disease including, but not limited to AD, frontotemporal dementia, Parkinson's Disease, Nasu-Hakola diseases, a cancer, and atherosclerosis by administering inhibitors of heparan sulfate (HS) biosynthetic enzymes or inhibitors of HS-Triggering Receptor Expressed on Myeloid Cell-2 (HS-TREM2) complex formation.

In one aspect, disclosed herein is a method of treating a neurodegenerative disease, the method comprising administering to a subject in need thereof a pharmaceutical composition comprising at least one inhibitor of a heparan sulfate (HS) biosynthetic enzyme, wherein the at least one inhibitor decreases formation of a HS-Triggering Receptor Expressed on Myeloid Cells-2 (HS-TREM2) complex and decreases accumulation of Aβ relative to an untreated control.

A “neurodegenerative disease” is caused by the progressive loss of structure or function of neurons or glial cells, which make up the nervous system. These diseases include but are not limited to amyotrophic lateral sclerosis (ALS), multiple sclerosis, Parkinson's disease, Alzheimer's disease, Huntington's disease, and prion diseases. Neurodegenerative diseases can lead to cognitive and physical impairments, neuroinflammation (inflammation of the brain and spinal cord), and deterioration of brain and spinal cord tissues. In some embodiments, the neurodegenerative disease of any aspect disclosed herein includes, but is not limited to Alzheimer's Disease frontotemporal dementia, Parkinson's Disease, and Nasu-Hakola diseases.

In some embodiments, the inhibitor of a HS biosynthetic enzyme reduces, inhibits, decreases, and/or ameliorates the activity of one or more HS biosynthetic enzymes including, but not limited to N-deacetylase/N-sulfotransferases-1 (NDST1), Exostosin (EXT) glycosyltransferase, HS 6-O-sulfotransferase (HS6ST), glucuronyl C5-epimerase (Glce), or a combination thereof. In some embodiments, the inhibitor of HS biosynthesis will prevent synthesis of HS, and thereby lower the extracellular and/or intracellular concentration(s) of HS. In some embodiments, the method comprises administering a pharmaceutical composition comprising 1, 2, 3, 4, 5, or more inhibitors of a HS biosynthetic enzyme.

In some embodiments, the inhibitor of HS biosynthesis can lower HS concentration(s) by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% relative to a control. In some embodiments, when HS concentration(s) are lowered, binding of HS to amyloid is also lower. In some embodiments, the binding of HS to amyloid is lowered by 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% relative to a control HS.

In some embodiments, the at least one inhibitor of a HS biosynthetic enzyme includes, but is not limited to a peptide (including but not limited to a polypeptide, a peptidomimetic molecule, and a fusion polypeptide), an polynucleotide (including, but not limited to a DNA molecule, an RNA molecule, an oligonucleotide, an antisense oligonucleotide (ASO)), a small molecule, an immunoglobulin (including, but not limited to an antibody and a nanobody), an aptamer, a carbohydrate, a lipid, an organic molecule, an inorganic molecule, or any variant or combination thereof. In some embodiments, the at least one inhibitor of a HS biosynthetic enzyme comprises a “decoy” molecule. As used herein, a “decoy” molecule refers to any molecule that interacts with the HS biosynthetic enzyme in a similar manner as done by its natural ligand, however said “decoy” prevents, decreases, reduces, inhibits, or ameliorates normal physiological activity of HS and/or the HS biosynthetic enzyme.

In some embodiments, the at least one inhibitor of a HS biosynthetic enzyme comprises an inhibitor of NDST1, an inhibitor of a EXT glycosyltransferase, an inhibitor of HS6ST, an inhibitor of Glce, or a combination thereof. In some embodiments, the inhibitor of NDST1 comprises xyloside, an N-acetylglucosamine (GlcNAc) mimic molecule, or a derivative thereof. In some embodiments, the inhibitor of EXT includes, but is not limited to Manumycin A, Tipifarnib, Ketoconazole, and GW4869. It should be noted that an inhibitor of EXT may also be effective in inhibiting exosome biogenesis and release since EXT plays a crucial role in processes involving exosomes. In some embodiments, the EXT glycosyltransferase comprises an EXT1 or an EXT2. In some embodiments, the inhibitor of HS6ST includes, but is not limited to downstream products of HS biosynthesis (such as, for example hexasaccharides), peptide inhibitors comprising the amino acid sequences RGWRGEKIGN (SEQ ID NO: 17) and/or NMQALSMPVT (SEQ ID NO: 18). In some embodiments, the inhibitor of HS6ST comprises at least 60% sequence identity to SEQ ID NO: 17. In some embodiments, the inhibitor of HS6ST comprises 60%, 65%, 70%, 75%, 80%, 85%, 90%, 95%, 99%, or 100% sequence identity to SEQ ID NO: 17. In some embodiments, the inhibitor of HS6ST comprises at least 60% sequence identity to SEQ ID NO: 18. In some embodiments, the inhibitor of HS6ST comprises 60%, 65%, 70%, 75%, 80%, 85%, 90%, 95%, 99%, or 100% sequence identity to SEQ ID NO: 18. In some embodiments, the inhibitor of HS6ST comprises a small molecule inhibitor including but not limited to xylosides, rhodamine B, and genistein. In some embodiments, the inhibitor of HS6ST comprises a β-secretase inhibitor including but not limited to N-benzyloxcarbonyl-Valine-Leu-Leucinal (Z-VLL-CHO). In some embodiments, the inhibitor of HS6ST comprises an S-linked heparan sulfate analog, which are artificial sugar polymers that cannot be cleaved by heparinase. In some embodiments, the HS6ST comprises HS6ST1, HS6ST2, HS6ST3, or any variant thereof. In some embodiments, the inhibitor of Glce comprises a polysulfonated naphthylurea (including but not limited to Suramin). In some embodiments, the inhibitor of Glce comprises one or more metal ions including, but not limited to manganese (II) chloride).

In some embodiments, the inhibitor of HS biosynthesis of any preceding aspect is administered with one or more additional therapeutic agent(s) including, but not limited to antibiotics, antivirals, antineoplastic agents, analgesics, antipyretics, antidepressants, anti-cancer agents, antihistamines, anti-inflammatory agents, chemotherapeutic agents, diuretics, growth factors, hormones, non-steroidal anti-inflammatory agents, steroids, and vitamins.

The additional antibiotic comprises any one type of antibiotic including, but not limited to penicillins (including, but not limited to amoxicillin, clavulanate and amoxicillin, ampicillin, dicloxacillin, oxacillin, and penicillin V potassium), tetracyclins (including, but not limited to demeclocycline, doxycycline, eravacycline, minocycline, omadacycline, sarecycline, and tetracycline), cephalosporins (cefaclor, cefadroxil, cefdinir, cephalexin, cefprozil, cefepime, cefiderocol, cefotaxime, cefotetan, ceftaroline, cefazidme, ceftriaxone, and cefuroxime), quinolones (also referred to as fluoroquinolones include, but are not limited to ciprofloxacin, delafloxacin, levofloxacin, moxifloxacin, and gemifloxacin), lincomycins (including clindamycin and lincomycin), macrolides (including, but not limited to azithromycin, clarithromycin, erythromycin, and fidaxomicin (ketolide)), sulfonamides (including sulfamethoxazole and trimethoprim, and sulfasalazine), glycopeptides (including, but not limited to dalbavancin, oritavancin, telavancin, and vancomycin), aminoglycosides (including, but not limited to gentamicin, tobramycin, and amikacin), carbapenems (including, but not limited to imipenem and cilastatin, meropenem, and ertapenem), and topical antibiotics (including, but not limited to neomycin, bacitracin, polymyxin B, and praxomine) used alone or in combination.

In some embodiments, the anti-inflammatory compound is a non-steroidal anti-inflammatory compound including, but is not limited to aspirin, ibuprofen, ketoprofen, naproxen, steroids, glucocorticoids (including, but not limited to betamethasone, budesonide, dexamethasone, hydrocortisone, hydrocortisone acetate, methylprednisolone, prednisolone, prednisone, and triamcinolone), methotrexate, sulfasalazine, lefunomide, anti-Tumor Necrosis Factor (TNF) medications, cyclophosphamide, and mycophenolate. In some embodiments, the anesthetic includes, but is not limited to chloroprocaine, procaine, tetracaine, lidocaine, bupivacaine, ropivacaine, mepivacaine, and levobupivacaine. In some embodiments, the sedative include, but is not limited to barbiturates, benzodiazepines, nonbenzodiazepines hypnotics, antihistamines, muscle relaxants, opioids, and methaqualone, or derivatives thereof.

In some embodiments, the inhibitor of HS biosynthesis of any preceding aspect is administered with an anti-AD agent, including but not limited to a cholinesterase inhibitor (such as, for example donepezil, galantamine, memantine, rivastigmine, or any combination thereof), an anti-amyloid-beta agent (such as, for example BAN2401), or any combination thereof. In some embodiments, the inhibitor of HS biosynthesis of any preceding aspect is administered with an activator of phagocytosis including, but not limited to an antibody, a complement protein (such as, for example C3b), a lipid mediator (such as, for example leukotriene B4), cytokines (such as, for example IL-8 and granulocyte colony-stimulating factor (GCSF)), integrins, uridine-5′-diphosphate, calreticulin, histone proteins, and oxidized phospholipids (oxPLs).

In some embodiments, the inhibitor of HS biosynthesis of any preceding aspect may be administered in such amounts, time, and route deemed necessary in order to achieve the desired result. The exact amount of the inhibitor of HS biosynthesis of any preceding aspect will vary from subject to subject, depending on the species, age, and general condition of the subject, the severity of the neurodegeneration, the particular inhibitor of HS biosynthesis of any preceding aspect, its mode of administration, its mode of activity, and the like. The inhibitor of HS biosynthesis of any preceding aspect is preferably formulated in dosage unit form for ease of administration and uniformity of dosage. It will be understood, however, that the total daily usage of the inhibitor of HS biosynthesis of any preceding aspect will be decided by the attending physician within the scope of sound medical judgment. The specific therapeutically effective dose level for any particular subject will depend upon a variety of factors including the neurodegenerative disease being treated and the severity of the neurological dysfunction; the activity of the inhibitor of HS biosynthesis of any preceding aspect employed; the specific inhibitor of HS biosynthesis of any preceding aspect employed; the age, body weight, general health, sex and diet of the patient; the time of administration, route of administration, and rate of excretion of the specific inhibitor of HS biosynthesis of any preceding aspect employed; the duration of the treatment; drugs used in combination or coincidental with the specific inhibitor of HS biosynthesis of any preceding aspect employed; and like factors well known in the medical arts.

The inhibitor of HS biosynthesis of any preceding aspect may be administered by any route. In some embodiments, the inhibitor of HS biosynthesis of any preceding aspect is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the inhibitor of HS biosynthesis of any preceding aspect (e.g., its stability in the environment of the gastrointestinal tract), the condition of the subject (e.g., whether the subject is able to tolerate oral administration), etc.

The exact amount of inhibitor of HS biosynthesis of any preceding aspect required to achieve a therapeutically or prophylactically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In one aspect, disclosed herein is an inhibitor of HS biosynthesis of any preceding aspect and a pharmaceutically acceptable carrier selected from an excipient, a diluent, a salt, a buffer, a stabilizer, a lipid, an emulsion, a nanoparticle, and a cream. One or more active agents (e.g. a peptide (including but not limited to a polypeptide, a peptidomimetic molecule, and a fusion polypeptide), an polynucleotide (including, but not limited to a DNA molecule, an RNA molecule, an oligonucleotide, an antisense oligonucleotide (ASO)), a small molecule, an immunoglobulin (including, but not limited to an antibody and a nanobody), an aptamer, a carbohydrate, a lipid, an organic molecule, an inorganic molecule, or any variant or combination thereof) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4th Ed. N.Y. Wiley-Interscience.

In some embodiments, the inhibitor of HS biosynthesis of any preceding aspect is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the inhibitor of HS biosynthesis of any preceding aspect is administered daily. In some embodiments, the inhibitor of HS biosynthesis of any preceding aspect is administered every day, every 2 days, every 3 days, every 4 days, every 5 days, every 6 days, every 7 days, or more. In some embodiments, the inhibitor of HS biosynthesis of any preceding aspect is administered every week, every 2 weeks, every 3 weeks, every 4 weeks, or more. In some embodiments, the inhibitor of HS biosynthesis of any preceding aspect is administered every month, every 2 months, every 3 months, every 4 months, every 5 months, every 6 months, every 7 months, every 8 months, every 9 months, every 10 months, every 11 months, every 12 months, or more. In some embodiments, the inhibitor of HS biosynthesis of any preceding aspect is administered every year, every 2 years, every 3 years, every 4 years, every 5 years, or more.

In some embodiments, the neurodegenerative disease comprises Alzheimer's Disease (AD). In some embodiments, the method promotes phagocytosis and degradation of Aβ. In some embodiments, the inhibitor of NDST1 inhibits binding between HS and TREM2. In some embodiments, the inhibitor of NDST1 inhibits binding between HS and TREM2 by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to a control.

In some embodiments, the inhibitor of the EXT glycosyltransferase inhibits adding a sugar molecule to a heparan sulfate chain. In some embodiments, the inhibitor of the EXT of any preceding aspect inhibits adding a sugar molecule to a heparan sulfate chain by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to a control.

In some embodiments, the inhibitor of HS6ST or the inhibitor of Glce inhibits a modification to HS. In some embodiments, the modification comprises sulfonation to a glucosamine residue of HS. In some embodiments the inhibitor of the HS6ST of any preceding aspect inhibits sulfonation to a glucosamine residue of HS by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to a control.

In some embodiments, the modification comprises converting glucuronic acid (GlcA) to iduronic acid (IdoA). In some embodiments, the inhibitor of the Glce inhibits conversion of GlcA to IdoA by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to a control.

It should be understood that the method of any preceding aspect can be applied to other neurodegenerative, neoplastic, and/or cardiovascular diseases including but not limited to frontotemporal dementia, Parkinson's Disease, Nasu-Hakola diseases, a cancer, and atherosclerosis. Thus, said neurodegenerative, neoplastic, and/or cardiovascular diseases prevent, reduce, decrease, inhibit, and/or ameliorate the interaction between HS and TREM2.

### Methods of Activating TREM2 Activity

The present disclosure provides methods of treating and/or preventing a neurodegenerative disease by administering an agonist/activator of TREM2 activity. The present disclosure also provides methods of reducing accumulation of amyloid-β (Aβ).

In some aspects, disclosed herein is a method thereof a pharmaceutical composition comprising at least one agonist of a Triggering Receptor Expressed on Myeloid Cells-2 (TREM2), wherein the at least one agonist of TREM2 increases binding of amyloid-β (Aβ) to the TREM2 and decreases accumulation of Aβ relative to an untreated control.

A TREM2 agonist is a substance that activates the TREM2 receptor, primarily expressed on microglial cells in the brain. Since TREM2 is a key regulator of microglial cell functions and has been implicated in neurodegenerative diseases, including but not limited to Alzheimer's Disease, administering a TREM2 agonist can enhance TREM2 cell signaling and offer therapeutic benefits by modulating microglia responses to neurodegenerative diseases. It should be understood that the term “agonist” and “activator” can be used interchangeably.

In some embodiments, the agonist of TREM2 includes, but is not limited to a peptide (including but not limited to a polypeptide, a peptidomimetic molecule, and a fusion polypeptide), an polynucleotide (including, but not limited to a DNA molecule, an RNA molecule, an oligonucleotide, an antisense oligonucleotide (ASO)), a small molecule, an immunoglobulin (including, but not limited to an antibody and a nanobody), an aptamer, a carbohydrate, a lipid, an organic molecule, an inorganic molecule, or any variant or combination thereof. Non-limiting examples of TREM2 agonists are VG-3927, AL002, and dehyroervatamine (DHE).

In some embodiments, the TREM2 agonist of any preceding aspect increases, enhances, improves, and/or elevates TREM2 activity by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to a control.

In some embodiments, the agonist of TREM2 of any preceding aspect is administered with one or more additional therapeutic agent(s) including, but not limited to antibiotics, antivirals, antineoplastic agents, analgesics, antipyretics, antidepressants, anti-cancer agents, antihistamines, anti-inflammatory agents, chemotherapeutic agents, diuretics, growth factors, hormones, non-steroidal anti-inflammatory agents, steroids, and vitamins.

The additional antibiotic includes, but is not limited to penicillins (including, but not limited to amoxicillin, clavulanate and amoxicillin, ampicillin, dicloxacillin, oxacillin, and penicillin V potassium), tetracyclins (including, but not limited to demeclocycline, doxycycline, eravacycline, minocycline, omadacycline, sarecycline, and tetracycline), cephalosporins (cefaclor, cefadroxil, cefdinir, cephalexin, cefprozil, cefepime, cefiderocol, cefotaxime, cefotetan, ceftaroline, cefazidme, ceftriaxone, and cefuroxime), quinolones (also referred to as fluoroquinolones include, but are not limited to ciprofloxacin, delafloxacin, levofloxacin, moxifloxacin, and gemifloxacin), lincomycins (including clindamycin and lincomycin), macrolides (including, but not limited to azithromycin, clarithromycin, erythromycin, and fidaxomicin (ketolide)), sulfonamides (including sulfamethoxazole and trimethoprim, and sulfasalazine), glycopeptides (including, but not limited to dalbavancin, oritavancin, telavancin, and vancomycin), aminoglycosides (including, but not limited to gentamicin, tobramycin, and amikacin), carbapenems (including, but not limited to imipenem and cilastatin, meropenem, and ertapenem), and topical antibiotics (including, but not limited to neomycin, bacitracin, polymyxin B, and praxomine) used alone or in combination.

In some embodiments, the anti-inflammatory compound is a non-steroidal anti-inflammatory compound including, but is not limited to aspirin, ibuprofen, ketoprofen, naproxen, steroids, glucocorticoids (including, but not limited to betamethasone, budesonide, dexamethasone, hydrocortisone, hydrocortisone acetate, methylprednisolone, prednisolone, prednisone, and triamcinolone), methotrexate, sulfasalazine, lefunomide, anti-Tumor Necrosis Factor (TNF) medications, cyclophosphamide, and mycophenolate. In some embodiments, the anesthetic includes, but is not limited to chloroprocaine, procaine, tetracaine, lidocaine, bupivacaine, ropivacaine, mepivacaine, and levobupivacaine. In some embodiments, the sedative include, but is not limited to barbiturates, benzodiazepines, nonbenzodiazepines hypnotics, antihistamines, muscle relaxants, opioids, and methaqualone, or derivatives thereof.

In some embodiments, the agonist of TREM2 of any preceding aspect is administered with an anti-AD agent, including but not limited to a cholinesterase inhibitor (such as, for example donepezil, galantamine, memantine, rivastigmine, or any combination thereof), an anti-amyloid-beta agent (such as, for example BAN2401), or any combination thereof. In some embodiments, the agonist of TREM2 of any preceding aspect is administered with an activator of phagocytosis including, but not limited to an antibody, a complement protein (such as, for example C3b), a lipid mediator (such as, for example leukotriene B4), cytokines (such as, for example IL-8 and granulocyte colony-stimulating factor (GCSF)), integrins, uridine-5′-diphosphate, calreticulin, histone proteins, and oxidized phospholipids (oxPLs).

In some embodiments, the agonist of TREM2 of any preceding aspect may be administered in such amounts, time, and route deemed necessary in order to achieve the desired result. The exact amount of the agonist of TREM2 of any preceding aspect will vary from subject to subject, depending on the species, age, and general condition of the subject, the severity of the neurodegeneration, the particular agonist of TREM2 of any preceding aspect, its mode of administration, its mode of activity, and the like. The the agonist of TREM2 of any preceding aspect is preferably formulated in dosage unit form for ease of administration and uniformity of dosage. It will be understood, however, that the total daily usage of the the agonist of TREM2 of any preceding aspect will be decided by the attending physician within the scope of sound medical judgment. The specific therapeutically effective dose level for any particular subject will depend upon a variety of factors including the neurodegenerative disease being treated and the severity of the neurological dysfunction; the activity of the agonist of TREM2 of any preceding aspect employed; the specific agonist of TREM2 of any preceding aspect employed; the age, body weight, general health, sex and diet of the patient; the time of administration, route of administration, and rate of excretion of the specific agonist of TREM2 of any preceding aspect employed; the duration of the treatment; drugs used in combination or coincidental with the specific agonist of TREM2 of any preceding aspect employed; and like factors well known in the medical arts.

The agonist of TREM2 of any preceding aspect may be administered by any route. In some embodiments, the agonist of TREM2 of any preceding aspect is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the agonist of TREM2 of any preceding aspect (e.g., its stability in the environment of the gastrointestinal tract), the condition of the subject (e.g., whether the subject is able to tolerate oral administration), etc.

The exact amount of the agonist of TREM2 of any preceding aspect required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In one aspect, disclosed herein is an agonist of TREM2 of any preceding aspect and a pharmaceutically acceptable carrier selected from an excipient, a diluent, a salt, a buffer, a stabilizer, a lipid, an emulsion, a nanoparticle, and a cream. One or more active agents (e.g. a peptide (including but not limited to a polypeptide, a peptidomimetic molecule, and a fusion polypeptide), an polynucleotide (including, but not limited to a DNA molecule, an RNA molecule, an oligonucleotide, an antisense oligonucleotide (ASO)), a small molecule, an immunoglobulin (including, but not limited to an antibody and a nanobody), an aptamer, a carbohydrate, a lipid, an organic molecule, an inorganic molecule, or any variant or combination thereof) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4th Ed. N.Y. Wiley-Interscience.

In some embodiments, the agonist of TREM2 of any preceding aspect is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the agonist of TREM2 of any preceding aspect is administered daily. In some embodiments, the agonist of TREM2 of any preceding aspect is administered every day, every 2 days, every 3 days, every 4 days, every 5 days, every 6 days, every 7 days, or more. In some embodiments, the agonist of TREM2 any preceding aspect is administered every week, every 2 weeks, every 3 weeks, every 4 weeks, or more. In some embodiments, the agonist of TREM2 of any preceding aspect is administered every month, every 2 months, every 3 months, every 4 months, every 5 months, every 6 months, every 7 months, every 8 months, every 9 months, every 10 months, every 11 months, every 12 months, or more. In some embodiments, the agonist of TREM2 of any preceding aspect is administered every year, every 2 years, every 3 years, every 4 years, every 5 years, or more.

In some embodiments, the neurodegenerative disease comprises Alzheimer's Disease (AD). In some embodiments, the method promotes phagocytosis and degradation of Aβ.

In some aspects, disclosed herein is a method of reducing accumulation of Aβ, the method comprising contacting the cell with at least one inhibitor of a heparan sulfate (HS) biosynthetic enzyme, at least one agonist of a Triggering Receptor Expressed on Myeloid Cells-2 (TREM2), or a combination thereof, wherein the method decreases formation of a HS-TREM2 complex and increases binding of Aβ to the TREM2.

In some embodiments, the method of reducing accumulation of Aβ comprises administering the at least one inhibitor of an HS biosynthetic enzyme of any preceding aspect. In some embodiments the method of reducing accumulations of Aβ comprises administering the at least one agonist of TREM2 of any preceding aspect. In some embodiments, the method of reducing accumulation of Aβ lowers the concentration of Aβ by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to a control.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: TREM2 on Microglia Cell Surface Binds to and Forms Functional Binary Complexes with Heparan Sulfate Modified with 6-O-Sulfation and Iduronic Acid

The Triggering Receptor Expressed on Myeloid Cells-2 (TREM2), a pivotal innate immune receptor, orchestrates functions such as inflammatory responses, phagocytosis, cell survival, and neuroprotection. TREM2 variants R47H and R62H have been associated with Alzheimer's disease, yet the underlying mechanisms remain elusive. TREM2 binds to heparan sulfate (HS) and variants R47H and R62H exhibit reduced affinity for HS. Building upon this groundwork, the present disclosure delves into the interplay between TREM2 and HS and its impact on microglial function. TREM2's binds to cell surface HS and interacts with HS, forming HS-TREM2 binary complexes on microglia cell surfaces. Employing various biochemical techniques, including Surface Plasmon Resonance, low molecular weight HS microarray screening, and serial HS mutant cell surface binding assays, the present disclosure demonstrates TREM2's robust affinity for HS, and the effective binding requires a minimum HS size of approximately 10 saccharide units. Notably, TREM2 selectively binds specific HS structures, with 6-O-sulfation and, to a lesser extent, the iduronic acid residue playing crucial roles. N-sulfation and 2-O-sulfation are dispensable for this interaction. Furthermore, 6-O-sulfation is essential for HS-TREM2 ternary complex formation on the microglial cell surface, and HS and its 6-O-sulfation are necessary for TREM2-mediated ApoE3 uptake in microglia. By delineating the interaction between HS and TREM2 on the microglial cell surface and demonstrating its role in facilitating TREM2-mediated ApoE uptake by microglia, these findings provide valuable insights that can inform targeted interventions for modulating microglial functions in Alzheimer's disease.

Triggering receptor expressed on myeloid cells-2 (TREM2), an innate immune receptor, is expressed in various myeloid cells, including dendritic cells, resident macrophages such as osteoclasts and microglia, infiltrating and inflammatory macrophages, and cerebrospinal fluid monocytes. TREM2 is a type-one receptor featuring an extracellular V-type Ig domain, a short stalk, a transmembrane domain that associates with the adaptor protein DAP12 for trafficking and signaling, and a cytoplasmic tail. TREM2 plays a vital role in regulating inflammatory responses, phagocytosis, cell survival, and neuroprotection. Identified variants within the TREM2 gene have been implicated as risk factors for multiple neurodegenerative conditions. Specifically, the R47H and R62H variants are associated with Alzheimer's disease (AD), while the T66M variant is linked to Nasu-Hakola disease. Although the exact mechanisms by which TREM2 mutations contribute to the disease pathogenesis are not fully understood, accumulating evidence shows their involvement in dysregulating microglia function, impairing phagocytosis, altering inflammatory responses, and compromising neuronal support and survival.

A comprehensive analysis on wildtype and variant TREM2 proteins elucidated their structural and functional characteristics. Examination of the TREM2 crystal structure at 3.1 Å revealed distinct patterns: mutations associated with the Nasu-Hakola disease were buried within the protein, while variants linked to AD were located on the protein's surface. This distinction implies that these mutations elicit divergent effects on TREM2 function. By utilizing a combination of biophysical and cellular methodologies, it was discerned that Nasu-Hakola mutations significantly impact protein stability, leading to a notable reduction in folded TREM2 surface expression. Conversely, AD risk variants specifically influence the binding affinity of TREM2 to its ligand, heparan sulfate (HS). This observation not only unveils distinct molecular mechanisms underlying the variants associated with Nasu-Hakola and AD pathogenesis but also hints at a regulatory role for HS in modulating TREM2 signaling and associated functions in AD.

HS is a linear polysaccharide in the glycosaminoglycan (GAG) family. During biosynthesis, HS covalently binds to protein cores, forming HS proteoglycans (HSPGs). Depending on the protein cores, HSPGs are expressed on cell surfaces and in the extracellular matrix. Their HS moieties interact with many protein ligands, including growth factors, growth factor receptors, morphogens, cytokines, and matrix proteins. These interactions modulate various physiological and pathological processes, such as development, leukocyte trafficking, tumorigenesis, and lipid metabolism. Biochemical studies have revealed that HS generally interacts with protein ligands through unique binding sites. These sites consist of relatively small segments of variably sulfated glucosamine and uronic acid epimer residues, including N-, 2-, 6- and 3-O-sulfation (NS, 2S, 6S, and 3S, correspondingly), as well as epimers of glucuronic acid (GlcA) and iduronic acid (IdoA) arranged in specific patterns. Herein, it is confirmed that TREM2 binds to the cell surface in an HS-dependent manner and further demonstrated that TREM2 interacts in cis with HS, forming TREM2-HS binary complexes on the microglial cell surface. Biochemical interaction and cell surface binding analyses revealed that TREM2 selectively binds specific HS structures, with 6-O-sulfation and, to a lesser extent, the iduronic acid residue playing crucial roles. N-sulfation and 2-O-sulfation are dispensable for this interaction. Functional studies further show that HS and 6S facilitate TREM2-mediated ApoE3 uptake in microglia. These findings illuminate the critical role and structure of HS in regulating TREM2 function within microglia, with implications for better understanding neurodegenerative diseases and informing targeted interventions for disease treatment.

### Results

TREM2 interacts with HS to form TREM2-HS binary complexes on the microglia cell surface.

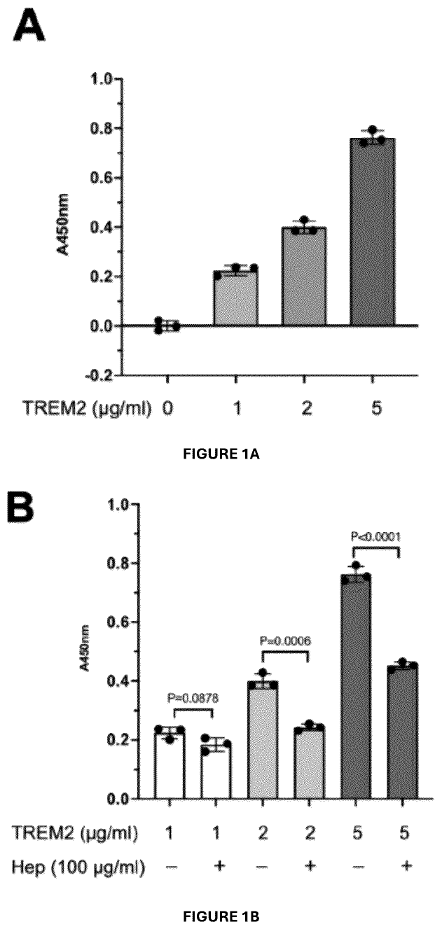

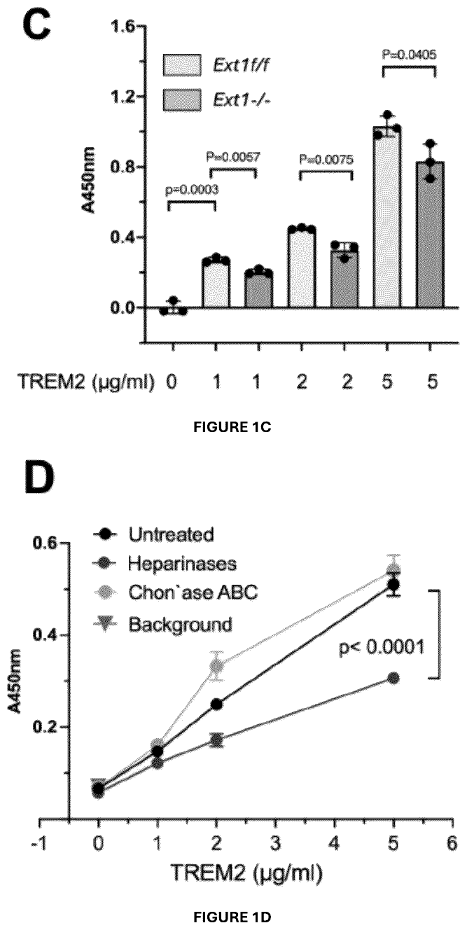

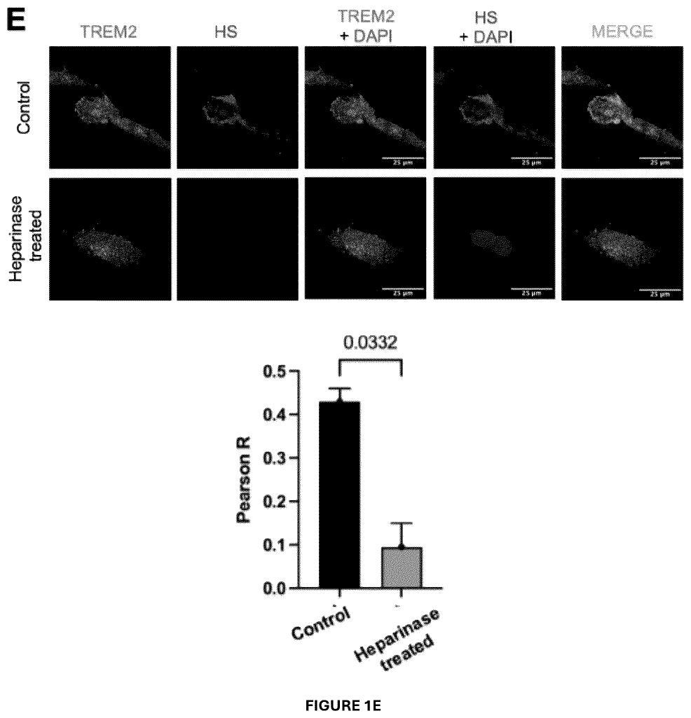

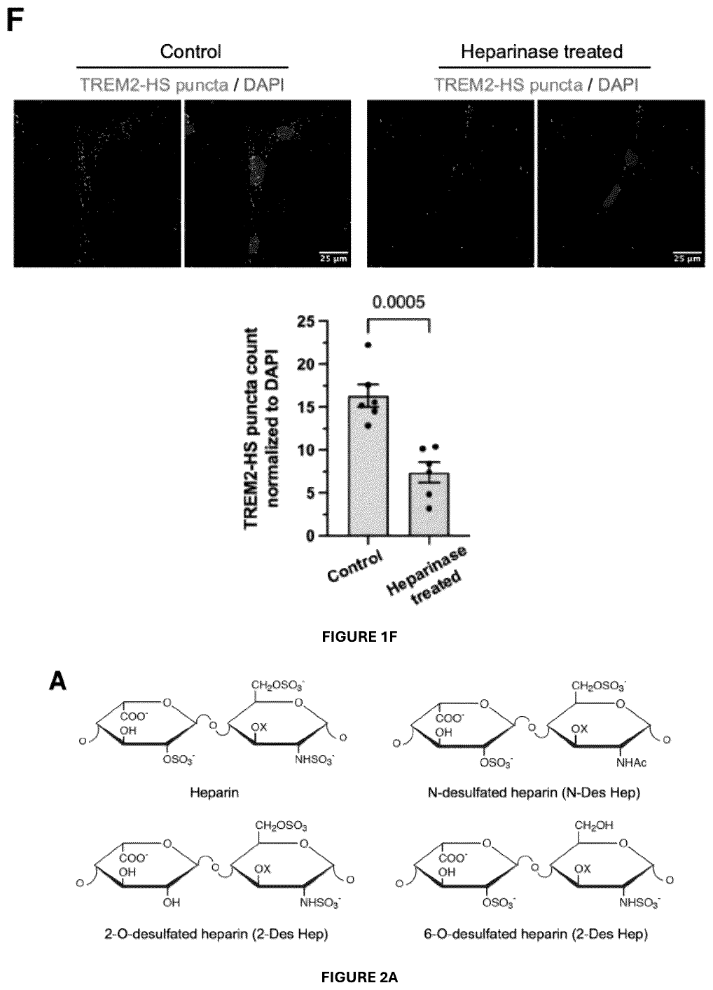

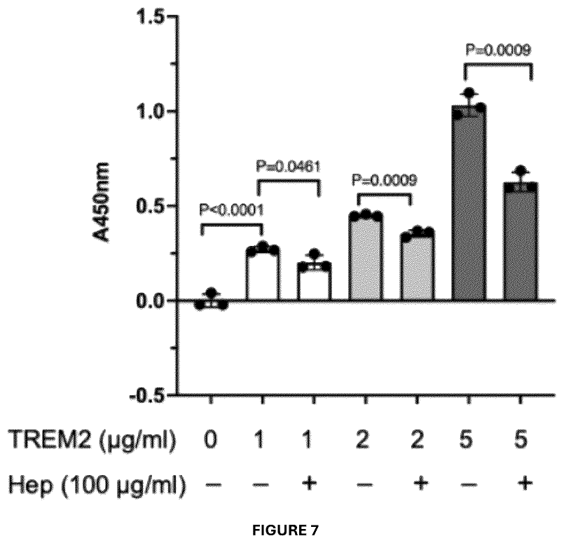

Previously, CHO, THP-1, and N2A cells were examined and it was observed that the recombinant human and mouse TREM2 ectodomain binds to the cell surface in an HS-dependent manner. HS is abundantly expressed on the endothelial cell surface, and endothelial cells are commonly used models to determine the HS-depend function of molecules such as growth factors and adhesion molecules. Previously, a mouse lung endothelial cell (MLEC) HS mutant library was studied to systemically analyze HS structure-function relationships. To determine HS-dependent TREM2 binding on the endothelial cell surface, wildtype MLEC lines were tested from this library. Two wildtype MLEC lines were examined: one derived from the conditionally targeted HS N-deacetylase N-sulfotransferases-1 (Ndst1^{f/f}) mice and another from the conditionally targeted exostosin-1 (Ext1^{f/f}) mice. Additionally, an Ext1-deficient (Ext1^{-/-}) MLECs derived from Ext1^{f/f} cells after the targeted Ext1 alleles were deleted with Adeno-Cre were examined, resulting in deficient HS expression. TREM2 concentration-dependent binding to the wildtype cell surface was observed (FIG. 1A). This binding was attenuated by pre-mixing with heparin, a highly sulfated form of HS, which competitively inhibited TREM2 binding to the wildtype MLEC surface HS (FIG. 1B and FIG. 7), or by Ext1 deletion, which abolished HS expression (FIG. 1C). In parallel, the cells were treated with heparinases I and III or chondroitinase ABC (chon ase ABC) and found that heparinases, but not chon ase ABC, reduced cell surface TREM2 binding (FIG. 1D). These data confirm that cell surface TREM2 binding is HS-dependent.

Next, it was investigated whether cell surface-expressed TREM2 binds to the cell surface HS using human microglia C20 cells. Immunostaining revealed high expression of both TREM2 and HS on the C20 cell surface (FIG. 1E). A significant portion of TREM2 co-localized with HS, quantified by high Pearson's R correlation coefficient (R=0.43). This co-localization was attenuated in heparinase-treated cells (R=0.095). A proximity ligation assay (PLA) was also applied to determine whether TREM2 and HS form binary complexes on the C20 cell surface. After staining with primary rabbit anti-human TREM2 and primary mouse anti-HS 104 antibodies, the cells were incubated with DuoLink rabbit plus and mouse minus probes, followed by ligation and amplification steps. An abundance of TREM2-HS binary complexes was observed, indicated by red dots on the C20 cell surface (FIG. 1F). Notably, TREM2-HS complex formation was dramatically reduced when the cell surface HS was degraded by heparinase treatment before PLA staining. The co-localization and PLA results demonstrate that TREM2 interacts with HS in cis to form TREM2-HS binary complexes on the microglial cell surface, showing that HS may modulate TREM2 signaling and microglia functions.

TREM2 Exhibits High-Affinity Binding to Heparin which is Dependent on the Size of the HS Oligosaccharide

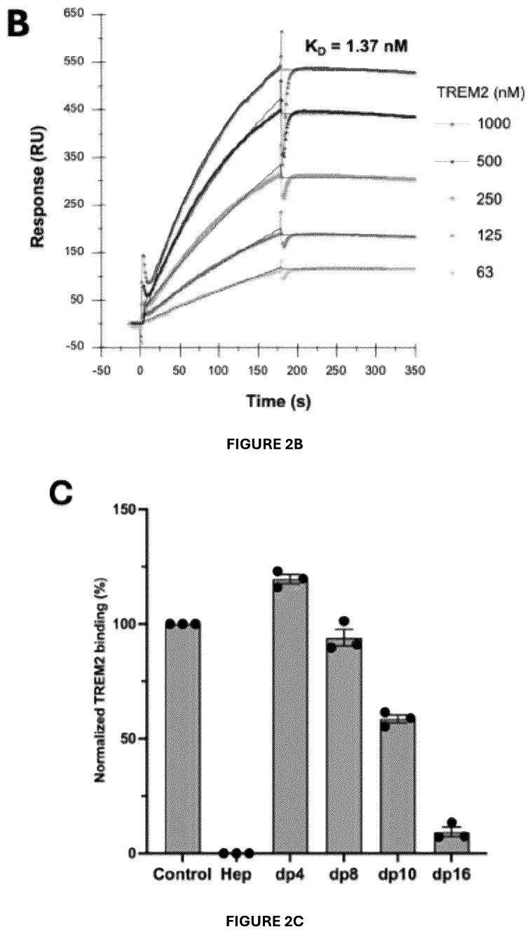

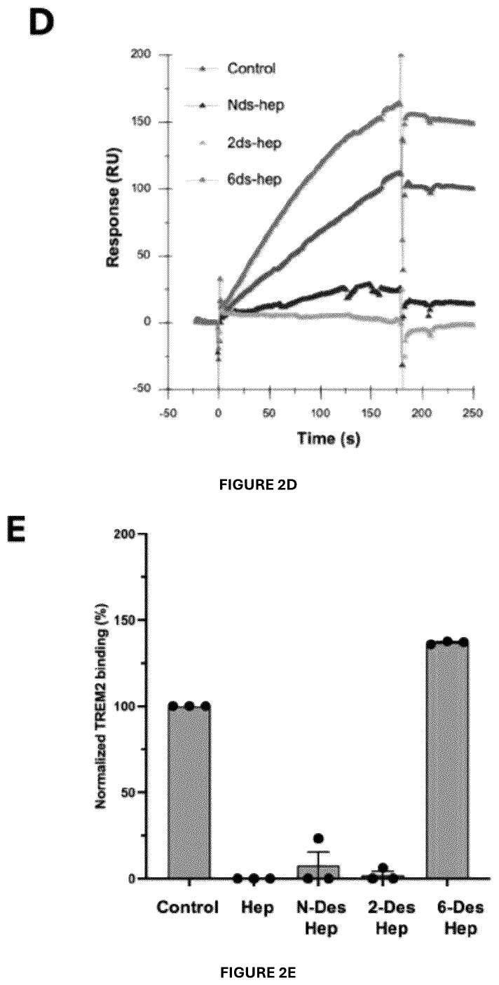

In previous investigations, a conserved surface site on TREM2 was observed that contains residues associated with AD risk variants (R47H and R62H) and a protective variant (T96K) (7). This site also binds to HS, with R47H and R62H mutations decreasing HS binding, while T96K mutation enhances HS binding. These findings underscore the critical role of altered TREM2-HS interaction in modulating AD risk. However, the TREM2 binding affinity and the specific structure of HS molecule involved in this interaction remain unknown. To investigate TREM2-HS interaction in greater detail, heparin, which is a highly sulfated analog of HS commonly used as a surrogate, was utilized. In surface plasmon resonance (SPR) experiments, biotinylated streptavidin sensor chips were immobilized and sensorgrams depicting the interaction between TREM2 and chip surface-immobilized heparin were generated by injecting TREM2 at various concentrations (1000, 500, 250, 125, and 63 nM) (FIGS. 2A, 2B). The sensorgrams obtained at different TREM2 concentrations were globally fitted to the 1:1 Langmuir binding model, revealing a dissociation constant (K_{D}) of 1.37±1.20×10⁻⁹ M, that indicated a very high binding affinity between TREM2 and heparin. Furthermore, solution/surface competitive TREM2 binding SPR analysis was conducted to determine the minimum heparin size required to bind to TREM2. Heparin-derived oligosaccharides of various sizes, ranging from dp4 to dp16, were examined at 1000 nM in the solution phase and premixed with TREM2 at 250 nM. Heparin completely inhibited TREM2 binding to surface-immobilized heparin (FIG. 2C). While oligosaccharides dp4 and dp8 showed no competition, significant competition was observed for the dp10 oligosaccharide (45% inhibition), and dp16 exhibited potent inhibition (93%). These findings indicate that the binding of HS to TREM2 requires a minimum heparin size of approximately 10 saccharide units.

6-O-Sulfation and, to a Lesser Extent, Iduronic Acid, but not N- and 2-O-Sulfation, are Required for HS to Bind TREM2

HS contains common sulfation modifications important for binding protein, including N-sulfation (NS), 2-O-sulfation (2S), and 6-O-sulfation (6S), along with the less common 3-O-sulfation (3S). To pinpoint the essential sulfation modification for HS binding to TREM2, an SPR competition assay in the presence of native heparin or chemically modified heparin variants, including N-desulfated heparin (N-Des Hep), 2-O-desulfated heparin (2-Des Hep), and 6-O-desulfated heparin (6-Des Hep) was used. While heparin, N-Des Hep, and 2-Des Hep displayed potent and similar inhibitory activities, 6-Des Hep showed none, indicating the necessity of 6S for HS binding to TREM2 (FIGS. 2D, 2E).

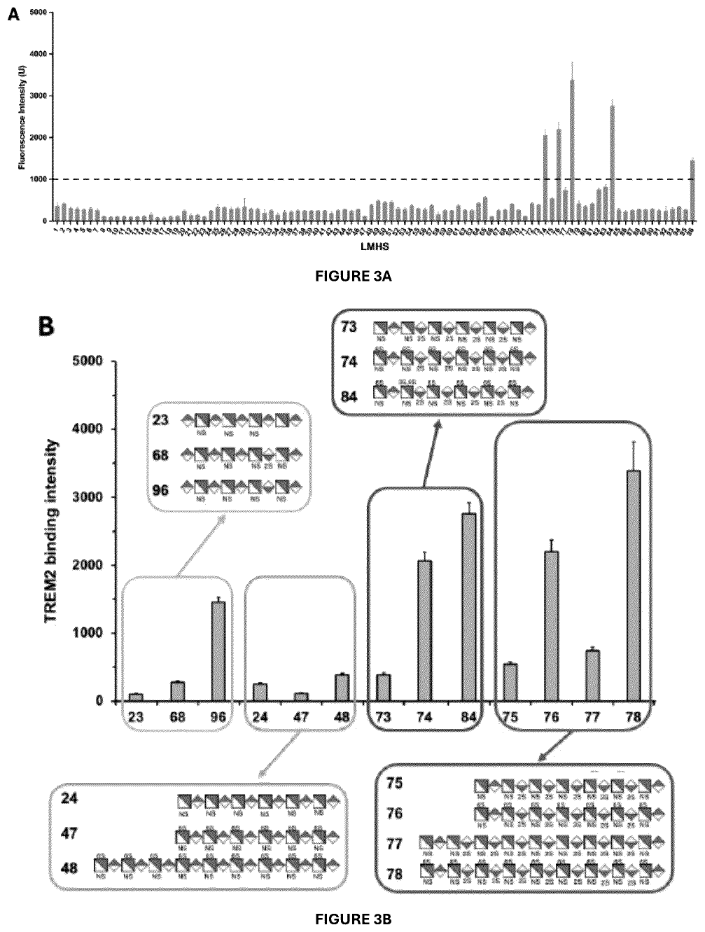

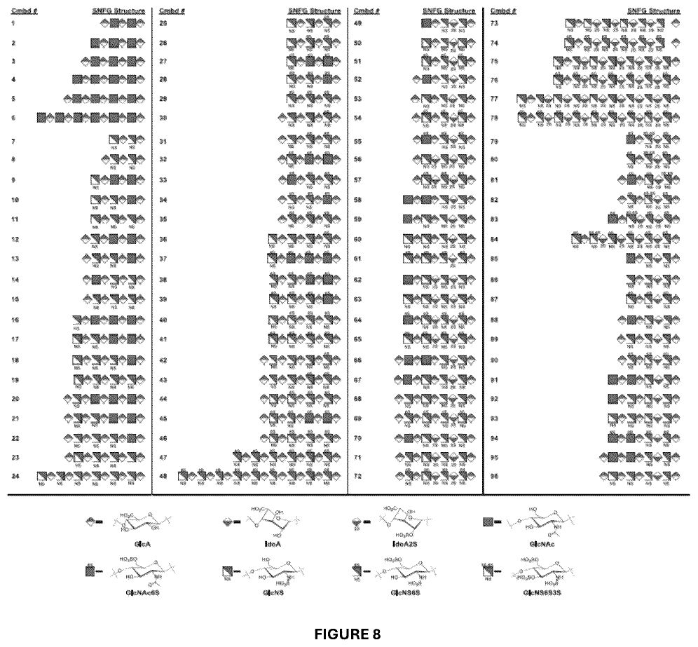

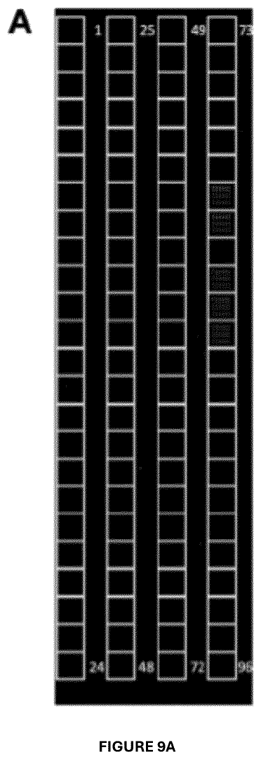

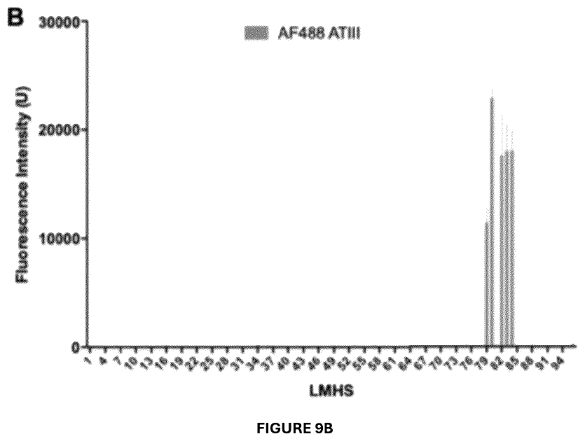

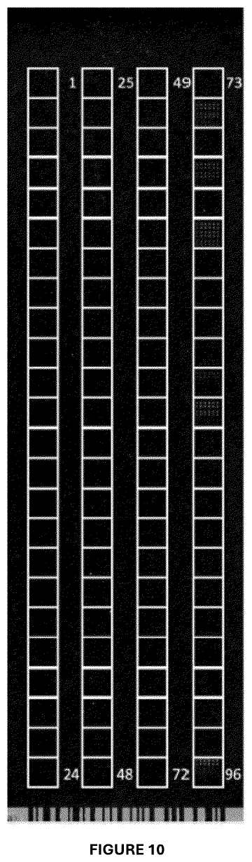

Considering the structural heterogeneity of heparin and its derivatives, a low molecular weight (LMW) HS microarray was employed to elucidate the specific HS structure needed for TREM2 binding. This microarray facilitates the simultaneous investigation of protein-HS interactions across various HS structures with defined chain lengths, sequence, and sulfation patterns (FIG. 8). Anti-thrombin binding to known HS structures within the microarray served as an analysis quality control (FIGS. 9A and 9B). Among the 96 HS oligosaccharides probed, only 5 exhibited binding to TREM2, including HS oligosaccharides #74, #76, #78, #84, and #96 (FIG. 3A and FIG. 10 and Table 1). #78 was the strongest binder and had much better binding to TREM2 than #77. #78 differs from #77 only by additional 6S modifications (FIG. 3B and Table 1), affirming a clear dependence on 6S for TREM2-HS interaction observed by SPR competition analysis. Similar trends for 6S dependence were observed in the comparison of #84 vs #73 and #76 vs #75. The TREM2-bound HS oligosaccharides ranged in size from 9 to 18 saccharide residues, affirming a minimum requirement of approximately 10 monosaccharide units for TREM2 binding (FIG. 3B).

Intriguingly, the presence of a 3S modification in HS #84, compared to HS #74, significantly enhanced TREM2 binding, showing a contributory role of 3S. This is reminiscent of 3S dependence of HS binding of other proteins associated with AD, such as tau and ApoE.

HS #23, #68, and #96 are all 9-mers, yet only HS #96 exhibited weak but significant binding to TREM2, whereas the other two did not bind. Comparing HS #23 to HS #96 reveals a substitution of GlcA with IdoA, suggesting a role of IdoA in TREM2 binding. However, in HS #68, an additional 2S modification on the IdoA, compared to HS #96, implies a blocking effect on TREM2 binding. Interestingly, TREM2-binding HS oligomers, such as #74, 76, 78, and 84, contain IdoA-2S residues, suggesting that the presence of 6S in longer HS chains may overcome the inhibitory effect of 2S.

Moreover, other HS oligomers that are larger and have higher overall sulfation levels, such as HS #24, #47, and #48 (ranging from 12 to 18 mers with 6 to 18 sulfates), did not bind TREM2. This contrasts with observations for HS #96, a 9-mer with only 4 sulfates, and HS #74, a 12-mer with 16 sulfates, both exhibited binding to TREM2. Therefore, the interaction between HS and TREM2 is not contingent upon the overall sulfation level but rather necessitates a specific HS structural motif.

In summary, the SPR analyses involving chemically modified heparins, alongside LMW HS microarray analysis, indicate that TREM2 binds selectively to a particular HS structure, with 6S being indispensable for HS-TREM2 binding. Additionally, the findings show the contributory roles of other modifications in the binding, including IdoA residue and 3S

### Deficiencies of Hs6st, Ndst1 and Glce, but not Hs2st and Hs3st1 Attenuate Cell Surface HS Binding of TREM2

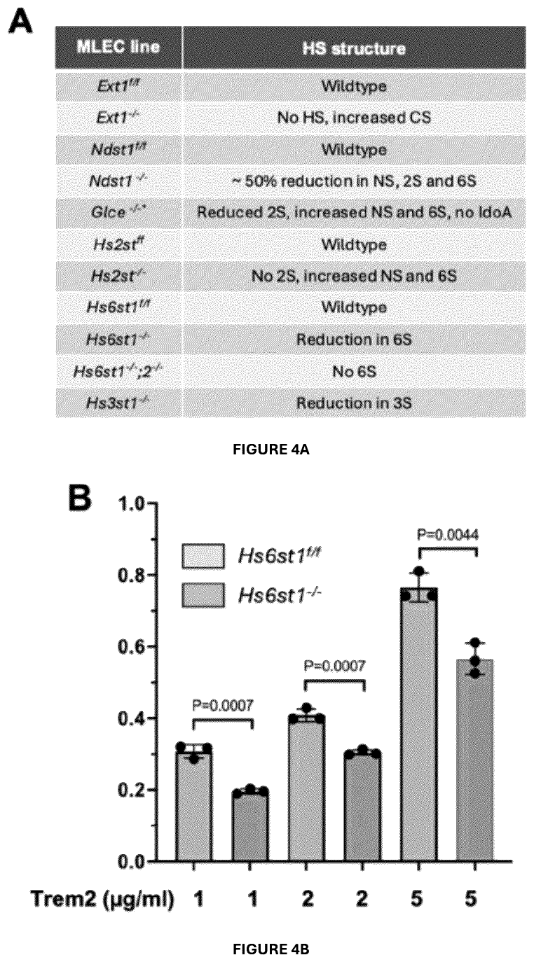

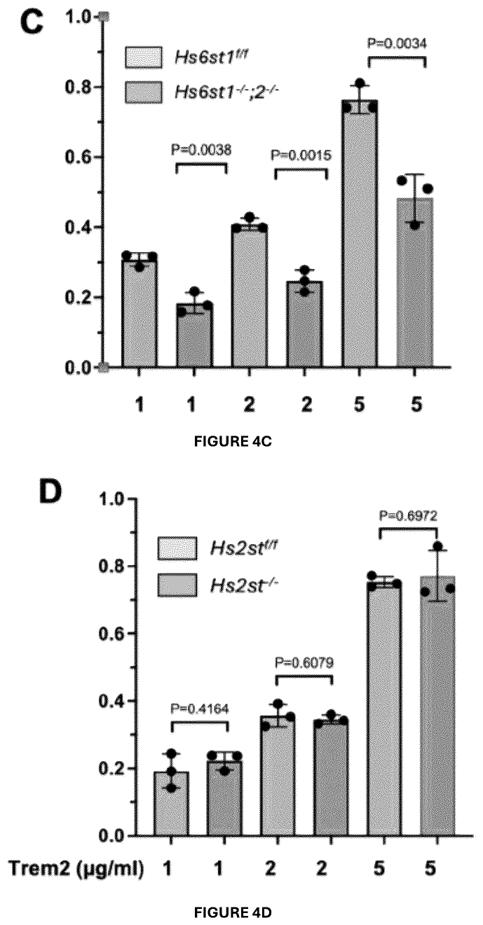

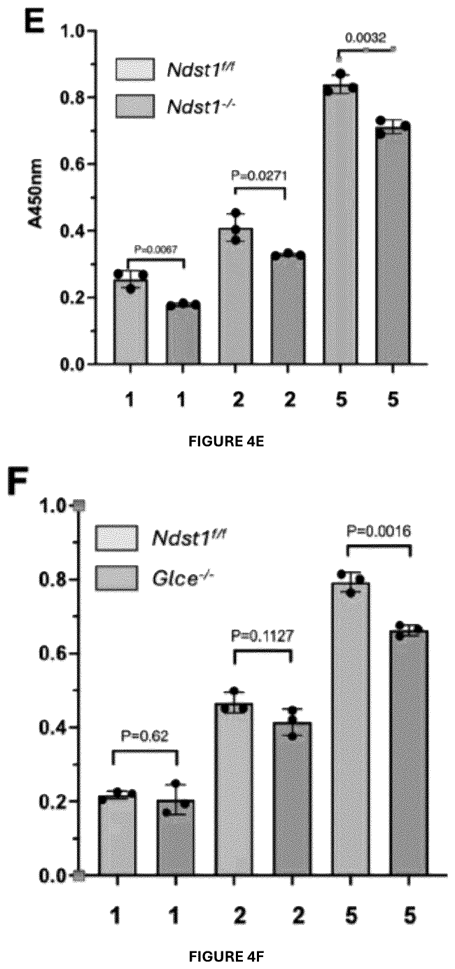

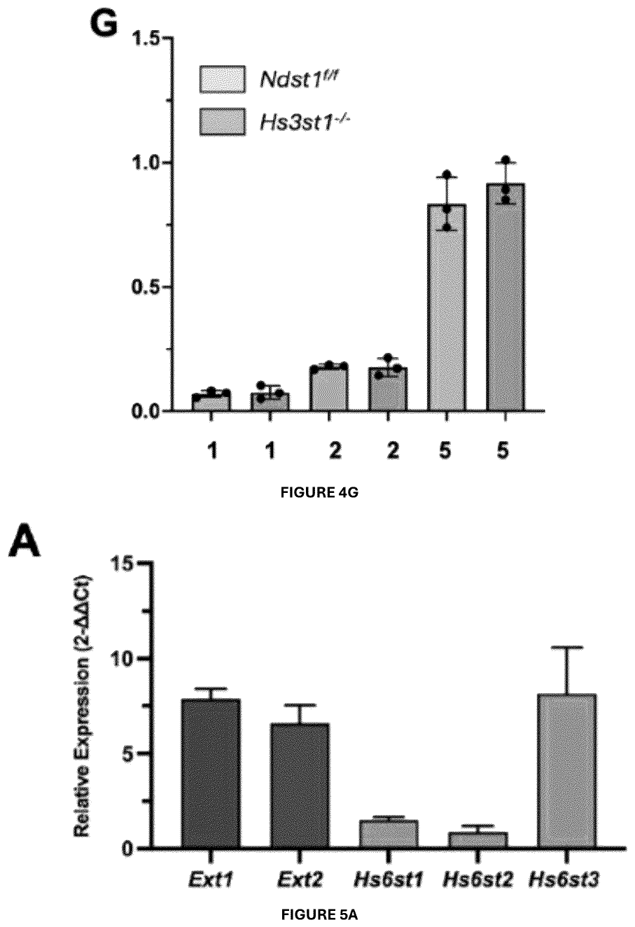

The biosynthesis of HS occurs within the Golgi apparatus and involves a series of enzymes from multiple families. The heterodimer Ext1/2 acts as a co-polymerase, elongating the HS chain by sequentially adding GlcA and N-acetylglucosamine (GlcNAc) residues. As the HS chain grows, it undergoes various modifications, including NS of GlcNAc by Ndst1-4, conversion of GlcA to IdoA by glucuronyl C5-epimerase (Glce) and, 2S of IdoA, and less frequently of GlcA, by HS 2-O-sulfotransferase (Hs2st), as well as 6S and 3S of N-sulfated GlcNAc (GlcNS) by HS 6-O-sulfotransferase1-3 (Hs6st1-3) and HS 3-O-sulfotransferase1-6 (Hs3st1-6), respectively. Once synthesized, HS may undergo further modifications on the cell surface and in the extracellular matrix by 6-O-endosulfatases (Sulf1-2) enzymes, which remove 6S from GlcNS. The abundance and structure of HS expression are intricately regulated by the concerted actions of the enzymes responsible for its biosynthesis and remodeling. Previously, it was established that a comprehensive HS mutant MLEC library using Cre-LoxP gene targeting and CRISPR-Cas9 approaches. This library enabled us to precisely assess the contribution of various HS modifications to TREM2 binding in a cellular context. To elucidate the specific roles of NS, 2S, 6S,3S, and epimerization in HS binding to TREM2, the cell surface binding of TREM2 to HS mutant cells, including Ndst1^{-/-}, Hs2st^{-/-}, Hs6st1^{-/-}, Hs6st1^{-/-}; 2^{-/-}, Hs3st1^{-/-} and Glce^{-/-} MLECs, along with their corresponding isogenic wildtype controls was systematically analyzed (FIG. 4A). Cells were incubated with TREM2 at concentrations of 1, 2, and 5 μg/ml, and observed reduced cell surface TREM2 binding on the Hs6st1^{-/-}, and Hs6st1^{-/-}; 2^{-/-} cell surface across all examined TREM2 concentrations (FIGS. 4B and 4C). Since Hs6st1^{-/-}, and Hs6st1^{-/-}; 2^{-/-} cells partially or completely lack 6S, respectively, the diminished cell surface binding reiterates that 6S is essential for cell surface HS binding to TREM2. Conversely, Hs2st^{-/-} cells, which lack 2S and have compensatory increases in NS and 6S, did not show altered TREM2 binding (FIG. 4D), highlighting the dispensable nature of 2S in this interaction. Furthermore, reduced TREM2 binding on Ndst1^{-/-} cell surfaces was observed (FIG. 4E), likely due to decreased 6S content in Ndst1^{-/-} HS, since N-desulfation did not affect heparin binding to TREM2, and 2S is dispensable for cellular HS to bind TREM2. The Glce^{-/-} and Hs3st1^{-/-} MLECs were generated from the wildtype Ndst1^{f/f} cells through CRISPR-Cas9-mediated gene deletion. TREM2 at a concentration of 5 μg/ml showed reduced cell surface binding on Glce-cells compared to their isogenic Ndst1^{f/f} counterparts (FIG. 4F). Glce HS exhibits decreased 2S and increased NS and 6S, and lacking IdoA compared to its isogenic wildtype Ndst1^{f/f} HS. These structural features show that the reduced TREM2 binding is due to the deficiency of IdoA, considering that NS and 2S did not affect TREM2 binding even though 6S was increased. Hs3st1^{-/-} MLECs exhibited unchanged cell surface TREM2 binding compared to their isogenic Ndst1^{f/f} counterparts (FIG. 4G). Since Hs3st1 deletion only partially reduces 3S and different Hs3sts may generate distinct 3S-modified HS structures, the contribution of 3S produced by other Hs3sts to TREM2 binding cannot be excluded. Overall, the serial HS mutant cell studies revealed that 6S and, to a lesser extent, IdoA are required for HS to bind TREM2 on the cell surface, while NS and 2S play nonessential roles in this process.

### Deficiencies in Hs6Sts Disrupt Microglia Cell Surface TREM2-HS Binary Complex Formation

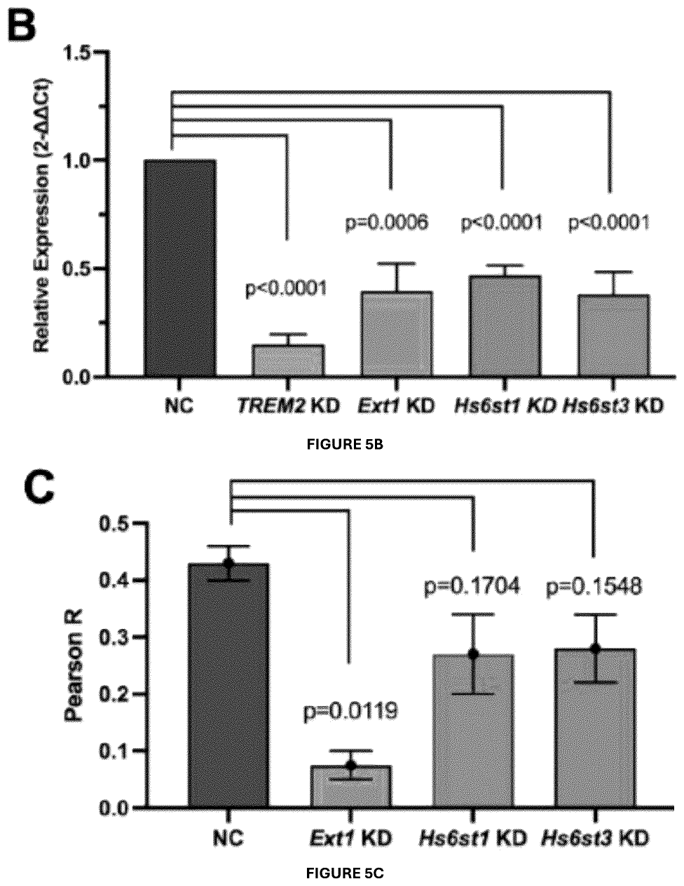

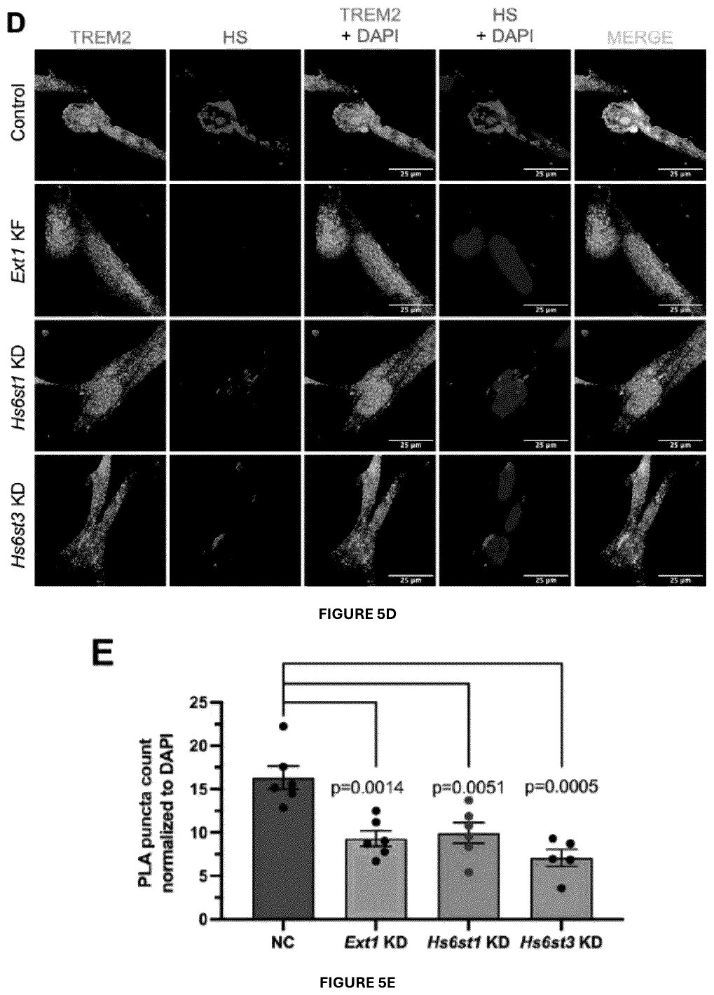

To determine the requirement of 6S for HS binding to microglial cell surface-expressed TREM2, HS gene expression was initially profiled in C20 cells. C20 cells express Ext1 and Ext2 (FIG. 5A). Among the Hs6st family, C20 cells exhibit high expression of Hs6st3, a moderate expression of Hs6st1, and a low expression of Hs6st2 (FIG. 5A). Ext1, Hs6st1 and Hs6st3 was knocked down using specific siRNAs (FIG. 5B). Immunofluorescence staining revealed high expression levels of TREM2 and HS on the cell surface, with significant colocalization between them (Pearson R value=0.420) (FIGS. 5C and 5D). The HS expression and TREM2 colocalization were diminished in Ext/knockdown C20 cells, validating the efficiency of the analysis. Notably, the colocalization of HS and TREM2 showed a decreasing trend in C20 cells with individual knockdowns of either Hs6st1 or Hs6st3. Additionally, PLA using anti-HS 10E4 and anti-human TREM2 antibodies revealed abundant TREM2-HS binary complex puncta in empty vector-transfected C20 cells (FIGS. 5E and 5F). Importantly, the number of these puncta significantly decreased upon individual knockdowns of Ext1, Hs6st1, or Hs6st3. These findings confirm that HS binds TREM2 in cis on the microglia cell surface and highlight the essential role of HS-6S in this binding and binary complex formation process.

### Deficiencies in Ext1 and Hs6st Disrupt Microglial Uptake of ApoE3 Protein

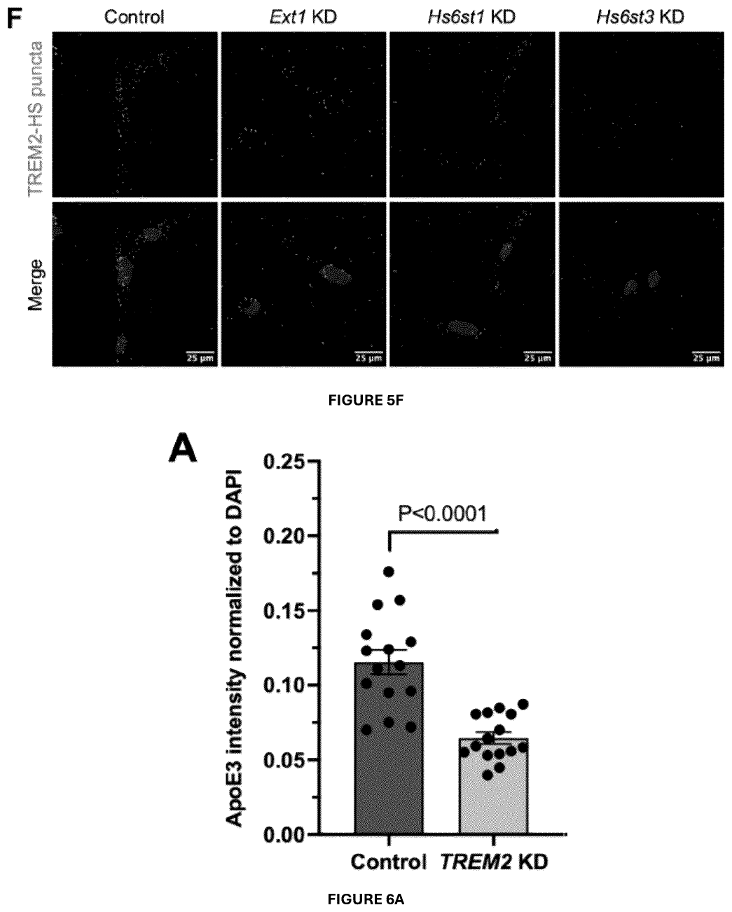

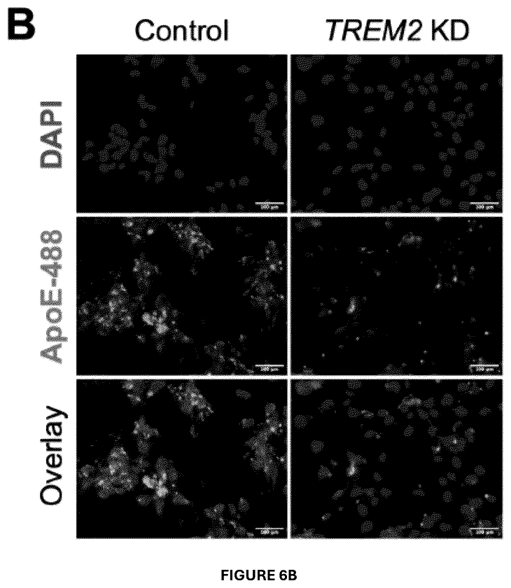

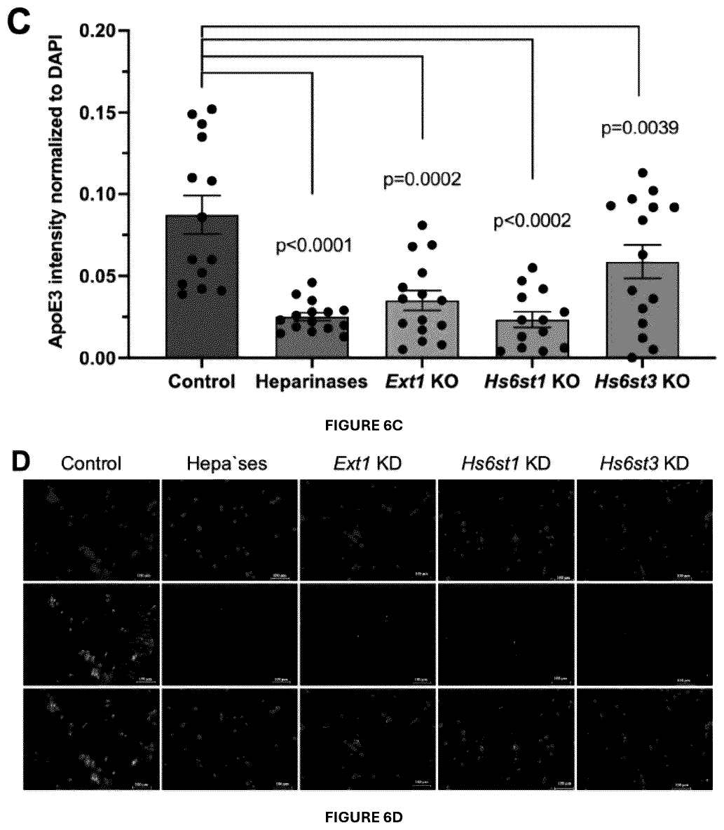

ApoE is a 299-amino acid protein that serves as a major cholesterol carrier in circulation and the sole cholesterol transporter in the brain. Genome-wide association studies (GWAS) and whole-exome sequencing have identified over 30 risk loci associated with AD, including ApoE and TREM2. TREM2 has been demonstrated to bind to ApoE and act as its receptor, showing that the ApoE-TREM2 interaction may modulate AD pathogenesis. TREM2 mediates ApoE uptake in microglia. This was confirmed by demonstrating that TREM2 knockdown attenuated the uptake of human ApoE3 conjugated with Alexa Fluor 488 (ApoE3-488) by C20 cells (FIGS. 5B, 6A, and 6B). Furthermore, ApoE3-488 uptake by C20 cells was significantly reduced upon individual knockdown of Ext1, Hs6st1, or Hs6st3, indicating that HS and HS-6S facilitate TREM2-mediated ApoE3 uptake in microglia (FIGS. 6C and 6D). It was previously demonstrated that ApoE3 binds to HS. Therefore, the new findings here show that HS may act as a co-receptor, simultaneously interacting with ApoE3 and TREM2 on the microglial cell surface to facilitate ApoE3 uptake.

### Discussion

Recent studies on human populations have highlighted the critical role of HS in AD. The loss-of-HS binding ApoE3 Christchurch variant (R136S) confers protective effects in autosomal dominant AD patients with PSEN1 mutations and in AD mouse models. Conversely, the RELN variant (H3447R), characterized by heightened HS binding, also provides protection in autosomal dominant AD patients and mouse models. These protective effects associated with the APOE3 Christchurch variant and the RELN (H3447R) variant are believed to arise from alterations in HS binding. Meanwhile, recent studies have emphasized the importance of TREM2 and neuroinflammation in neurodegenerative diseases such as AD and Nasu-Hakola disease. Understanding the impact of disease-associated genetic mutations in TREM2 on its function is crucial for developing targeted therapies. Earlier studies demonstrated that AD-risk variants R47H and R62H disrupt TREM2's interaction with HS, showing a role for HS in TREM2 function and AD progression. The present disclosure further verifies HS as a TREM2 ligand and reveals the formation of TREM2-HS complexes on microglial surfaces. Moreover, the functional investigations demonstrate that HS facilitates TREM2-mediated uptake of ApoE3 by microglia. These findings underscore the regulatory role of HS in TREM2 signaling and microglial functions, offering valuable insights for future research and potential therapeutic strategies.

Understanding the specific HS structures necessary for binding to TREM2 is crucial for developing targeted therapies. In the LMW HS microarray analysis, significant variation was observed in TREM2 binding among HS oligomers with similar sizes and sulfation levels. This indicates that the overall sulfation level alone does not solely determine HS binding to TREM2. Rather, specific HS structures play a critical role in this interaction. Through comparisons of TREM2-binding and non-binding HS oligomers with similar sequences, SPR competition analysis of chemically modified heparins, and examination of HS modification-deficient cells, essential HS structural requirements were identified for TREM2 binding. These include 6S and a minimum chain length of approximately 10 saccharides, with additional contributions from IdoA and possibly 3S, while NS and 2S are dispensable. This knowledge holds significant therapeutic promise, guiding the development of targeted treatments to modulate TREM2 function in neurodegenerative disease management.

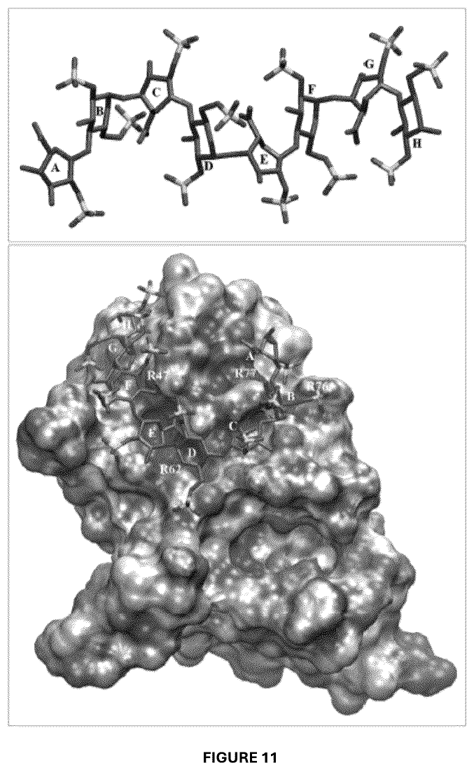

To gain deeper insights into the TREM2-HS interaction, including the specific TREM2 amino acid residues involved, the present disclosure also created an in-silico model of the interaction between a heparin octa-saccharide fragment ([IdoA2S-GlcNS6S]₄) and the A chain of TREM2. Focused docking resulted in 20 ligand poses. The top-ranked pose (FIG. 11), based on theoretical interaction energy, was used for visualization and further analysis. According to Protein-Ligand Interaction Profiler (PLIP) analysis, the heparin fragment formed hydrogen bonds and/or salt bridge interactions with seven residues of the TREM2 protein, including four arginines (R47, R62, R76, and R77), one asparagine (N68), one serine (S65), and one tryptophan (W70) (Table 2). The values of the glycosidic torsion angles were all within allowable regions of the potential energy surface. The modeling indicated that the octamer-fragment of heparin could occupy the entire putative binding site in TREM2. The experimental observation that binding to heparin oligomers was enhanced as the heparin chain length increases may arise from mass transfer (or ligand rebinding) effects due to the increase in the number of binding epitopes or may indicate an alternative binding mode.

AD is a progressive neurodegenerative disorder characterized by the accumulation of amyloid-β (Aβ) plaques, intraneuronal neurofibrillary tangles, and widespread neuroinflammation, including microglial activation. Despite extensive research, the precise molecular mechanisms underlying AD pathogenesis remain elusive. Genome-wide association studies (GWAS) and whole-exome sequencing have identified over 30 AD risk loci, including TREM2 and ApoE. TREM2, a cell surface receptor on myeloid cells, initiates multiple pathways upon ligand binding, promoting cell survival, proliferation, chemotaxis, and phagocytosis, essential for normal immune function. Notably, TREM2 is highly expressed in microglia, the resident immune cells of the central nervous system. The loss-of-function R47H mutation in TREM2 represents one of the strongest single-allele genetic risk factors for AD, implicating microglial dysfunction in AD pathogenesis. APOE, a major cholesterol carrier in circulation and the sole cholesterol transporter in the brain, has three isoforms: AD-protective ApoE2, neutral ApoE3, and high AD-risk ApoE4. TREM2 binds all three ApoE isoforms with high affinity, mediating phagocytosis of ApoE-bound apoptotic neuronal cells or Aβ by microglia or macrophages. Interestingly, the R47H mutation impairs TREM2's ability to bind both HS and ApoE. It was previously demonstrated that HS also binds ApoE, showing that HS may simultaneously interact with TREM2 and ApoE to facilitate TREM2-mediated ApoE uptake by microglia. The present disclosure supports this by demonstrating that the loss of HS disrupts TREM2-mediated ApoE uptake by microglia. A model is proposed herein in which cell surface HS acts as a coreceptor for ApoE, bridging the formation of a functional ApoE/HS/TREM2 ternary complex during microglial ApoE uptake. This process is disrupted by the AD-associated R47H mutation, contributing to AD pathogenesis. Additionally, TREM2 recognizes other ligands such as phospholipids, glycolipids, and lipoproteins like low-density lipoprotein and high-density lipoproteins, as well as Clusterin.

Several investigations have reported abnormal HS expression in the AD brain, including elevated HS levels and structural alterations evidenced by changes in protein-ligand interactions and direct biochemical analyses. Gene expression profiling has revealed dysregulation of HS biosynthesis genes, notably upregulation of Hs6st1 and Hs6st2, enzymes that add 6-O-sulfate groups to HS, and downregulation of Sulf2, which removes 6-O-sulfated groups. This dysregulation may enhance the HS-TREM2 interaction within microglia in cis or between different cell types in trans, influencing AD pathogenesis. Similarly, upregulated expression of Glce and Hs3sts in AD, enzymes responsible for converting GlcA to IdoA and adding 3-O-sulfate groups to HS, respectively, show a potential role for elevated IdoA and 3S in enhancing TREM2-HS interaction and contributing to AD pathogenesis. Notably, the level of 3-O-sulfated HS is increased in the AD brain, and 3S enhances the binding of HS to tau and ApoE. Consistent with these findings, the glycan microarray data indicate that HS binding to TREM2 may be enhanced by 3S, supporting a role for 3S in AD.

In summary, the present disclosure has elucidated the molecular intricacies of the interaction between TREM2 and HS, forming functional TREM2-HS complexes on microglia cell surfaces. This highlights the role of HS in modulating microglial functions through its interaction with TREM2. Importantly, investigations have identified the specific HS structure to which TREM2 binds, revealing crucial HS modifications necessary for this interaction. These findings carry significant implications, particularly in developing therapeutics targeting neuroinflammation. By selectively targeting the TREM2 pathway based on its interaction with specific HS structures, novel treatment avenues can be unlocked for AD and other neurodegenerative diseases. Furthermore, understanding the impact of aberrant HS expression on the TREM pathway in AD patients offer invaluable insights into disease mechanisms, paving the way for developing biomarkers and precision medicine strategies for AD.

### Experimental Procedures

Cell surface TREM2 binding assay. The biotinylated recombinant human TREM2 extracellular domain was produced and employed to assess its cell surface binding. Initially, mouse lung endothelial cell (MLEC) lines, which were generated in lab and comprised both wildtype and multiple HS mutants, were plated at a density of 4×10⁴ cells per well in a 96-well plate containing Dulbecco's Modified Eagle Medium (DMEM) supplemented with 10% fetal bovine serum (FBS), 100 U/ml penicillin, and 100 μg/ml streptomycin. After an overnight culture at 37° C. with 5% CO₂, the cells were fixed using 4% paraformaldehyde (PFA) in phosphate-buffered saline (PBS) at room temperature (RT) for 15 minutes. After washing and overnight blocking in 5% bovine serum albumin (BSA) at 4° C., the cells were incubated with biotinylated TREM2 at 0, 1, 2, or 5 g/ml for 1 hour at RT. Following another round of washing, the cells were incubated with streptavidin-HRP (1:5000 dilution) for 1 hour at RT. After thorough washing, an HRP substrate was added, and the reaction was allowed to proceed for 25 minutes before terminating with 0.5 M HCl. Absorbance at 450 nm was then measured. MLEC lines were mycoplasma negative.

Proximity Ligation Assay (PLA). C20 human microglia cells were obtained from ATCC and cultured in DMEM supplemented with 10% FBS and 1% Penicillin/Streptomycin. The cells were cultured in a controlled environment at 37° C. with 5% CO₂. The experimental procedure involved seeding C20 cells on coverslips at 40% confluency and culturing the cells accordingly. Heparinase-treated cells were utilized as the background control and were prepared by digesting the cells for 1 hour at 37° C. with heparinases I+III enzymes (R&D, Cat #7897-GH and Cat #6145-GH) at a concentration of 0.5 μg/mL (62). Subsequently, the cells were fixed with 4% PFA in PBS for 10 minutes at RT and then blocked for 1 hour using immunofluorescence blocking buffer (4% Normal Goat Serum (NGS), 1% BSA, 0.5% Triton in PBS) at 37° C. Subsequently, cells were incubated overnight at 4° C. with primary antibodies (rabbit anti-human TREM2 from Cell Signaling, Cat #91068, 1:300, and mouse anti-10E4 from AmsBio, 1:300) diluted in immunofluorescence blocking buffer. The next day, samples were washed thoroughly between steps, and incubated with 1:750 Thermo Scientific AlexaFluor secondary antibodies (anti-rabbit IgG 488 (Cat #A11008) and anti-mouse IgM 594 (Cat #A21044) for 1 hour at 37° C. After washing, samples were incubated with 1:10,000 DAPI in PBS and mounted on slides using ProLong Gold Antifade mounting medium (Thermo Fisher, Cat #P36930). Imaging was conducted using a Leica SP8 confocal microscope with a 60× objective and 3× optical zoom, capturing images at a resolution of 1024×1024. Image analysis was conducted using ImageJ/Fiji (NIH) software, and the Coloc2 plugin was utilized to determine the Pearson R correlation coefficient between TREM2 and HS immunofluorescence. C20 cells were confirmed to be mycoplasma negative.

Heparin, chemically modified heparin and heparin oligosaccharides. The porcine intestinal heparin (16 kDa) was from Celsus Laboratories, Cincinnati, OH. N-desulfated heparin (N-Des Hep, 14 kDa) and 2-O-desulfated heparin (2-Des Hep, 13 kDa) were prepared. Additionally, 6-O-desulfated heparin (6-Des Hep, 13 kDa) was prepared. These chemically modified heparins were confirmed to have no anticoagulant activity via the amidolytic anti-factor Xa assay and were negative for endotoxin using the Limulus test. Heparin oligosaccharides, including tetrasaccharide (dp4, Cat #H004), octasaccharide (dp8, Cat #H008), decasaccharide (dp10, Cat #H010), and hexadecasaccharide (dp16, Cat #H016), were from Iduron (Manchester, UK). These oligosaccharides were prepared by controlled partial depolymerization of unfractionated heparin by heparinase I followed by separation via high-resolution gel filtration.

Assessing the interaction between TREM2 and Heparin using Surface Plasmon Resonance Analysis. The surface plasmon resonance (SPR) analysis was conducted using a BIAcore 3000 instrument (Cytiva, Uppsala, Sweden). The heparin chip utilized had been prepared in prior investigations by immobilizing biotinylated heparin on a sensor streptavidin SA chip from Cytiva (Uppsala, Sweden). For the direct binding analysis, TREM2 (Sino Biological, Cat. #11804-H08H) was appropriately diluted in a running buffer composed of 0.01 M HEPES, 0.15 M NaCl, 3 mM EDTA, and 0.005% surfactant P20, adjusted to pH 7.4 (referred to as HBS-EP buffer). TREM2 proteins at various concentrations was then injected over the heparin chip at a constant flow rate of 30 μl/min. After the injection phase, a buffer was passed over the sensor surface to facilitate dissociation. Following a 3-minute interval, the sensor surface was regenerated by injecting 30 μl of 2 M NaCl solution. The response was continuously monitored over time (as a sensorgram) at 25° C. To extract kinetic parameters such as the association rate constant (kₐ), dissociation rate constant (k_{d}), and equilibrium dissociation constant (K_{D})), the sensorgrams obtained from injections of various concentrations of TREM2 were globally fitted. This fitting procedure was performed using the BiaEvaluation software version 4.0.1 (Cytiva, Uppsala, Sweden), assuming a 1:1 Langmuir model. The experiments were repeated three times. In the solution competitive binding analysis, the SPR experiment followed a similar protocol. wTREM2 at a concentration of 170 nM was pre-mixed with 1000 nM of chemically modified heparins. Alternatively, TREM2 at a concentration of 250 nM was pre-mixed with 1000 nM of heparin oligos, including dp4, dp8, dp10, and dp16, all in HBS-EP buffer.

Low molecular weight HS microarray. A microarray chip was prepared by immobilizing ninety-six structurally defined low molecular weight (LMW) HS mimetic oligosaccharides at a concentration of 25 μM using established procedures. Each compound was printed with 36 spots in a 6×6 square. The chip was then incubated with 100 μl His-tagged human TREM2 at a concentration of 10 μg/ml in PBS for 1 hour at RT. Concurrently, 100 μl of 1 μM HIS Lite™ OG488-Tris NTA-Ni complex was also applied to the chip. Post-incubation, non-specifically bound fluorophores and proteins were washed off from the chip through two wash cycles. Fluorescence was excited at 488 nm using a GenePix 4,300 scanner (Molecular Dynamics, Caesarea, Israel). The scanner's resolution was set at 5 μm precision. The resulting array images were analyzed using GenePix Pro 7.2.29.002 software to obtain mean fluorescence intensities via the software's array quality control feature. Automated detection localizes the printed spots; manual correction rectifies any discrepancies observed during this process. Finally, mean fluorescence intensities for each oligosaccharide spot were plotted against their respective identities employing GraphPad Prism version 9.3.1.

RNA purification and quantitative RT-PCR. Total cellular RNA was extracted from C20 cells using TRIzol reagent (Invitrogen). cDNA was synthesized using iScript cDNA synthesis kit (Cat #1708891, Bio-rad, USA), according to the manufacturer's instructions. Quantitative RT-PCR was carried out in 20-μl volumes that included 10 μl of iTag Universal SYBR Green Supermix (Cat #1725124, Bio-rad), 5 μl of diluted RT products as template, and 20 pmol of individual primer. Primer sequences used included the following: 5-ATGATGCGGGTCTCTACCAGTG (SEQ ID NO: 1)-3 (forward) and 5-GCATCCTCGAAGCTCTCAGACT (SEQ ID NO: 2)-3 (reverse) for human Trem2; 5-GCTCTTGTCTCGCCCTTTTGT (SEQ ID NO: 3)-3 (forward) and 5-GTGGTGCAAGCCATTCCTAC (SEQ ID NO: 4)-3 (reverse) for human Ext1; 5-ATGTGTGCGTCGGTCAAGTAT (SEQ ID NO: 5)-3 (forward) and 5-AGAATGGGGCCAAAACTGAAA (SEQ ID NO: 6)-3 (reverse) for human Ext2; 5-ACGCCCAGGAAGTTCTACTAC (SEQ ID NO: 7)-3 (forward) and 5-GTTGTACGGGCAGTCCATGAA (SEQ ID NO: 8)-3 (reverse) for human Hs6st1; 5-CATGGGGCCGAAAATCTTGGA (SEQ ID NO: 9)-3 (reverse) for human Hs6st2; 5-TCCAGTGTCACGTTACCTGAG (SEQ ID NO: 10)-3 (forward) and 5-TGTAGGTGCAATCCATAAACTCC (SEQ ID NO: 11)-3 (reverse) for human Hs6st3; 5-GTCTCCTCTGACTTCAACAGCG (SEQ ID NO: 12)-3 (forward) and 5-ACCACCCTGTTGCTGTAGCCAA (SEQ ID NO: 13)-3 (reverse) for human GAPDH. All primers used in qRT-PCR were synthesized by Integrated DNA Technologies (IDT, USA). The PCR amplifications (40 cycles at 95° C. for 5 s and at 60° C. for 30 s) were performed using the Bio-Rad CFX96 real-time system. For each target gene, the average Ct values were calculated from six replicates, which were then normalized to the average Ct values for GAPDH. These normalized values were used to calculate a value expressing the extent of knockdown relative to the nonspecific control siRNA, based on the 2^{-ΔΔCT} method.

RNA interference (RNAi) transfection. C20 cells (2×10⁵/well) were plated in 6-well plates. Next day, RNAi transfection was conducted using Dicer substrate siRNAs (DsiRNAs) purchased from Integrated DNA technologies or On-TARGET plus SMARTpool human Hs6st3 (Dhamscon, USA) and the Lipofectamine RNAiMAX transfection reagent (REF-13778; Invitrogen, USA) following the manufacturer's instructions, including NC-1 (IDT), a negative control DsiRNA with no homology to any human gene as a control. DsiRNA sequences used included the following: 5-GUCCUGAGUCUGGAUACUUUAGACA (SEQ ID NO: 14)-3 for human Ext1; 5-CUACGAGAAGAAGUACUACUUCCCG (SEQ ID NO: 15)-3 for human Hs6st1; 5-AACACCUGACAACUUCUGAAUAUUG (SEQ ID NO: 16)-3 for human Trem2. Forty-eight hours after infection, the cells were harvested for RNA extraction or subjected to function study. The transfection efficiency was monitored via qRT-PCR.

ApoE internalization assay. The expression, purification, and conjugation of human ApoE3 with Alexa Fluor 488 (ApoE3-488). C20 cells, including those transfected with either an empty vector or siRNA targeting TREM2, Ext1, Hs6st1, or Hs6st3, were seeded at a density of 3×10⁴ cells per well in 300 μL of DMEM supplemented with 10% FBS, 100 U/mL penicillin, and 100 μg/mL streptomycin. After overnight culture, the cells were washed twice with DPBS and then incubated with 300 μL/well of DMEM containing ApoE3-488 (3 μg/mL) at 37° C. for 8 hours. Subsequently, the cells underwent image analysis. They were covered with mounting medium containing DAPI and examined for internalized ApoE3-488 using a Keyence BZ-Z800 microscope. Image data were processed using ImageJ.

Statistical Analysis. Statistical analysis was carried out with Prism 8 for Macintosh. All data are presented as mean±SD or mean±SEM and analyzed using a student's t-test for two-group comparison. p values less than 0.05 were chosen as a threshold for statistical significance.

### Example 2: Microglial Heparan Sulfate Interact with TREM2 Suppressing TREM2 Signaling to Block Amyloid-β in the Brain, and this Pathway can be Targeted for Treatment

Alzheimer's disease (AD) is a progressive neurodegenerative disorder marked by amyloid-β (Aβ) deposition, hyperphosphorylated tau accumulation, and widespread neuroinflammation. No current treatments can cure AD or halt the disease progression, making it critical to discover new mechanisms that drive the disease. Mounting evidence suggests that Aβ accumulation is an early and pivotal event in AD. Kinetic studies indicate that impaired Aβ clearance, rather than overproduction, is the primary cause of Aβ accumulation in sporadic AD. However, the molecular mechanisms governing Aβ clearance remain poorly understood, representing a significant gap in AD research. It has been revealed that expression of heparan sulfate (HS), a sulfated polysaccharide crucial for cell-matrix and cell-cell interactions and signaling, is significantly downregulated in microglia of AD patients. Replicating the reduced microglial HS expression ameliorates Aβ accumulation in an AD mouse model. Herein, it is contemplated that microglial HS impedes Aβ clearance in AD pathogenesis by suppressing the Triggering Receptor Expressed on Myeloid cells 2 (TREM2) pathway, and reducing microglial HS is a therapeutic target for AD treatment. The present disclosure demonstrates the following: 1) Delineate the roles of microglial HS in Aβ pathology in AD; 2) Elucidate the roles and mechanisms of microglial HS in regulating Aβ clearance in AD; 3) Determine the TREM2-binding HS structure and target microglial HS for AD treatment. The present disclosure also employs novel and established genetic, cellular, single-cell RNA sequencing, ATAC-seq, ChIP-seq, proteomics, and biochemical approaches, alongside the examination of human specimens. The present disclosure also delineates the detrimental role of microglial HS in AD, illuminates the underlying molecular mechanisms and related HS structures, and establishes the concept that targeting microglial HS is an effective intervention to mitigate AD pathogenesis.

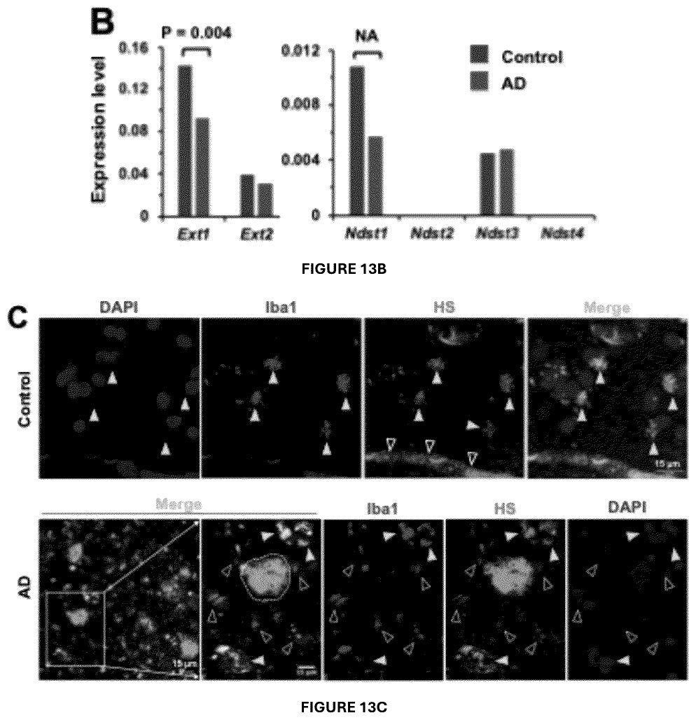

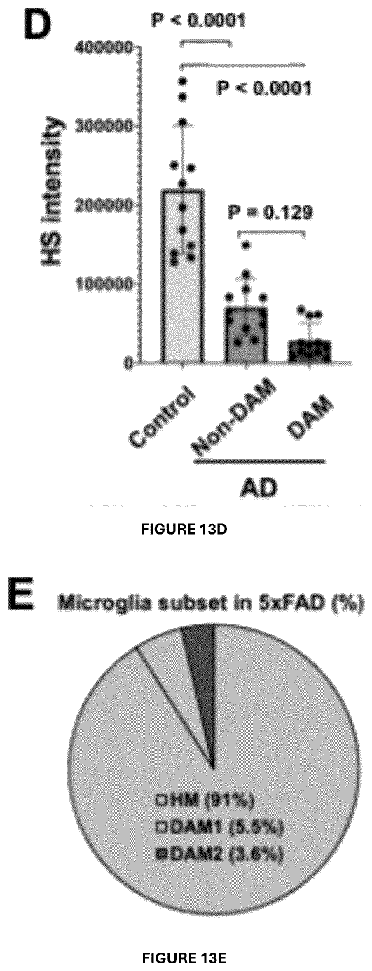

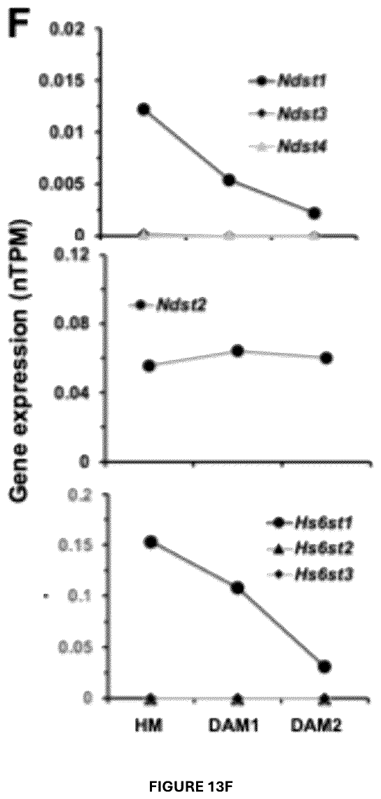

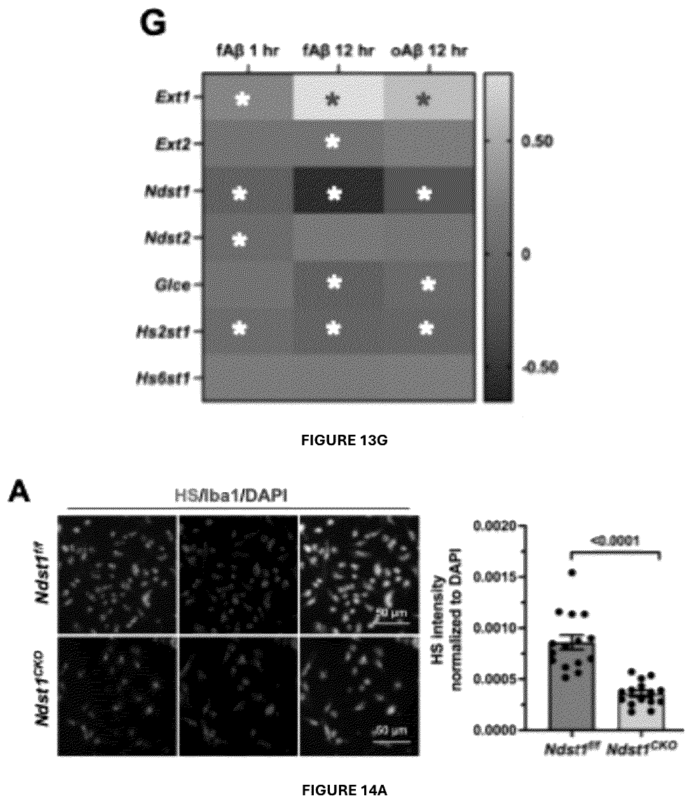

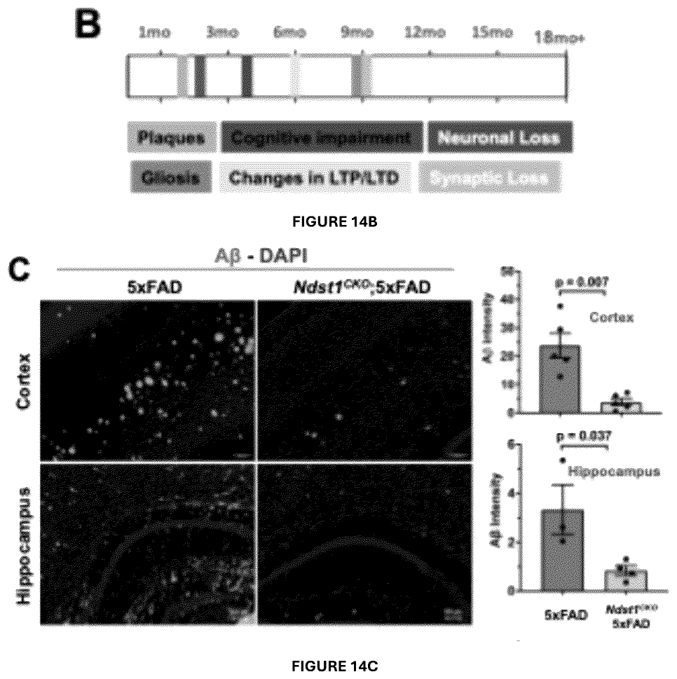

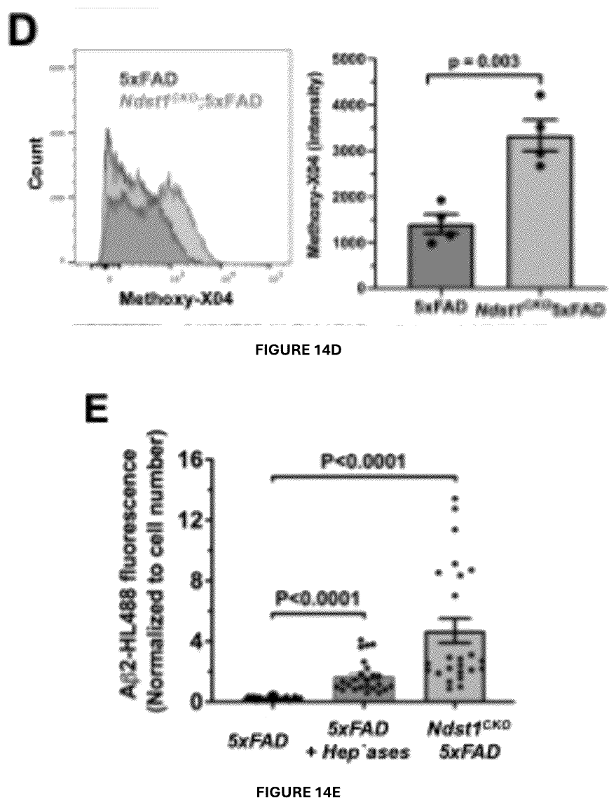

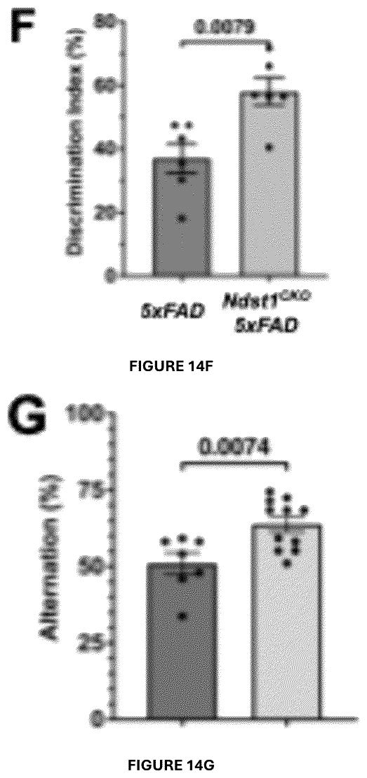

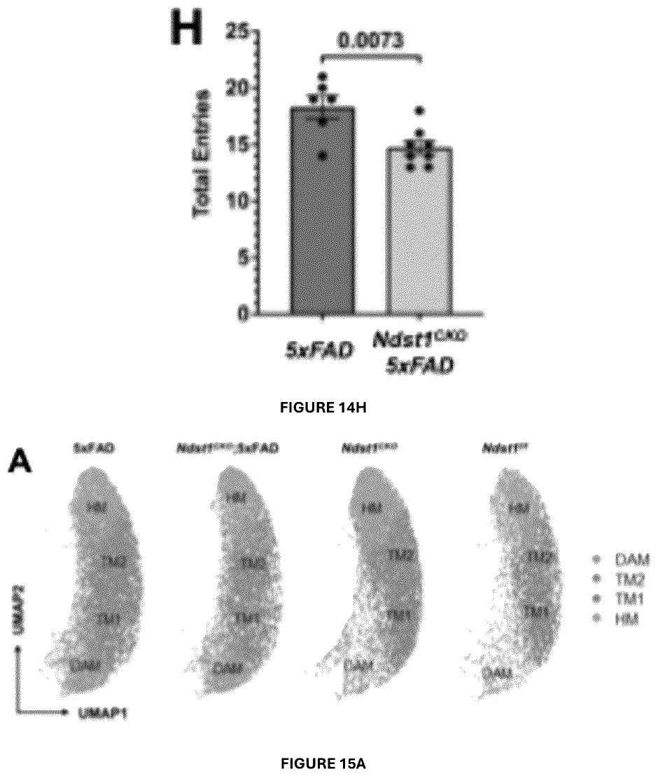

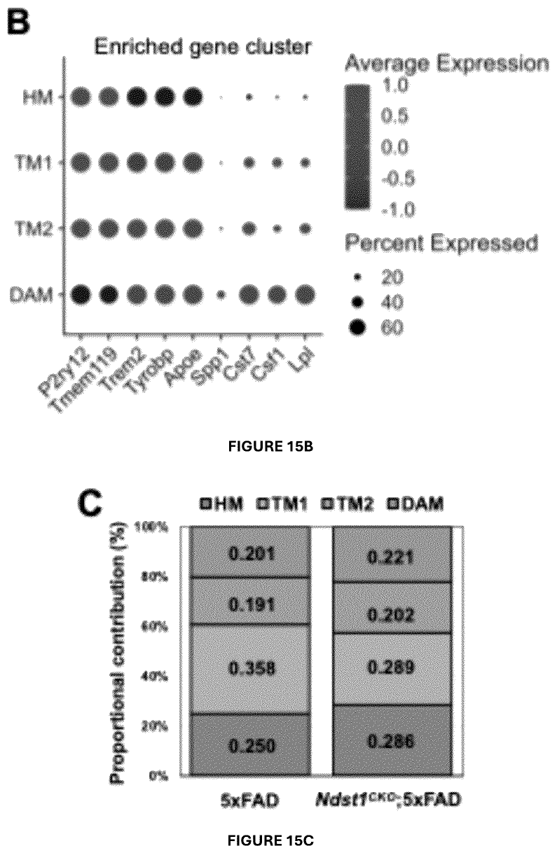

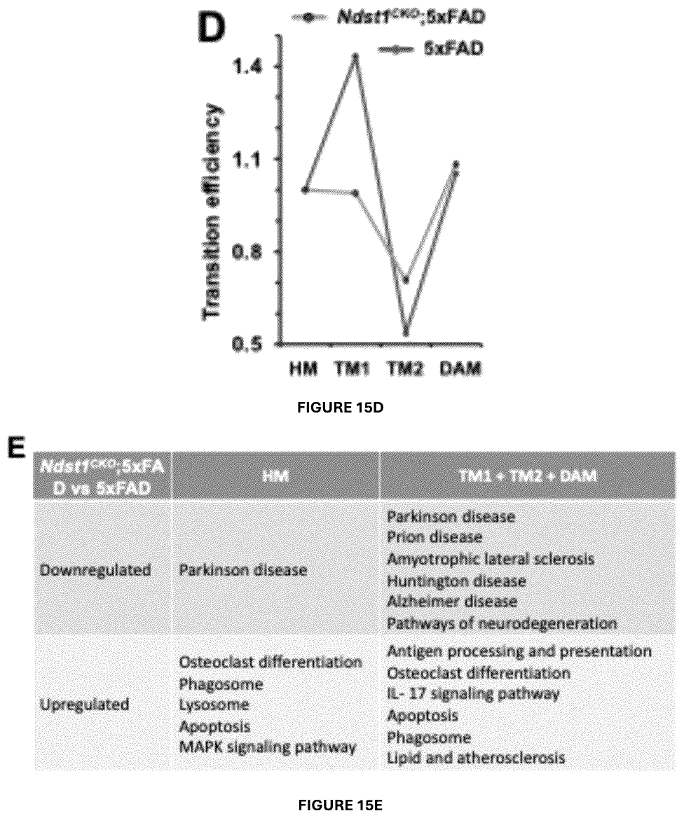

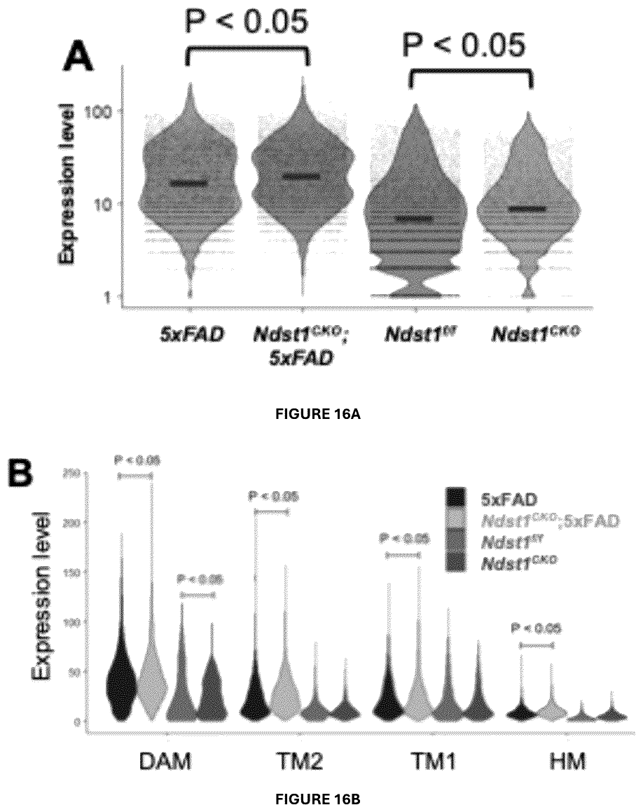

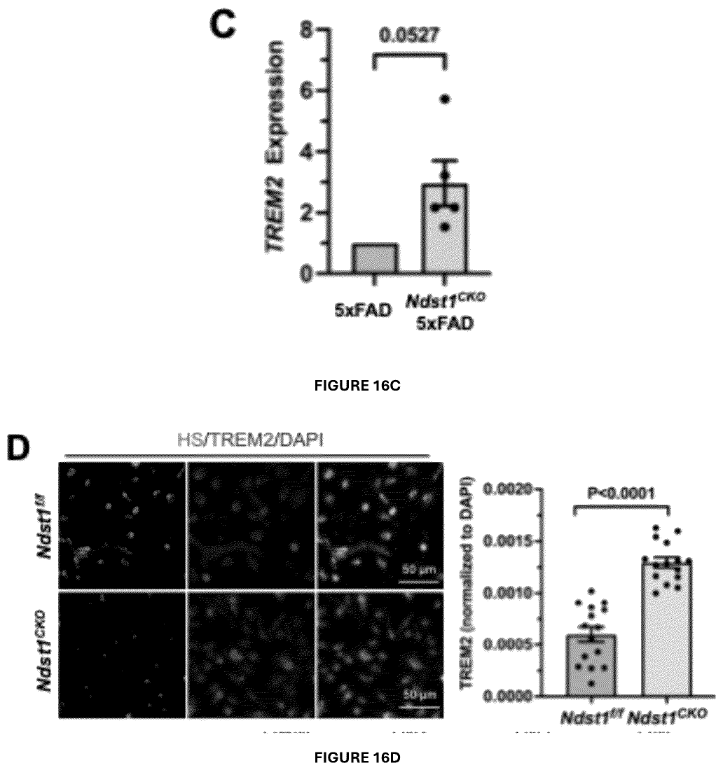

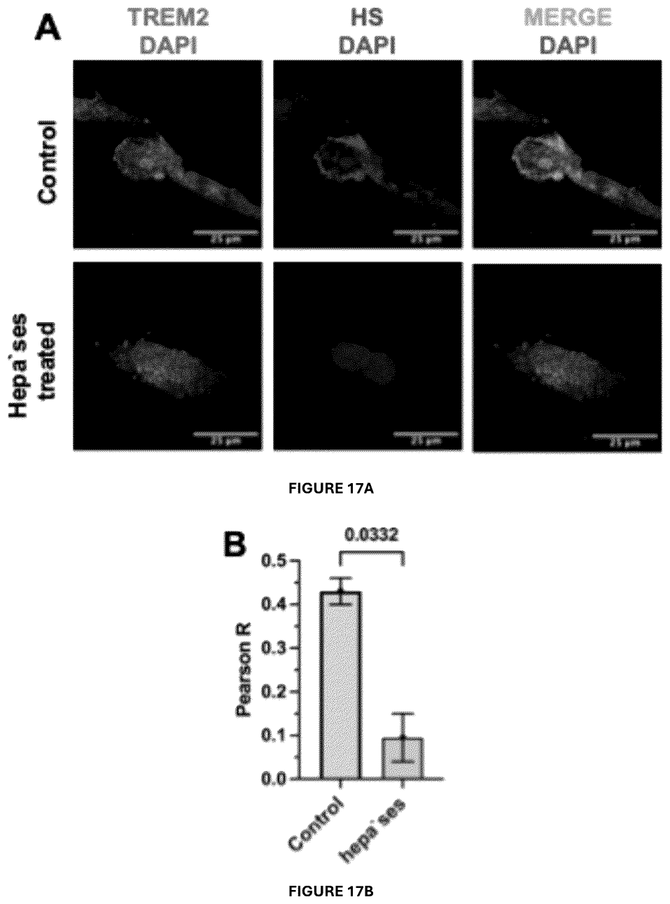

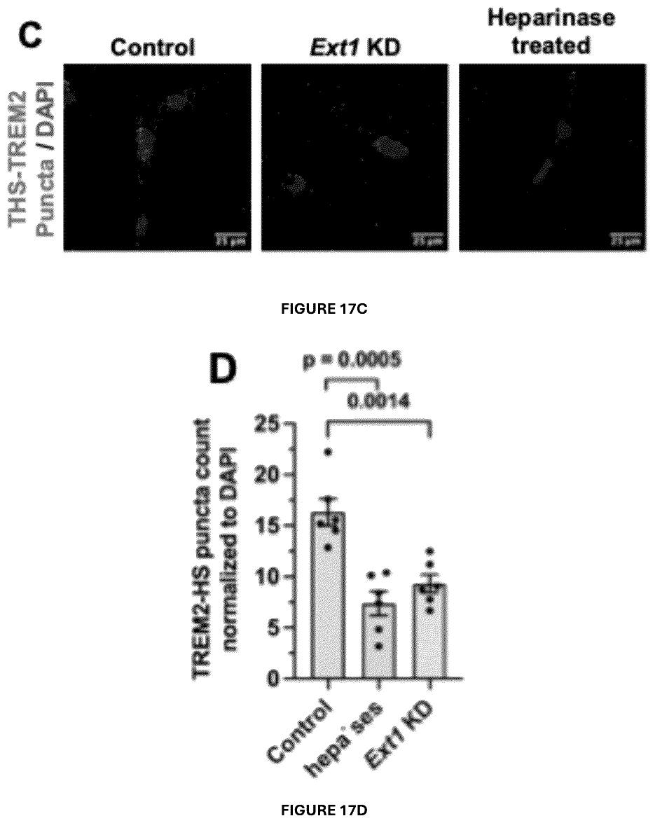

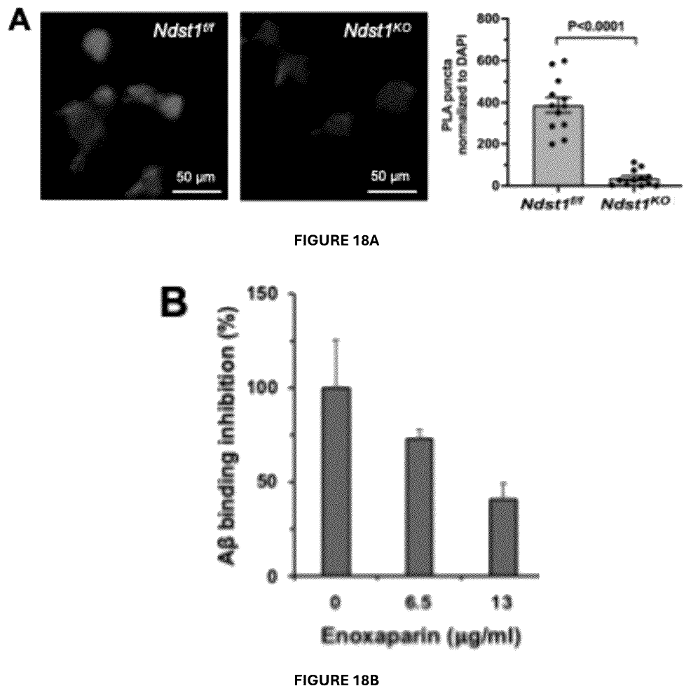

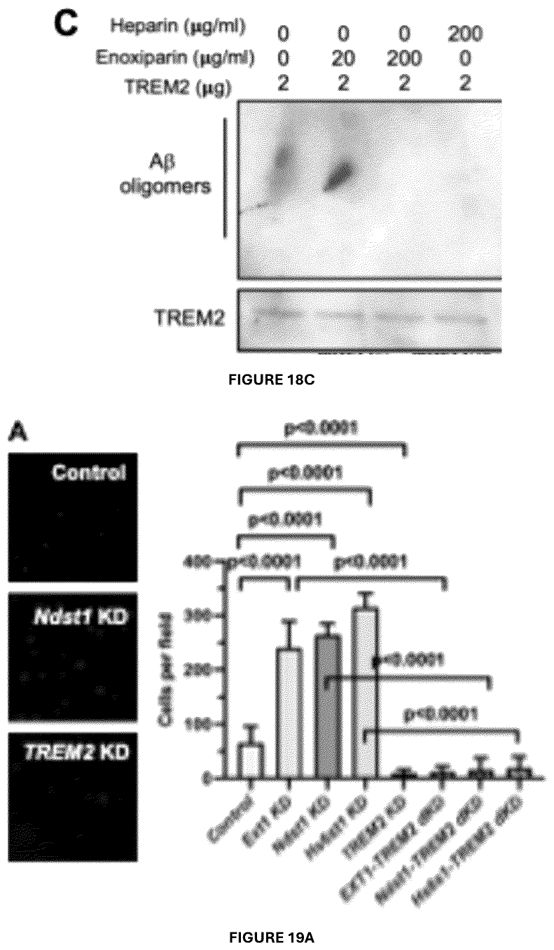

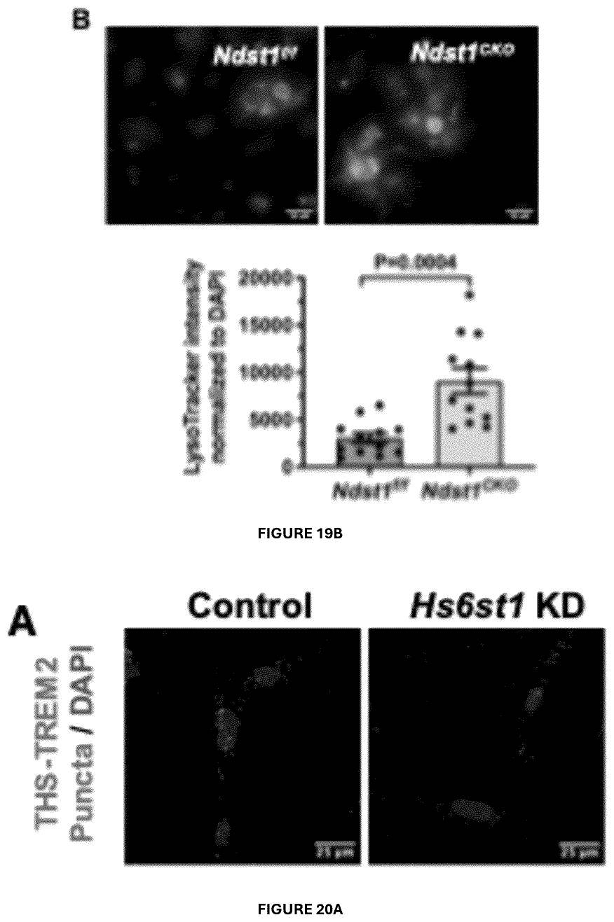

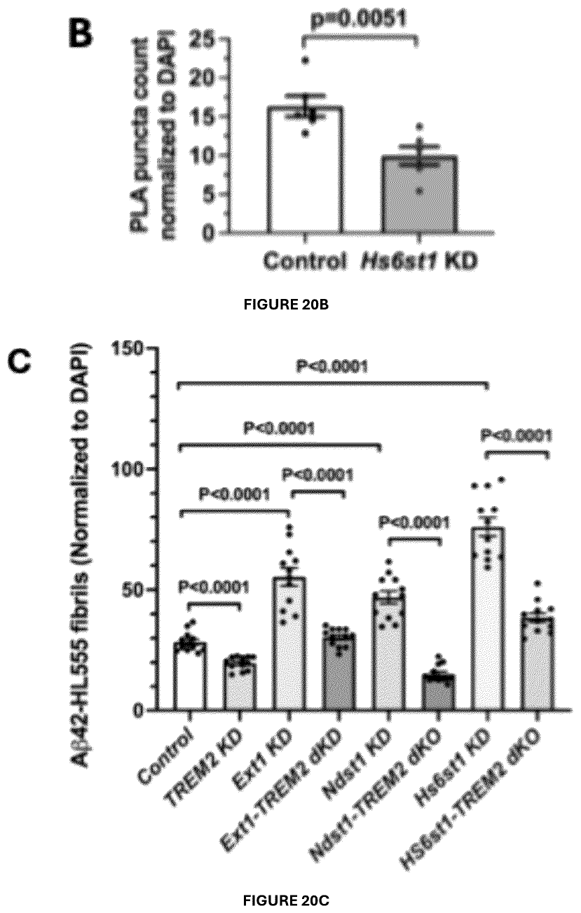

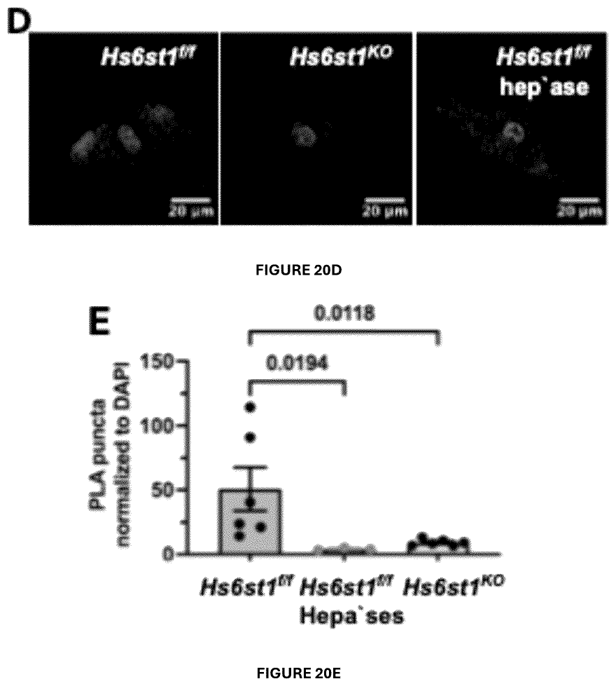

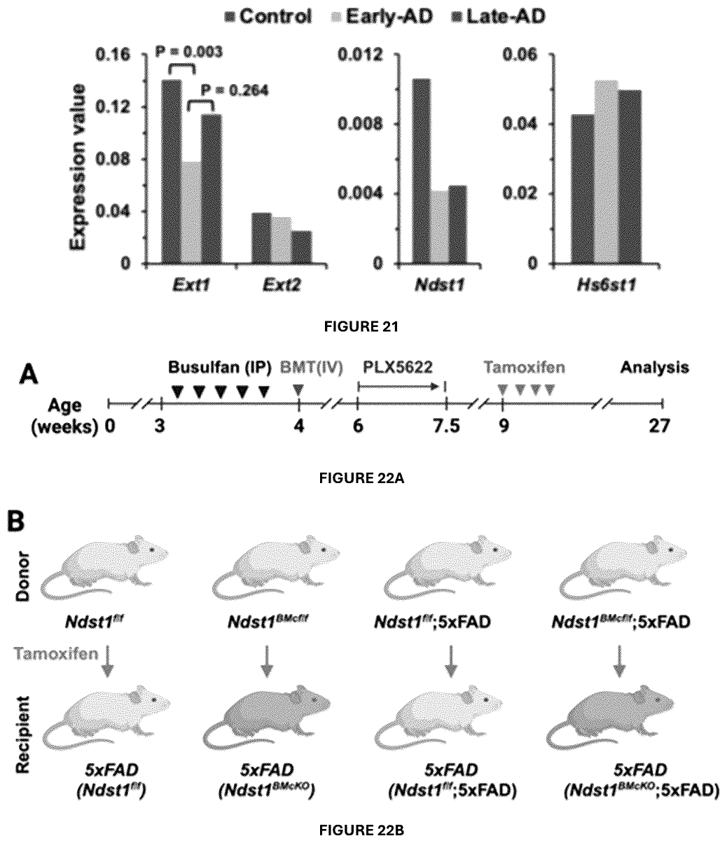

To understand AD pathogenesis, the present disclosure has revealed: 1) HS and its biosynthesis genes Ext1 and Ndst1 are downregulated in microglia in AD patients, although their expression remains high across early and late AD stages (FIGS. 13B, 13C, 13D, and FIG. 21); 2) Ndst1 and HS biosynthesis gene Hs6st1 are also downregulated in disease-associated microglia (DAM) in 5×FAD mice (FIGS. 13E and 13F) and Aβ-treated microglia (FIG. 13G); 3) Microglial Ndst1 knockout (Ndst1^{CKO}) reduces Aβ accumulation, enhances microglial Aβ phagocytosis and improves cognition in 5×FAD mice (FIGS. 14A, 14B, 14C, 14D, 14E, 14F, 14G, and 14H); 4) Ndst1 knockout increases DAM population, downregulates AD-related pathways, and upregulates phagocytosis-related pathways in 5×FAD microglia (FIGS. 15A, 15B, 15C, 15D, and 15E); 5) Ndst1 knockout upregulates TREM2 expression in microglia (FIGS. 16A, 16B, 16C, and 16D); 6) HS binds TREM2, forming HS-TREM2 complexes on the microglial surface (FIGS. 17A and 17B); 7) Ndst1 deletion disrupts HS-TREM2 complex formation in bone marrow-derived macrophages (BMDM) and microglia, and heparin prebinding blocks TREM2-Aβ interaction (FIGS. 18A, 18B, and 18C); 8) HS requires 6-O-sulfation to bind TREM2, and Hs6st1 deletion reduces HS-TREM2 complexes in microglia and BMDMs, enhancing microglial migration and Aβ phagocytosis (FIG. 19A and FIGS. 20A, 20B, 20C, 20D, and FIGS. 2D, 2E, 3A, 3B, 4B, 4C, 5A, 5B, 5C, 5D, 5E, 5F, 6C, and 6D). The protective effects of microglial Ndst1 deletion resemble those of microglial TREM2 overexpression or activation in 5×FAD mice. Thus, the present disclosure contemplates that microglial HS impedes Aβ clearance in AD by forming HS-TREM2 complexes and downregulating TREM2 expression, thereby suppressing TREM2-mediated Aβ clearance. Reducing microglial HS, particularly 6-O-sulfation, is a promising target for AD treatment (FIGS. 2D, 2E, 4B, 4C, 6C, 6D, 12A, 12B, 3A, 3B, 5A, 5B, 5C, 5D, 5E, 5F, 19A, 19B, 20A, 20B, 20C, 20D, and 20E).” Herein, the following aspects are examined:

Delineation of the roles of microglial HS in Aβ pathology in AD. The the spatial expression of microglial HS, HS genes, and DAM markers are analyzed in human AD using immunostaining and the NanoString GeoMx DSP platform. In 5×FAD mice, it is assessed how microglial Ndst1 deficiency affects Aβ metabolism, tau phosphorylation, neuroinflammation, neurodegeneration, and cognition. Microglial Ndst1 knockout are induced after A deposition to evaluate its impact during mid-to-late stages of pathology.

Elucidation of the roles and mechanisms of HS in regulating microglia functions in AD. The scRNA-seq findings are validated at mRNA and protein levels, and it is determined how Ndst1 deletion alters microglial subclusters, cytokine expression, migration, clustering, Aβ phagocytosis and degradation, and TREM2 shedding. A reporter assay confirms whether Ndst1 deletion upregulates TREM2 expression. ATAC-seq and ChIP-seq is used to define the underlying regulatory programs. Additionally, it is assessed how Ndst1 deletion disrupts Aβ-TREM2 complex formation, downstream signaling, and related microglial functions. Finally, microglial TREM2 knock-out is used to test whether the protective effects of Ndst1 deletion in 5×FAD mice are TREM2-dependent.

Determination the TREM2-binding HS structure and target microglial HS for AD treatment. The HS structure has been found that binds TREM2. It is further determined whether Hs6st1 deletion produces protective effects similar to Ndst1 deletion in 5×FAD mice. Microglia in 5×FAD mice are replaced with Ndst1- or Hs6st1-deficient BMDMs to assess therapeutic potential and evaluate their impact on Aβ pathology.

In summary, present disclosure elucidates a detrimental role of microglial HS in AD, elucidates the modulating effect of the TREM2 pathway as the underlying molecular mechanism, and establishes the concept that reducing microglial HS, particularly 6-O-sulfation, is an intervention to mitigate AD pathogenesis. These findings significantly advance the understanding of AD and open new avenues for therapy.

AD is a progressive neurodegenerative disorder marked by Aβ deposition, hyperphosphorylated tau accumulation, and widespread neuroinflammation. Despite extensive research, no treatments currently exist that cure AD or halt its progression. Identifying new mechanisms underlying AD development is crucial for developing effective therapies. Mounting evidence shows that Aβ accumulation is an early and pivotal event in AD. Notably, kinetic studies indicate impaired Aβ clearance, rather than overproduction, is the leading cause of Aβ buildup in sporadic AD cases. However, the cellular and molecular mechanisms governing Aβ clearance remain poorly understood, representing a significant gap in AD research and hindering progress toward effective disease-modifying treatments.

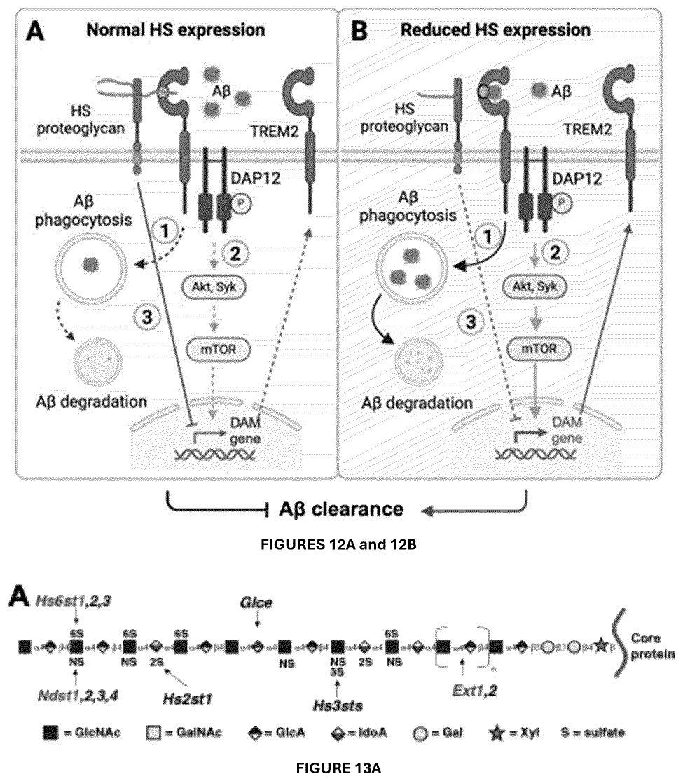

Microglia, TREM2, and disease-associated microglia. Recent studies emphasize the critical role of microglia and Triggering Receptor Expressed on Myeloid cells 2 (TREM2) in AD pathogenesis, particularly in Aβ clearance (FIG. 12A). Microglia, the brain's resident immune cells, are key responders to Aβ plaques. TREM2, an Aβ receptor expressed on microglia, is a major genetic risk factor for AD. In the 5×FAD transgenic amyloid mouse model, TREM2 facilitates the transition from homeostatic microglia (HM) to disease-associated microglia (DAM), which cluster around Aβ plaques to form a protective barrier that limits plaque growth and neuronal damage. TREM2 deficiency impairs this response, resulting in reduced phagocytic activity and increased plaque burden and neurodegeneration. Conversely, TREM2 overexpression reprograms microglia toward a more phagocytic, less inflammatory phenotype, enhancing Aβ clearance and mitigating pathology in an AD mouse model. Additionally, TREM2-activating antibodies improve microglial function, promoting Aβ clearance, and attenuate AD pathology in mouse models. These findings underscore the therapeutic potential of targeting the TREM2 pathway and highlight the critical importance of discovering new mechanisms that control TREM2 activation to develop effective AD treatments.

Critical and complex roles of heparan sulfate in Aβ clearance. Heparan sulfate (HS) is a linear polysaccharide with four types of sulfation modifications, namely N-sulfation (NS) and 2-, 3-, and 6-O-sulfation (2S, 3S, and 6S) (FIG. 13A), which create specific ligand-binding sites and confer HS to interact with a wide range of protein ligands to regulate various cellular signaling and functions. HS biosynthesis involves over 20 enzymes responsible for chain elongation and sulfation (FIG. 13A), with expression varying by cell type, tissue, and physiological or pathological context. During synthesis, HS is covalently linked to core proteins to form HS proteoglycans (HSPGs), expressed on the cell surface and in the extracellular matrix. HS co-localizes with Aβ in plaques in AD patients and mouse models, promotes Aβ aggregation, and mediates Aβ internalization and cytotoxicity in biochemical and cell studies. In vivo, neuronal HS has been shown to trap Aβ, impeding its clearance in mouse models, while the present disclosure indicates that endothelial HS facilitates Aβ clearance in 5×FAD mice. Additionally, human population genetic studies have identified two AD-protective variants—APOE3 Christchurch (ApoE3 R136S), which has reduced HS affinity, and RELN COLBOS, which has increased HS affinity, both of which confer protection in AD mouse models. These findings underscore the intricate, cell-type- and molecular pathway-specific roles of HS in AD and may also explain the conflicting treatment outcomes of heparin, a highly sulfated HS analog, in AD models. A deeper understanding of HS functions across brain cell types and related molecular pathways is essential for advancing AD research and developing targeted, effective therapies.

Microglial HS is downregulated in AD patients, and considered to be even lower in DAM. In human AD brains, overall HS levels are elevated; however, whether this reflects increased biosynthesis remains unclear. To investigate, single-cell RNA sequencing (scRNA-seq) data is analyzed from 24 AD patient forebrains and 24 matched controls (GEO/synapse number: syn18485175). Among the HS biosynthesis genes, Ext1 is significantly downregulated in microglia, while Ndst1 showed a strong trend toward downregulation (FIG. 13B). HS gene expression remained unchanged in other major brain cell types, including neurons, oligodendrocytes, and astrocytes. Ext1 and Ext2 form a functional heterodimer essential for HS chain polymerization and determine the overall level of HS biosynthesis. The reduced microglial Ext1 and Ndst1 expression, along with no changes in other cells, shows that the elevated HS levels in AD are more likely due to impaired degradation rather than increased biosynthesis. This is supported by evidence that HS co-deposits in Aβ plaques and Aβ binding inhibits HS degradation by heparanase. Ndst1 initiates HS modification reactions by adding NS, the most common modification. Downregulation of Ext1 and Ndst1 is decreases the length of HS chains and the abundance of NS. The staining of anti-HS antibody 10E4, which binds NS-containing HS epitopes, reflects both HS abundance and NS modification. Co-staining for microglia (Iba1+) and HS (10E4) revealed significantly lower HS levels in microglia from AD patient forebrains (FIGS. 13C and 13D), consistent with reduced expression of Ext1 and Ndst1. Notably, microglia associated with Aβ plaques, the DAM, showed a trend toward even lower HS expression than non-DAM. Neuronal HS staining remained unchanged, aligning with its stable HS gene expression. These findings reveal reduced microglial HS expression in AD, and is considered to be even lower in DAM.

Microglial HS gene expression is reduced in DAM in 5×FAD mice and downregulated by Aβ. In 5×FAD mice, microglia are categorized into HM, stage-1 DAM (DAM1), and DAM2. At six months of age, the microglial population comprises 91% HM, 5.5% DAM1, and 3.6% DAM2 (FIG. 13E). scRNA-seq data revealed that Ndst1 and another HS biosynthesis gene, Hs6st1, are progressively downregulated as microglia transition from HM to DAM1 and DAM2 (FIG. 13F). Given that DAM development in 5×FAD mice is driven by Aβ, it is contemplated whether Aβ directly induces HS gene downregulation in microglia. Bulk RNA-seq of primary mouse microglia treated with Aβ fibrils and oligomers (oAβ) showed that while Ext1 was upregulated, several HS genes, including Ext2, Ndst1, Ndst2, Glce, Hs2st1, and Hs6st1, were downregulated (FIG. 13G). These changes reduce Ext1-Ext2 heterodimer formation, reducing HS chain elongation and sulfation, and ultimately lowering HS expression. This pattern mirrors findings in human AD and DAM in 5×FAD mice. Together, these results demonstrate that Aβ exposure downregulates HS expression in microglia and show that reduced HS expression may contribute to DAM development and function, thereby influencing AD pathogenesis.

The present disclosure has also uncovered: 1) Microglial Ndst1 knockout (Ndst1^{CKO}) reduces Aβ accumulation, enhances microglial Aβ phagocytosis and improves cognition in 5×FAD mice (FIGS. 14A, 14B, 14C, 14D, 14E, 14F, 14G, and 14H); 2) Ndst1 knockout increases DAM population, downregulates AD-related pathways, and upregulates phagocytosis-related pathways in 5×FAD microglia (FIGS. 15A, 15B, 15C, 15D, and 15E); 3) Ndst1 knockout upregulates TREM2 expression in microglia (FIGS. 16A, 16B, 16C, and 16D); 4) HS binds TREM2, forming HS-TREM2 complexes on the microglial surface (FIGS. 17A and 17B); 5) Ndst1 deletion disrupts HS-TREM2 complex formation in bone marrow-derived macrophages (BMDM) and microglia, and heparin prebinding blocks TREM2-Aβ interaction (FIGS. 18A, 18B, and 18C); 6) HS requires 6S to bind TREM2, and Hs6st1 deletion reduces HS-TREM2 complexes in microglia and BMDMs, enhancing microglial migration and Aβ phagocytosis (FIGS. 19A and 20). The protective effects of microglial Ndst1 deletion resemble those of microglial TREM2 overexpression or activation in 5×FAD mice. Based on these findings, it is contemplated that microglial HS impedes Aβ clearance in AD by forming HS-TREM2 complexes and downregulating TREM2 expression, thereby suppressing TREM2-mediated Aβ clearance. Reducing microglial HS, particularly 6-O-sulfation, is a promising target for AD treatment (FIGS. 2D, 2E, 3A, 3B, 4B, 4C, 5A, 5B, 5C, 5D, 5E, 5F, 6C, 6D, 12A, 12B, 19A, 20A, 20B, 20C, 20D, and 20E).

Chronic neuroinflammation has emerged as a central theme in neurodegeneration research, particularly in AD, where microglia are increasingly recognized as key mediators of disease progression and promising therapeutic targets. The present disclosure poised to make significant contributions to the field by: 1) demonstrating that reduced expression of HS in microglia, observed in AD patients, plays a protective role in AD pathogenesis and revealing a molecular mechanism underlying the beneficial functions of microglia in AD; 2) showing that reduced HS expression promotes the generation of protective DAM; 3) elucidating reduced microglial HS upregulates TREM2 expression and enhances TREM2-Aβ interaction to promote Aβ clearance; 4) defining the necessity of 6S and Hs6st1 in regulating TREM2 pathway; and 5) establishing the concept of targeting microglial HS, particularly 6S, as an novel, selective, and effective strategy for AD treatment. These findings are highly significant, offering critical insights into the molecular mechanisms of protective neuroinflammation in AD and opening new avenues for translational applications. Ultimately, this work informs the development of innovative, mechanism-based therapies to mitigate AD disease progression.

The present disclosure introduces multiple innovative concepts and approaches. Conceptually, it is highly novel in: 1) Demonstrating that microglial HS plays a detrimental role in AD pathogenesis; 2) Identifying reduced microglial HS expression as a previously unrecognized molecular mechanism underlying beneficial microglial functions in AD; 3) Showing that microglial HS suppresses brain Aβ clearance, thereby promoting AD progression; 4) Revealing that HS inhibits DAM generation, downregulates TREM2 expression, and forms HS-TREM2 complexes that block TREM2-mediated Aβ clearance, collectively driving HS's detrimental effects in AD (FIG. 12A); 5) Introducing the concept of reducing microglial HS as a novel therapeutic strategy for AD (FIG. 12B); and 6) Determining that 6S is required for HS-TREM2 binding, identifying Hs6st1 as the key 6S-modifying gene in microglia, and establishing Hs6st1 as a selective target to enhance TREM2 signaling for AD therapy. Technically, the present disclosure is highly innovative through: 1) The use of inducible, microglia-specific HS-deficient mice in combination with the 5×FAD AD model; 2) A novel, high-efficiency microglia replacement strategy to reduce brain-resident BDMD HS in adult mice; 3) Comprehensive, unbiased multi-omics approaches including scRNA-seq, NanoString GeoMx spatial profiling, ATAC-seq, ChIP-seq, and proteomics; and 4) An integrated experimental design spanning human specimens, modified HS analogs, and genetically modified cells and mouse models to dissect the underlying cellular and molecular mechanisms and related HS structure.

As mentioned above, the present disclosure delineates the roles of microglial HS in Aβ pathology in AD and shows that replicating reduced microglia HS expression ameliorates Aβ deposition and enhances Aβ phagocytosis in 5×FAD mice. In adult mice, Ndst1 and Ndst2 are the primary Ndst genes expressed, acting downstream of Ext1 in HS biosynthesis. Double knockout (KO) of Ndst1 Ndst2 or Ext1 KO yields similar phenotypes, indicating functional overlap. Ndst1 KO mice show more severe phenotypes than Ndst2 KO, showing a dominant role for Ndst1. As Ndst1 is highly expressed in microglia in humans (FIG. 13B) and 5×FAD mice (FIG. 13F), its deletion may best model the reduced microglial HS expression in AD. We generated microglia-specific Ndst1 knockout (Ndst1^{CKO}) mice by crossing Ndst1^{f/f} mice with TMEM119-CreERT2 mice, followed by tamoxifen (TAM) induction. tdTomato A14 reporter and Iba1 staining confirmed highly efficient, microglia-specific Cre activity. Primary Ndst1^{CKO} microglia showed reduced surface HS (FIG. 14A), resembling HS loss in AD microglia. Ndst1^{CKO} mice appeared normal up to 24 months, with no changes in weight, behavior, lifespan, or microglial number/morphology, indicating Ndst1 is dispensable for microglial homeostasis. To assess effects on AD pathology, Ndst1^{CKO}; 5×FAD mice are generated with Ndst1 deletion induced at one month, prior to Aβ onset (FIG. 14B). At six months, these mice showed significantly reduced Aβ deposition in cortex, hippocampus (FIG. 14C), and thalamus. Methoxy-X04, a fluorescent Aβ-binding dye, revealed over two-fold higher signal in Ndst1^{CKO}; 5×FAD microglia vs. 5×FAD controls 3 hours after i.p. injection (FIG. 14D). Cultured Ndst1^{CKO}; 5×FAD microglia showed enhanced uptake of Aβ-FITC fibrils (FIG. 14E), aligning with a recent study linking reduced HS to increased Aβ uptake in BMDMs. These results demonstrate that lowering microglial HS enhances Aβ phagocytosis and reduces brain Aβ accumulation.

The present disclosure also shows that knockout of microglial Ndst1 improves cognition in 5×FAD mice. 5×FAD mice develop cognitive deficits by four months of age (FIG. 14B). To determine whether reducing microglial HS expression impacts cognition, behavioral tests are conducted on 6-7-month-old Ndst1^{CKO}; 5×FAD mice using the USF Behavior Core. In the two-day Novel Object Recognition (NOR) test, Ndst1^{CKO}; 5×FAD mice spent significantly more time exploring the novel object and showed a higher discrimination index than 5×FAD controls (FIG. 14F), indicating improved recognition memory. In the Y-Maze test, Ndst1^{CKO}; 5×FAD mice exhibited increased spontaneous alternations and fewer total arm entries (FIGS. 14G and 14H), reflecting enhanced working memory and reduced hyperactivity. Together, these results demonstrate that microglial Ndst1 deletion improves cognition in 5×FAD mice.

The data herein highlight a critical disease-modifying role for microglial HS in Aβ pathology. There was reduced microglial HS expression in AD patients, particularly in DAM, likely driven by distinct patterns of HS biosynthetic gene expression. In 5×FAD mice, microglia-specific deletion of Ndst1 lowered HS levels and significantly reduced Aβ accumulation. However, the structural changes in microglial HS, their broader impact on Aβ metabolism, tau pathology, neuroinflammation, neurodegeneration, and cognitive function, as well as the effects of Ndst1 deletion after Aβ onset, remain unclear. The present disclosure addresses these critical gaps and advances the understanding of microglial HS in AD pathogenesis.

Microglial Heparan Sulfate (HS) is expressed in human AD. To investigate microglial HS expression in human AD, forebrain sections are stained for microglia, Aβ plaques, and HS, and utilize the NanoString GeoMx Digital Spatial Profiler (DSP) platform at the Spatial Biology Core to spatially profile HS gene expression. Correlations among HS levels, HS gene expression, and DAM markers are examined in control and AD, comparing DAM and non-DAM. Gene Ontology enrichment analysis identify HS-modulated pathways, which are validated through fluorescence in situ hybridization (FISH) and protein staining. Twenty-one control and thirty-two AD brain specimens are obtained with detailed pathology information from Emory University's Goizueta AD Research Center. To enhance interpretability, preclinical AD specimens are also included, characterized by primary Aβ deposition, given the finding that Aβ directly downregulates the HS gene expression in microglia (FIG. 13G).

Microglial HS structures are analyzed in Ndst1^{CKO} mice. The anti-HS 10E4 antibody binds the NS-modified epitope of the HS chain. The reduced 10E4 staining of Ndst1^{CKO} microglia indicates that Ndst1 deletion reduces NS (FIG. 14A). The changes of Ndst1 deletion on NS, 2S, 6S, and 3S are quantitated in isolated microglia using disaccharide composition and specific 3S analyses.

The impact of Ndst1 deletion on amyloid precursor protein (APP) expression, Aβ production, distribution, degradation, and plaque properties is determined by the following: 1. Aβ production: APP undergoes amyloidogenic processing via β- and γ-secretases to generate Aβ peptides. HS inhibits β-secretase, reducing Aβ production. Since microglia express APP, it is assessed whether Ndst1 deficiency alters microglial Aβ production by immunoblotting APP fragments in cultured microglia; 2. Aβ distribution and degradation: Reduced total Aβ deposition is observed in Ndst1^{CKO}; 5×FAD mice (FIG. 14C) and is further stain for Aβ40 and Aβ42.

HS promotes Aβ aggregation and fibrillization. Total Aβ, Aβ40, Aβ42, and Aβ40/42 ratio is quantified in soluble, detergent-soluble, and insoluble brain fractions to assess the impact of Ndst1 deletion on these dynamic pools by ELISAs. Cellular degradation is one major pathway for Aβ clearance in the brain, with microglia acting as primary phagocytes and astrocytes contributing.

Ndst1 deletion enhances microglial Aβ phagocytosis (FIGS. 14D and 14E). Astrocyte phagocytosis is assessed using an in vivo Methoxy-X04-Aβ uptake assay, even though no changes in astrocyte number or morphology are observed by GFAP staining in Ndst1^{CKO}; 5×FAD mice; 3. Aβ plaque properties: Co-staining for Aβ and methoxy-X04 reveals distinct plaque types, with less compact and reduced filamentous Aβ plaques being more toxic than compact Aβ plaques. HS promotes Aβ fibrillization and co-deposits in plaques, and microglia influence Aβ plaque morphology. It is assessed if microglial Ndst1 deletion alters Aβ plaque compaction; 4. Aβ seeding: Microglia contribute to Aβ seeding, and the loss of TREM2 enhances this process. It was found that HS forms complexes with TREM2 on the microglial cell surface, thereby suppressing Aβ binding (FIGS. 18A, 18B, and 18C). It is also determined whether Ndst1 knockout reduces microglia-mediated Aβ seeding in 5×FAD mice. In brief, 5×FAD brain homogenates are injected into the hippocampus (CA1 region) of 3-week-old Ndst1^{CKO}; 5×FAD and 5×FAD mice, and then plaque formation is assessed after 4 weeks; and 5. Aβ propagation: Microglia facilitate the spread of Aβ into unaffected regions, depending on Cx3cr1 and Irf8. It is determined if this pathogenic process also depends on microglial HS. In brief, GFP-labeled cortical neurons are transplanted into Ndst1^{CKO}; 5×FAD and 5×FAD mice at young (9-10 weeks) and adult (8-11 months) ages, and assess microglial infiltration and Aβ deposition in grafts after 4 or 16 weeks.

The phosphorylation of Tau protein. Aβ accumulation leads to hyperphosphorylated tau in AD. This process is seen in 5×FAD mice at 4-6 months of age, including Ser396, Ser202/Thr205 (AT8), Thr212/Ser214 (AT100), and Thr231. Tissue immunostaining and phosphoproteomics of purified tau are assessed to investigate whether microglial Ndst1 deletion reduces tau phosphorylation in 5×FAD mice.

Although microglial and astrocyte numbers remain unchanged in Ndst1^{CKO}; 5×FAD mice, scRNA-seq shows upregulation of immune/inflammatory pathways (FIG. 15E). Pro-inflammatory cytokines, such as TNFα, IL-1β, and IL-6, are crucial in human AD and Aβ pathology in mouse models like 5×FAD. Pro-inflammatory (TNFα, IL-1β, IL-6) and anti-inflammatory (IL-10, IL-4, IL-13, TGF-β) cytokines are quantified using qRT-PCR, multiplex assays, and proteomics, and reanalyze scRNA-seq data to assess cytokine expression across cell types.

Fluoro-Jade C is used for degenerating neurons and NeuN and Cresyl Violet for neuronal morphology and loss, and synaptophysin and PSD-95 for synaptic loss. As Ndst1 deletion improves cognition in 6-7-month-old 5×FAD mice (FIGS. 14F, 14G, and 14H), it is further evaluated for learning and visuospatial memory using the Morris Water Maze in collaboration with the USF Behavioral Core.

The impact of microglial Ndst1 deficiency after Aβ deposition onset. A protective effect is observed in 6-month-old 5×FAD mice after inducing microglial Ndst1 deletion at one month of age, preceding Aβ deposition onset. To better understand this protective effect, microglial Ndst1 deletion are also induced at 6 months of age, after substantial Aβ deposition, and the outcomes at 11 months of age are assessed.

The analyses of human brain specimens reveal reduced HS expression in microglia from AD patients, with even lower levels in DAM compared to non-DAM populations, correlating with decreased expression of HS biosynthetic genes. In mouse models, Ndst1 deletion reduces microglial HS NS and downstream 2S, 6S, and 3S modifications, leading to decreased levels of total Aβ, Aβ40, and particularly Aβ42, which is more abundant in the parenchyma. This deletion promotes the formation of more compact, less toxic plaques and reduce Aβ seeding and propagation. While Ndst1 deletion may modestly increase microglial Aβ production due to loss of HS-mediated β-secretase inhibition, this effect is likely minimal given the low APP expression in microglia, even in AD. Additionally, Ndst1 deletion reduces tau phosphorylation, suppresses neuroinflammation, and improves cognitive performance in 5×FAD mice. Initiating Ndst1 deletion after Aβ deposition onset yields similar protective effects. Collectively, these outcomes establish a functional link between reduced microglial HS and neuroprotection in AD, highlighting microglial HS as a key modulator of Aβ clearance in AD and a therapeutic target.

Elucidation of the roles and underlying mechanisms of HS in regulating microglia functions in AD. Microglial Ndst1 deficiency promotes DAM generation and reprograms microglial functions in 5×FAD mice. To investigate the mechanisms underlying the protective effects of microglial Ndst1 deletion in 5×FAD mice, scRNA-seq was performed on microglia isolated from 6-month-old 5×FAD and Ndst1^{CKO}; 5×FAD male mice. Using the 10× Genomics platform, high-resolution transcriptomes was generated from 62,635 high-quality single cells, visualized via UMAP. Unsupervised clustering identified 13 brain cell populations, with microglia as the dominant cell type. Based on established microglial subpopulation signatures in 5×FAD mice, 11 microglial clusters were identified and HM became the focus, two transitional states (TM1 and TM2), and DAM, comprising 28,379 cells (FIGS. 15A and 15B). Notably, Ndst1^{CKO}; 5×FAD microglia showed a ˜10% increase in DAM compared to 5×FAD controls, along with shifts in TM1 and TM2 proportions, showing enhanced DAM generation (FIG. 15C). Transition efficiency analysis revealed that Ndst1 deletion reduced HM-to-TM1 transition but increased TM1-to-TM2 and TM2-to-DAM transitions, resulting in a net increase in TM1-to-DAM conversion (FIG. 15D). To refine these trajectories, RNA velocity analysis is applied using Velocyto. Gene set enrichment analysis of differentially expressed genes across HM, TM1, TM2, and DAM showed that Ndst deletion suppresses AD- and neurodegeneration-related pathways while upregulating immune response, phagosome, lysosome activity and MAPK signaling (FIG. 15E). These transcriptomic shifts align with enhanced microglial Aβ phagocytosis and clearance in Ndst1^{CKO}; 5×FAD mice. Together, these findings demonstrate that Ndst1 deletion promotes protective DAM generation and also broadly reprograms microglial functions to confer protection in AD.

Ndst1 deficiency upregulates microglial TREM2 expression. The scRNA-seq analysis of 6-month-old male Ndst1^{f/f} and Ndst1^{CKO} mice revealed that Ndst1 deletion upregulates TREM2 in microglia, specifically in DAM, as determined by subcluster analysis (FIGS. 16A and 16B). In the 5×FAD background, TREM2 expression was elevated across HM, TM1, TM2, and DAM. This upregulation was validated by qRT-PCR analyses of brain tissues (FIG. 16C) and immunofluorescence staining showing higher TREM2 expression on Ndst1^{CKO} microglia (FIG. 16D). As TREM2 signaling drives DAM development, these findings show that Ndst1 deletion upregulates TREM2 expression, promoting DAM generation and contributing to the protective phenotype observed in the Ndst1^{CKO}; 5×FAD mice.

HS binds TREM2, forming HS-TREM2 binary complexes on the human microglia cell surface. It has been reported that TREM2 ectodomains bind to the surfaces of THP-1 and Neuro2A cells in an HS-dependent manner. The present disclosure further showed that TREM2 interacts specifically with HS, but not chondroitin sulfate, on the endothelial cell surface. To investigate this interaction in microglia, human C20 microglial cells, which express both TREM2 and HS (FIG. 17A) were further examined. Confocal imaging revealed substantial co-localization of TREM2 with HS (Pearson's correlation coefficient=0.43), which was diminished after heparinase treatment (FIGS. 17A and 17B). Using proximity ligation assay (PLA), an abundance of HS-TREM2 binary complexes was detected on the C20 cell surface, which were markedly reduced in heparinase-treated and Ext1 knockdown (KD) cells, which lack HS expression (FIGS. 17C and 17D). These findings show that HS binds TREM2 in cis to form stable complexes on the microglial surface, showing a direct regulation of HS in modulating TREM2 signaling and microglial function.

Ndst1 deletion disrupts BMDM and microglia cell surface HS-TREM2 complex formation and heparin-binding blocks TREM2 from binding Aβ fibrils, showing Ndst1 deletion increases TREM2 binding of Aβ to enhance Aβ clearance. In PLA, primary mouse Ndst1^{KO} BMDMs from Tie2Cre⁺; Ndst1^{f/f} mice exhibit reduced HS-TREM2 complexes on the cell surface compared to Ndst1^{f/f} controls (FIG. 18A), with similar reductions observed in primary Ndst1^{CKO} microglia. These findings indicate that Ndst1 deletion impairs HS-TREM2 complex formation in Ndst1^{CKO}; 5×FAD microglia. Since TREM2 is a known receptor for Aβ fibrils in microglia, it was investigated whether HS binding interferes with TREM2-Aβ interaction. Using an ELISA-based assay with enoxaparin (a low molecular weight heparin) as an HS analog, enoxaparin prebinding was found to significantly inhibit TREM2 binding to Aβ fibrils (FIG. 18B). A complementary pull-down assay confirmed that TREM2 preincubated with enoxaparin or heparin failed to capture Aβ fibrils, whereas unbound TREM2 did so efficiently (FIG. 18C). These results show that HS-TREM2 complex formation suppresses TREM2-Aβ binding, thereby impairing Aβ phagocytosis (FIG. 12A). Ndst1 deletion reduces HS-TREM2 complex formation, freeing TREM2 to bind Aβ effectively and enhance its clearance (FIG. 12B). Given that Aβ-TREM2 signaling promotes DAM development, these findings also show that HS-TREM2 complex formation suppresses DAM generation in AD.

Herein critical insights into the role of Ndst1 in microglial biology and AD have been uncovered, but several key questions remain: 1. scRNASeq analysis show that Ndst1 deletion affects multiple microglial biological processes and functions, providing protective effects in 5×FAD mice, warranting further investigation; 2. The mechanism by which Ndst1 deletion upregulates TREM2 expression in microglia remains unknown; 3. The in vivo functional consequences of HS-TREM2 interaction in AD pathology remain unknown. The present disclosure addresses these critical gaps and more.

In Aβ pathology, microglia alter subcluster profiles, proliferate, migrate to cluster around, and phagocytose Aβ plaques. The present disclosure examines whether these alterations are affected by Ndst1 deletion.

Microglia subclusters. The scRNA-seq revealed no changes in the expression of Mki67 and Top2a, markers of proliferating microglia, or Cd74, a marker of MHCII microglia, indicating that Ndst1 deletion doesn't affect microglial proliferation or the MHCII microglia in 5×FAD mice. In contrast, upregulation of Ifitm3, Bst2, and Stat1 was observed showing an expansion of the Interferon-responsive (IFN-R) microglia. WeThe findings are validated at the mRNA and protein levels using qRT-PCR analysis of isolated microglia and tissue FISH and immunostaining.

Cytokine expression. The increase in the IFN-R microglial subpopulation shows that Ndst1 deletion may affect cytokine secretion. The scRNAseq data is analyzed to identify altered cytokine mRNAs in microglia in Ndst1^{CKO}; 5×FAD mice. Aβ induces microglia to secrete inflammatory cytokines IL-6 and MIP-1α. Ndst1-deficient microglia, with or without 5×FAD background, are treated with Aβ fibrils and the secreted cytokines are determined using a multiplex cytokine assay and proteomics analyses. These data are compared with global cytokine protein profiling data as previously described to determine the contribution of cytokines from Ndst1-deficient microglia to overall cytokine expression changes in Ndst1^{CKO}; 5×FAD mice.

Aβ induces microglial migration, clustering, proliferation, and survival. In AD, microglia migrate toward, cluster around, and clear Aβ plaques via phagocytosis. Using a transwell migration assay, Ndst1 KD enhanced human C20 microglia migration toward soluble oAβ (FIG. 19A). These findings are extended by testing primary microglia. TREM2 promotes microglial clustering around injected oAβ in the mouse brain. It is assessed whether Ndst1 deletion enhances microglial clustering around injected Aβ in Ndst1^{CKO} mice. Additionally, it is determined if Ndst1 KD protects microglia from oAβ-induced proliferation (Ki-67 staining) and apoptosis (TUNEL staining), in line with brain tissue analysis, even though the scRNA-seq did not observe microglial proliferation alteration in Ndst1^{CKO}; 5×FAD mice.

The phagocytosis and degradation of Aβ. Ndst1 deletion increases microglia Aβ phagocytosis (FIGS. 14D and 14E), thus Aβ degradation in cultured microglia is assessed. scRNA-seq showed that Ndst1 deletion upregulates phagosome function in microglia in 5×FAD mice (FIG. 15E) This is supported by higher CD68 mRNA and protein expression detected by qRT-PCR analysis of Ndst1^{CKO}; 5×FAD brain tissue and staining of oAβ-treated microglia, respectively, and enhanced endocytosis of the lysosomal tracker indicator Green DND-26 in Ndst1^{CKO}; 5×FAD microglia (FIG. 19B).

Heparan Sulfate regulates TREM2 expression. A TREM2 reporter assay is conducted using TREM2 Lenti-reporter Luc Vectors from Applied Biological Materials (Cat. No. 47700084) to confirm that Ndst1 deletion upregulates TREM2 expression in microglia. Additionally, multi-omics analyses of isolated primary microglia is performed, including ATAC-seq and H3K27ac ChIP-seq. ATAC-seq identifies differentially accessible regions of open chromatin, while H3K27ac ChIP-seq defines active gene regulatory elements, such as promoters and enhancers. These studies determine the microglia-specific core gene regulatory programs that drive the observed microglial cell state and TREM2 expression following Ndst1 deletion. To assess whether Ndst1 deletion upregulates TREM2 via a compensatory mechanism, Ndst1 is acutely deleted in Ndst1^{f/f} microglia using Adeno-Cre. TREM2 expression is measured at early (24-48 hours) and later (≥72 hours) time points, with and without oAβ treatment. An early increase in TREM2 expression shows direct regulation, while a delayed response shows compensation.

Heparan sulfate regulates TREM2 shedding. TREM2 is shed from the microglial surface by ADAM10 and ADAM17, producing soluble TREM2 (STREM2). STREM2 binds oAβ, blocks Aβ aggregation, and reduces Aβ neurotoxicity. It also supports microglial survival, cytokine release, and activation. In 5×FAD mice, sTREM2 enhances microglial proliferation, migration, plaque clustering, and Aβ clearance. HS inhibits ADAM12 activity, showing a broader regulation across the ADAM family. The present disclosure tests whether Ndst1 deletion increases sTREM2 levels in microglial culture media.

Heparan sulfate regulates Aβ-TREM2 interaction and signaling. The present disclosure also determines if Ndst1 deletion affects Aβ-TREM2 interaction on the microglial cell surface, downstream signaling, Aβ clearance, TREM2 expression and shedding.

The Aβ-TREM2 complex is present on microglial cell surfaces and in APP-transgenic TgCRND8 mice and human AD brains. To determine if Ndst1 deletion affects this interaction, Ndst1^{CKO} and Ndst1^{f/f} microglia are examined with or without 5×FAD: 1) Immunoprecipitation: Following incubating with Aβ fibrils, microglial lysates are precipitated with anti-TREM2 or anti-Aβ antibodies and analyzed by Western blot for both proteins; 2) PLA: As described in FIG. 17C, using anti-TREM2 and anti-Aβ antibodies to visualize complex formation.

Upon Aβ binding, TREM2 activates downstream signaling pathways including PI3K/AKT, Syk, mTOR and Rac1/Cdc42, which regulate microglia functions and TREM2 shedding (FIG. 12). These signaling responses are compared between Ndst1^{CKO} and Ndst1^{f/f} microglia and correlated with functional outcomes observed in the Microglia Functions section and TREM2 shedding and expression at mRNA and protein levels.

TREM2 KD abolished the Ndst1 KD-induced enhancement of Aβ-induced microglial migration (FIG. 19A) and Aβ uptake (FIG. 20C) in C20 microglia, showing that the Ndst1 KD effects are TREM2-dependent. To test this in vivo, conditional TREM2^{f/f} mice were obtained from the Jackson Laboratory (Strain #: 029853) and are bred to generate Ndst1^{CKO}; TREM2^{CKO}; 5×FAD mice by crossing with Ndst1^{CKO}; 5×FAD mice. The protective effects of microglial Ndst1 deletion are assessed in 5×FAD mice are lost in Ndst1^{CKO}; TREM2^{CKO}; 5×FAD mice by evaluating Aβ deposition, phagocytosis, cognition, and DAM development.

The systematic, unbiased scRNAseq with validation demonstrates that Ndst1 deletion significantly alters microglial subpopulations in 5×FAD mice, most notably increasing DAM and IFN-R clusters. These changes coincide with altered cytokine expression, particularly within the IFN-R population, contributing to a protective cytokine profile in the AD brain. Functional assays show that Ndst1 deletion enhances Aβ-induced microglial migration, clustering around plaques, and Aβ phagocytosis and degradation, correlating with increased lysosomal activity and TREM2 shedding. TREM2 reporter assay confirms upregulated TREM2 expression in Ndst1^{CKO} microglia. ATAC-seq and ChIP-seq assays identify the gene regulatory programs that upregulate TREM2 expression in microglia upon Ndst1 deletion, possibly minimally due to compensatory regulation. TREM2 pathway studies demonstrate that heparin prebinding inhibits the TREM2-Aβ interaction, while Ndst1 deletion disrupts HS-TREM2 complex formation, thereby enhancing Aβ-TREM2 binding and downstream activation of PI3K-Akt, Syk, mTOR, and Rac1/Cdc42 signaling upon Aβ stimulation, which correlates with enhanced migration, clustering, and phagocytosis. Finally, in vivo studies demonstrate that microglia-specific TREM2 deletion abrogates the protective effects of Ndst1 deletion in 5×FAD mice. These results elucidate that TREM2 pathway activation is the primary mechanism underlying the protective roles of microglial Ndst1 deletion in 5×FAD mice (FIGS. 12A and 12B).

Determining the TREM2-binding HS structure and targeting microglial HS for AD treatment. The binding of TREM2 to chemically modified heparins, including N-desulfated, 2-O-desulfated, and 6-O-desulfated heparins is examined using competitive SPR and a comprehensive HS microarray. This microarray enables the investigation of protein-HS interactions across various HS structures with defined chain lengths, sequences, and sulfation patterns. The findings revealed that TREM2 binding specifically requires 6S, but not NS or 2S, highlighting the critical role of 6S in HS-TREM2 interaction.

Knocking out Hs6st1 disrupts HS-TREM2 complex formation in microglia and BMDMs and increases microglial Aβ-induced migration and phagocytosis. Hs6st1-3 enzymes add 6S in HS biosynthesis (FIG. 13A). It was recently reported that knocking out Hs6st1 reduced TREM2 binding on mouse lung endothelial cells, indicating that HS requires 6S to bind TREM2 on the cell surface. Human microglia C20 cells express Hs6st1, and knocking down Hs6st1 disrupts HS-TREM2 complex formation, increasing Aβ uptake, similar to the effects of knocking down Ndst1 and Ext1 (FIGS. 20A, 20B, and 20C). Conversely, knocking down TREM2 drastically reduced Aβ uptake in C20 cells, confirming that TREM2 is the major receptor for Aβ uptake in microglia. Primary mouse microglia and BMDMs only express Hs6st1 (FIG. 13F). Tie2Cre⁺; Hs6st1^{f/f} mice were generated, where Hs6st1 is also knocked out (Hs6st1^{KO}) in BMDMs due to Tie2Cre expression in myeloid cells. PLA showed diminished HS-TREM2 complexes on the cell surface of Hs6st1^{KO} and heparinase-pretreated BMDMs (FIGS. 20D and 20E). PLA with primary Hs6st1^{KO} and Hs6st1^{f/f} microglia observed the same result. These data demonstrate that Hs6st1 is essential for HS to bind TREM2 in microglia and BMDMs, and its deletion enhances Aβ uptake, like Ndst1 deletion.

TREM2 knockdown abrogates Hs6st1-deletion-enhanced microglial responses to Aβ. TREM2 KD abolished the Hs6st1 KD-conferred enhancement of Aβ-induced microglial migration (FIG. 19A) and Aβ uptake (FIG. 20C), showing that the Hs6st1 KD effects are TREM2-dependent.

The analysis of human AD scRNA-seq data shows that although microglial Ndst1 expression is downregulated, it remains highly expressed and stable across early and late disease stages (FIG. 21). Immunostaining reveals a trend toward lower HS expression in DAM versus non-DAM microglia (FIGS. 13B and 13C), showing a dynamic regulatory role during disease progression. In 5×FAD mice, Ndst1 expression declines as HM transitions into DAM1 and DAM2 (FIG. 13F). Notably, microglia-specific Ndst1 deletion reduces Aβ pathology and improves cognition (FIG. 14). These findings indicate that microglial HS in AD, especially in non-DAM and transitional states, remains functionally relevant and therapeutically accessible.

Ndst1 deletion reduces NS, 2S, and 6S sulfation, broadly impacting HS-dependent pathways. In contrast, Hs6st1 deletion selectively reduces 6S, affecting only 6S-HS-dependent pathways. similar effects of Ndst1 and Hs6st1 deletion was observed on HS-TREM2 complex disruption, microglial Aβ uptake, and migration (FIGS. 18A, 19A, and 20C). It is essential to determine whether microglial Hs6st1 deletion confers protective effects, like Ndst1 deletion, in 5×FAD mice. Since Hs6st1 is highly and stably expressed in human microglia across all AD stages (FIG. 21), it represents a safer and more selective therapeutic target. Together, the protective effects of Ndst1 deletion and the benefits of Hs6st1 deletion support both genes as promising targets for AD treatment.

Determining the role of microglia Hs6st1 in Aβ pathology in 5×FAD mice. Tmem119-Cre^{ERT2} Hs6st1^{f/f}; 5×FAD mice were generated followed by TAM treatment to generate microglia-specific Hs6st1^{CKO}; 5×FAD mice. Hs6st1 deletion was induced at 1 and 6 months of age, and was examined for Aβ pathology, cognition, microglia functions, and TREM2 pathways at 6 and 11 months, respectively.

Replacement of microglia with Ndst1 or Hs6st1-deficient BMDMs alleviates Aβ pathology in 5×FAD mice. Microglia replacement with BMDMs is a promising strategy to modulate brain immune function and has shown therapeutic potential in neurodegenerative diseases. For example, replacing microglia in TREM2^{-/-}; 5×FAD mice with wildtype 5×FAD BMDMs resulted in >90% Iba1+ cells being BMDMs, enhancing microglial reactivity, reducing plaque load, and restoring DAM gene expression. In adult mice, including 5×FAD, microglia express only Ndst1 and Ndst2 from the Ndst family and Hs6st1 from the Hs6st family (FIG. 13F). Analysis of public RNA-seq data (NCBI BioProject: IPRJNA600501) shows that replacing BMDMs in the brain express 1.8-fold higher Ndst1 levels than microglia, with no change in other HS-related genes, including Hs6st1, showing a similar HS gene expression profile. This indicates that replacing microglia with Ndst1- and Hs6st1-deficient BMDMs can replicate the protective effects seen in Ndst1^{CKO}; 5×FAD and Hs6st1^{CKO}; 5×FAD mice. To test this, inducible macrophage-specific Cx3cr1CreER-EYFP knockin/knockout mice were obtained from the Jackson Laboratory and established protocols were followed that lead to near-complete microglial replacement with BMDMs. For Ndst1-deficient BMDM replacement, two donor lines: Cx3cr1CreER⁺Ndst1^{f/f} (Ndst1^{BMcf/f}) and Cx3cr1CreER⁺Ndst1^{f/f}; 5×FAD (Ndst1^{BMcf/f}; 5×FAD), were generated. To initiate replacement at Aβ deposition onset (FIGS. 22A and 22B), 5×FAD recipient mice received i.p. busulfan for 5 consecutive days starting at 3 weeks of age, followed by i.v. donor bone marrow (BM) transplantation at 4 weeks. At 6 weeks, mice are fed PLX5622 chow for 10 days to deplete resident microglia, then returned to standard chow. Complete microglial repopulation typically occurs within 1-2 weeks. Efficient microglia depletion by PLX5622 and late repopulation will be confirmed by Iba1 staining. At 9 weeks, mice received i.p. TAM for 4 days to induce Ndst1 deletion and Aβ pathology will be assessed at 27 weeks. This procedure ensures the observed effects are due to Ndst1-deficient BMDMs. To evaluate the impact after substantial Aβ accumulation, the same procedure begins at 6 months. Parallel experiments are tested in Hs6st1-deficient BMDM.

Microglia-specific deletion of Hs6st1 selectively reduces HS 6S and confers protective effects similar to those observed with Ndst1 deletion in 5×FAD mice. These effects include reduced Aβ accumulation, improved cognition, and increased DAM. TREM2 pathway analysis revealed that Hs6st1 deletion enhances microglial TREM2 expression, strengthens Aβ-TREM2 interactions, and promotes downstream signaling, leading to increased TREM2-dependent Aβ migration, clustering, and phagocytosis. In replacement studies, Ndst1BM and Hs6st1B1/BMDMs provide comparable protective effects in 5×FAD mice, mirroring those seen in Ndst1^{CKO}; 5×FAD and Hs6st1^{CKO}; 5×FAD models. Collectively, these findings provide new insights into the structural requirement of microglial HS in regulating Aβ clearance and TREM2 activation, establishing the therapeutic potential of targeting microglial HS in AD, with Hs6st1 emerging as a more selective target.

Mouse numbers are determined using power calculations based on preliminary data (power=0.85), with a conservative 1.5× adjustment. Correlation analyses use Pearson's or rank correlation coefficients. Group comparisons (two or more groups) are analyzed using one- or two-tailed Student's t-tests or ANOVA with appropriate post hoc tests (Tukey, Dunnett's, or Holm-Sidak). To ensure rigor, two or more antibodies per target were used where feasible. Data is collected from at least two, typically three, independent experiments and combined for statistical analysis. Core facility technicians are blinded to genotype and treatment. Data points are only excluded if a clear experimental issue is identified; in such cases, the entire experiment will be repeated. Given the higher incidence of AD in women, also reflected in APP transgenic models, we both sexes are included in all experiments and assess whether HS functions differ between male and female mice.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1
LWH Heparan Sulfate (HS) compound structures and TREM2 binding intensities.

| No. | Structure | Length | NS | 2S | 6S | 3S | Total-S | IdoA | Intensity |
|---|---|---|---|---|---|---|---|---|---|
| 73 | GlcNS-GlcA-GlcNS- | 12 | 6 | 4 | 0 | 0 | 10 | 4 | 389 |
| | IdoA2S-GlcNS- | | | | | | | | |
| | IdoA2S-GlcNS- | | | | | | | | |
| | IdoA2S-GlcNS- | | | | | | | | |
| | IdoA2S-GlcNS- | | | | | | | | |
| | GlcA-Az | | | | | | | | |
| 74 | GlcNS6S-GlcA- | 12 | 6 | 4 | 6 | 0 | 16 | 4 | 2060.75 |
| | GlcNS6S-IdoA2S- | | | | | | | | |
| | GlcNS6S-IdoA2S- | | | | | | | | |
| | GlcNS6S-IdoA2S- | | | | | | | | |
| | GlcNS6S-IdoA2S- | | | | | | | | |
| | GlcNS6S- | | | | | | | | |
| | GlcA-Az | | | | | | | | |
| 75 | GlcNS-GlcA-GlcNS- | 14 | 7 | 5 | 0 | 0 | 12 | 5 | 539.33 |
| | IdoA2S-GlcNS- | | | | | | | | |
| | IdoA2S-GlcNS- | | | | | | | | |
| | IdoA2S-GlcNS- | | | | | | | | |
| | IdoA2S-GlcNS- | | | | | | | | |
| | IdoA2S-GlcNS- | | | | | | | | |
| | GlcA-Az | | | | | | | | |
| 76 | GlcNS6S-GlcA- | 14 | 7 | 5 | 7 | 0 | 19 | 5 | 2201.83 |
| | GlcNS6S-IdoA2S- | | | | | | | | |
| | GlcNS6S-IdoA2S- | | | | | | | | |
| | GlcNS6S-IdoA2S- | | | | | | | | |
| | GlcNS6S-IdoA2S- | | | | | | | | |
| | GlcNS6S- | | | | | | | | |
| | IdoA2S-GlcNS6S- | | | | | | | | |
| | GlcA-Az | | | | | | | | |
| 77 | GlcNS-GlcA- | 18 | 9 | 7 | 0 | 0 | 16 | 7 | 742.83 |
| | GlcNS6S-IdoA2S- | | | | | | | | |
| | GlcNS6S-IdoA2S- | | | | | | | | |
| | GlcNS6S-IdoA2S- | | | | | | | | |
| | GlcNS6S-IdoA2S- | | | | | | | | |
| | GlcNS6S- | | | | | | | | |
| | IdoA2S-GlcNS6S- | | | | | | | | |
| | IdoA2S-GlcNS6S- | | | | | | | | |
| | IdoA2S-GlcNS- | | | | | | | | |
| | GlcA-Az | | | | | | | | |
| 78 | GlcNS6S-GlcA- | 18 | 9 | 7 | 9 | 0 | 25 | 7 | 3384.58 |
| | GlcNS6S-IdoA2S- | | | | | | | | |
| | GlcNS6S-IdoA2S- | | | | | | | | |
| | GlcNS6S-IdoA2S- | | | | | | | | |
| | GlcNS6S-IdoA2S- | | | | | | | | |
| | GlcNS6S- IdoA2S- | | | | | | | | |
| | GlcNS6S-IdoA2S- | | | | | | | | |
| | GlcNS6S-IdoA2S- | | | | | | | | |
| | GlcNS6S-GlcA-Az | | | | | | | | |
| 84 | GlcNS6S-GlcA- | 12 | 6 | 4 | 6 | 1 | 17 | 4 | 2756.75 |
| | GlcNS6S3S-IdoA2S- | | | | | | | | |
| | GlcNS6S-IdoA2S- | | | | | | | | |
| | GlcNS6S-IdoA2S- | | | | | | | | |
| | GlcNS6S-IdoA2S- | | | | | | | | |
| | GlcNS6S-GlcA-Az | | | | | | | | |
| 96 | GlcA-GlcNS-GlcA- | 9 | 4 | 0 | 0 | 0 | 4 | 1 | 1452.83 |
| | GlcNS-GlcA-GlcNS- | | | | | | | | |
| | IdoA-GlcNS-GlcA- | | | | | | | | |
| | Az | | | | | | | | |
| 68 | GlcA-GlcNS-GlcA- | 9 | 4 | 1 | 0 | 0 | 4 | 1 | 273.67 |
| | GlcNS-GlcA-GlcNS- | | | | | | | | |
| | IdoA2S-GlcNS- | | | | | | | | |
| | GlcA-Az | | | | | | | | |
| 23 | GlcA-GlcNS-GlcA- | 9 | 4 | 0 | 0 | 0 | 4 | 0 | 104.17 |
| | GlcNS-GlcA- | | | | | | | | |
| | GlcNS-GlcA- | | | | | | | | |
| | GlcNS-GlcA-Az | | | | | | | | |
| 24 | GlcNS-GlcA- | 12 | 6 | 0 | 0 | 0 | 6 | 0 | 247.42 |
| | GlcNS-GlcA- | | | | | | | | |
| | GlcNS-GlcA- | | | | | | | | |
| | GlcNS-GlcA- | | | | | | | | |
| | GlcNS-GlcA- | | | | | | | | |
| | GlcNS-GlcA-Az | | | | | | | | |
| 47 | GlcNS6S-GlcA- | 12 | 6 | 0 | 6 | 0 | 12 | 0 | 111.75 |
| | GlcNS6S-GlcA- | | | | | | | | |
| | GlcNS6S-GlcA- | | | | | | | | |
| | GlcNS6S-GlcA- | | | | | | | | |
| | GlcNS6S-GlcA- | | | | | | | | |
| | GlcNS6S-GlcA-Az | | | | | | | | |
| 48 | GlcNS6S-GlcA- | 18 | 9 | 0 | 9 | 0 | 18 | 0 | 389.08 |
| | GlcNS6S-GlcA- | | | | | | | | |
| | GlcNS6S-GlcA- | | | | | | | | |
| | GlcNS6S-GlcA- | | | | | | | | |
| | GlcNS6S-GlcA- | | | | | | | | |
| | GlcNS6S-GlcA- | | | | | | | | |
| | GlcNS6S-GlcA- | | | | | | | | |
| | GlcNS6S-GlcA- | | | | | | | | |
| | GlcNS6S-GlcA- | | | | | | | | |
| | Az | | | | | | | | |

TABLE 2
Protein-ligand interactions between charged moieties
in the TREM2-heparin complex. Hydrogen bonds are
shown as ( - - - ) and salt bridges as ( . . . ).

| Octa-saccharide | |
|---|---|
| residue | Interactions |
| A | O₂—SO₃ - - - R76, O₂—SO₃ - - - R77 |
| B | O₆—SO₃ - - - R76 |
| C | COO - - - S65, COO ... R77 |
| D | O₆—SO₃ - - - R62 |
| E | COO - - - S65, COO ... R₄₇, O₂-SO₃ - - - R62 |
| F | N—SO₃ - - - R47, N-SO₃ - - - N68 |
| G | O₂—SO₃ - - - N68 |
| H | N—SO₃ - - - W7O |

^{a}Values in degrees.
^{b}Φ: O5-C1-O(x)-C(x), ψ: C1-O(x)-C(x)-C(x-1).

TABLE 3
Glycosidic ϕ/ψ-torsion angles^{a, b} before and after docking.

| Octa-saccharide | | |
|---|---|---|
| Linkage | Before | After |
| A-B | −51.3 / 121.4 | −85.0 / 111.2 |
| B-C | 67.8 / 110.8 | 96.9 / 88.9 |
| C-D | −51.3 / 121.4 | −106.4 / 53.9 |
| D-E | 60.4 / 104.1 | 97.4 / 15.1 |
| E-F | −49.1 / 127.1 | −98.9 / 102.0 |
| F-G | 62.4 / 107.2 | 109.1 / 79.9 |
| G-H | −50.6 / 123.3 | 139.7 / 107.5 |

SEQUENCES
1.
Human Trem2 Forward Primer
SEQ ID NO: 1
ATGATGCGGGTCTCTACCAGTG
2.
Human Trem2 Reverse Primer
SEQ ID NO: 2
GCATCCTCGAAGCTCTCAGACT
3.
Human Ext1 Forward Primer
SEQ ID NO: 3
GCTCTTGTCTCGCCCTTTTGT
4.
Human Ext1 Reverse Primer
SEQ ID NO: 4
GTGGTGCAAGCCATTCCTAC
5.
Human Ext2 Forward Primer
SEQ ID NO: 5
ATGTGTGCGTCGGTCAAGTAT
6.
Human Ext2 Reverse Primer
SEQ ID NO: 6
AGAATGGGGCCAAAACTGAAA
7.
Human Hst6st1 Forward Primer
SEQ ID NO: 7
ACGCCCAGGAAGTTCTACTAC
8.
Human Hst6st1 Reverse Primer
SEQ ID NO: 8
GTTGTACGGGCAGTCCATGAA
9.
Human Hst6st2 Reverse Primer
SEQ ID NO: 9
CATGGGGCCGAAAATCTTGGA
10.
Human Hst6st3 Forward Primer
SEQ ID NO: 10
TCCAGTGTCACGTTACCTGAG
11.
Human Hst6st3 Reverse Primer
SEQ ID NO: 11
TGTAGGTGCAATCCATAAACTCC
12.
Human Gapdh Forward Primer
SEQ ID NO: 12
GTCTCCTCTGACTTCAACAGCG
13.
Human Gapdh Reverse Primer
SEQ ID NO: 13
ACCACCCTGTTGCTGTAGCCAA
14.
Human Ext1 DsiRNA
SEQ ID NO: 14
GUCCUGAGUCUGGAUACUUUAGACA
15.
Human Hst6st1 DsiRNA
SEQ ID NO: 15
CUACGAGAAGAAGUACUACUUCCCG
16.
Human Trem2 DsiRNA
SEQ ID NO: 16
AACACCUGACAACUUCUGAAUAUUG
17.
HS6ST Inhibitor
SEQ ID NO: 17
RGWRGEKIGN
18.
HS6ST inhibitor
SEQ ID NO: 18
NMQALSMPVT

What is claimed is:

1. A method of treating a neurodegenerative disease, the method comprising administering to a subject in need thereof a pharmaceutical composition comprising at least one inhibitor of a heparan sulfate (HS) biosynthetic enzyme, wherein the at least one inhibitor decreases formation of a HS-Triggering Receptor Expressed on Myeloid Cells-2 (HS-TREM2) complex and decreases accumulation of Aβ relative to an untreated control.

2. The method of claim 1, wherein the at least one inhibitor of a HS biosynthetic enzyme comprises a peptide, an oligonucleotide, a small molecule, or an immunoglobulin.

3. The method of claim 1, wherein the at least one inhibitor of a HS biosynthetic enzyme comprises an inhibitor of N-deacetylase/N-sulfotransferases-1 (NDST1), an inhibitor of a Exostosin (EXT) glycosyltransferase, an inhibitor of a HS 6-O-sulfotransferase (HS6ST), an inhibitor of a glucuronyl C5-epimerase (Glce), or a combination thereof.

4. The method of claim 3, wherein the inhibitor of NDST1 inhibits binding between HS and TREM2.

5. The method of claim 3, wherein the inhibitor of the EXT glycosyltransferase inhibits adding a sugar molecule to a heparan sulfate chain.

6. The method of claim 3, wherein the EXT glycosyltransferase comprises an EXT1 or an EXT2.

7. The method of claim 3, wherein the HS6ST comprises HS6ST1, HS6ST2, HS6ST3, or any variant thereof.

8. The method of claim 3, wherein the inhibitor of HS6ST or the inhibitor of Glce inhibits a modification to HS.

9. The method of claim 8, wherein the modification comprises sulfonation to a glucosamine residue of HS.

10. The method of claim 8, wherein the modification comprises converting glucuronic acid (GlcA) to iduronic acid (IdoA).

11. The method of claim 1, wherein the neurodegenerative disease comprises Alzheimer's Disease (AD).

12. The method of claim 1, wherein the method promotes phagocytosis and degradation of Aβ.

13. A method of treating a neurodegenerative disease, the method comprising administering to a subject in need thereof a pharmaceutical composition comprising at least one agonist of a Triggering Receptor Expressed on Myeloid Cells-2 (TREM2), wherein the at least one agonist of TREM2 increases binding of amyloid-β (Aβ) to the TREM2 and decreases accumulation of Aβ relative to an untreated control.

14. The method of claim 13, wherein the at least one agonist of TREM2 comprises a peptide, an oligonucleotide, a small molecule, or an immunoglobulin.

15. The method of claim 13, wherein the neurodegenerative disease comprises Alzheimer's Disease (AD).

16. The method of claim 13, wherein the method promotes phagocytosis and degradation of Aβ.

17. A method of reducing accumulation of Aβ, comprising: contacting the cell with at least one inhibitor of a heparan sulfate (HS) biosynthetic enzyme, at least one agonist of a Triggering Receptor Expressed on Myeloid Cells-2 (TREM2), or a combination thereof, wherein the method decreases formation of a HS-TREM2 complex and increases binding of Aβ to the TREM2.

18. The method of claim 17, wherein the at least one inhibitor of a HS biosynthetic enzyme comprises a peptide, an oligonucleotide, a small molecule, or an immunoglobulin.

19. The method of claim 17, wherein the at least one agonist of TREM2 comprises a peptide, an oligonucleotide, a small molecule, or an immunoglobulin.

20. The method of claim 17, wherein the method treats or prevents Alzheimer's disease.


SEQUENCE LISTING (18 sequences)

SEQ ID NO: 1 - DNA, 22 bp, synthetic construct
  misc_feature 1..22: note = Description of sequence: Primer, name: Human Trem2 Forward Primer
  source 1..22: mol_type = other DNA
atgatgcgggtctctaccagtg

SEQ ID NO: 2 - DNA, 22 bp, synthetic construct
  misc_feature 1..22: note = Description of sequence: Primer, name: Human Trem2 Reverse Primer
  source 1..22: mol_type = other DNA
gcatcctcgaagctctcagact

SEQ ID NO: 3 - DNA, 21 bp, synthetic construct
  misc_feature 1..21: note = Description of sequence: Primer, name: Human Ext1 Forward Primer
  source 1..21: mol_type = other DNA
gctcttgtctcgcccttttgt

SEQ ID NO: 4 - DNA, 20 bp, synthetic construct
  misc_feature 1..20: note = Description of sequence: Primer, name: Human Ext1 Reverse Primer
  source 1..20: mol_type = other DNA
gtggtgcaagccattcctac

SEQ ID NO: 5 - DNA, 21 bp, synthetic construct
  misc_feature 1..21: note = Description of sequence: Primer, name: Human Ext2 Forward Primer
  source 1..21: mol_type = other DNA
atgtgtgcgtcggtcaagtat

SEQ ID NO: 6 - DNA, 21 bp, synthetic construct
  misc_feature 1..21: note = Description of sequence: Primer, name: Human Ext2 Reverse Primer
  source 1..21: mol_type = other DNA
agaatggggccaaaactgaaa

SEQ ID NO: 7 - DNA, 21 bp, synthetic construct
  misc_feature 1..21: note = Description of sequence: Primer, name: Human Hst6st1 Forward Primer
  source 1..21: mol_type = other DNA
acgcccaggaagttctactac

SEQ ID NO: 8 - DNA, 21 bp, synthetic construct
  misc_feature 1..21: note = Description of sequence: Primer, name: Human Hst6st1 Reverse Primer
  source 1..21: mol_type = other DNA
gttgtacgggcagtccatgaa

SEQ ID NO: 9 - DNA, 21 bp, synthetic construct
  misc_feature 1..21: note = Description of sequence: Primer, name: Human Hst6st2 Reverse Primer
  source 1..21: mol_type = other DNA
catggggccgaaaatcttgga

SEQ ID NO: 10 - DNA, 21 bp, synthetic construct
  misc_feature 1..21: note = Description of sequence: Primer, name: Human Hst6st3 Forward Primer
  source 1..21: mol_type = other DNA
tccagtgtcacgttacctgag

SEQ ID NO: 11 - DNA, 23 bp, synthetic construct
  misc_feature 1..23: note = Description of sequence: Primer, name: Human Hst6st3 Reverse Primer
  source 1..23: mol_type = other DNA
tgtaggtgcaatccataaactcc

SEQ ID NO: 12 - DNA, 22 bp, synthetic construct
  misc_feature 1..22: note = Description of sequence: Primer, name: Human Gapdh Forward Primer
  source 1..22: mol_type = other DNA
gtctcctctgacttcaacagcg

SEQ ID NO: 13 - DNA, 22 bp, synthetic construct
  misc_feature 1..22: note = Description of sequence: Primer, name: Human Gapdh Reverse Primer
  source 1..22: mol_type = other DNA
accaccctgttgctgtagccaa

SEQ ID NO: 14 - RNA, 25 nt, synthetic construct
  misc_feature 1..25: note = Description of sequence: siRNA name: Human Ext1 DsiRNA
  source 1..25: mol_type = other RNA
gtcctgagtctggatactttagaca

SEQ ID NO: 15 - RNA, 25 nt, synthetic construct
  misc_feature 1..25: note = Description of sequence: siRNA name: Human Hst6st1 DsiRNA
  source 1..25: mol_type = other RNA
ctacgagaagaagtactacttcccg

SEQ ID NO: 16 - RNA, 25 nt, synthetic construct
  misc_feature 1..25: note = Description of sequence: siRNA name: Human Trem2 DsiRNA
  source 1..25: mol_type = other RNA
aacacctgacaacttctgaatattg

SEQ ID NO: 17 - protein, 10 aa, synthetic construct
  REGION 1..10: note = Description of sequence: Peptide, name: HS6ST Inhibitor
RGWRGEKIGN

SEQ ID NO: 18 - protein, 10 aa, synthetic construct
  REGION 1..10: note = Description of sequence: Peptide, name: HS6ST Inhibitor
NMQALSMPVT